(19) United States
(12) Patent Application Publication
(10) Pub. No.: US 2025/0383333 A1
(43) Pub. Date: Dec. 18, 2025

(54) DETERMINATION OF PARTICULATE CONTAMINANTS IN PARTICLES AND COMPOSITIONS

(71) Applicant: Elektrofi, Inc., Boston, MA (US)

(72) Inventors: Aditya Krishna, Boston, MA (US); Greg Fahs, Waltham, MA (US); Nicholas Larson, Somerville, MA (US); Gabriel Lind, Brookline, MA (US)

(21) Appl. No.: 19/236,596

(22) Filed: Jun. 12, 2025

Related U.S. Application Data
(60) Provisional application No. 63/659,064, filed on Jun. 12, 2024.
(60) Provisional application No. 63/710,117, filed on Oct. 22, 2024.

(51) Int. Cl. G01N 33/15; C12Q 1/37

(52) U.S. Cl. CPC G01N 33/15; C12Q 1/37

(57) ABSTRACT

Provided herein are methods of detecting particulate contaminants in particles or a composition comprising particles, wherein the particles comprise a therapeutic or diagnostic agent, and wherein the particulate contaminants do not comprise the therapeutic or diagnostic agent.

### RELATED APPLICATIONS

This application claims the benefit of U.S. Provisional Application No. 63/710,117, filed on Oct. 22, 2024, and U.S. Provisional Application No. 63/659,064, filed on Jun. 12, 2024. The entire teachings of the above applications are incorporated herein by reference.

### BACKGROUND

There is a growing interest in developing methods to maintain the integrity of products, such as pharmaceutical products, in order to protect public health. Such methods may be employed to detect contaminants such as heavy metals, microbial pathogens, and residual solvents.

### SUMMARY

Disclosed herein are methods of detecting particulate contaminants in particles or compositions comprising particles, wherein the particles comprise a therapeutic or diagnostic agent, and wherein the particulate contaminants do not comprise the therapeutic or diagnostic agent. The method generally comprises: a) mixing the particles or the composition comprising particles with a dissolution media under conditions in which the particles dissolve to produce a mixture (e.g., a solution) comprising soluble therapeutic or diagnostic agent; b) adding at least one destructive reagent (e.g., denaturing agent) to the mixture comprising the soluble therapeutic or diagnostic agent to degrade the therapeutic or diagnostic agent; c) filtering the mixture through a filtration apparatus comprising a first membrane filter; and d) inspecting the surface of the first membrane filter for the presence of particulate contaminants.

### BRIEF DESCRIPTION OF THE DRAWINGS

The foregoing will be apparent from the following more particular description of example embodiments, including those illustrated in the drawings interspersed herein. The drawings are not necessarily to scale, emphasis instead being placed upon illustrating embodiments.

FIG. 1: Filtration Apparatus Assembly.

FIG. 2: Representative image of segmentation that is not of a large enough range (missing particle in the top left).

FIG. 3: Representative image of segmentation that is of too large a range (many artifactual segmentation objects).

FIG. 4: Representative image of segmentation that is of a proper range.

FIG. 5: Representative image of an incomplete segmentation of a fiber with an inhomogeneous contrast distribution.

FIG. 6: Image re-segmented using the “combine regions” function in Zen Core.

FIG. 7: Number of particles of various sizes per vial, based on the methods outlined in Table 3.

FIG. 8: Number of subvisible particles per mg of various sizes (i.e., >2 μm, >10 μm, >25 μm), based on the methods outlined in Table 3.

FIG. 9: Detection rates of >250 μm particles.

### DETAILED DESCRIPTION

A description of example embodiments follows.

The present disclosure relates to methods of detecting particulate contaminants in agent-containing particles or a composition comprising agent-containing particles (e.g., particles comprising a therapeutic or diagnostic agent such as a therapeutic biologic or salt thereof, or protein particles), wherein the particulate contaminants do not comprise the agent (e.g., a therapeutic or diagnostic agent), and wherein the method comprises one or more of: a) mixing the particles or the composition comprising particles with a dissolution media under conditions in which the particles dissolve to produce a mixture (e.g., a solution) comprising soluble agent (e.g., soluble therapeutic or diagnostic agent, soluble proteins); b) adding at least one destructive reagent (e.g., denaturing agent) to the mixture comprising the soluble therapeutic or diagnostic agent to degrade the therapeutic or diagnostic agent; c) filtering the mixture through a filtration apparatus (e.g., a filter flask, a depth filter, a membrane filter, a sieve) comprising a first membrane filter; and d) inspecting the surface of the first membrane filter for the presence of particulate contaminants. In some embodiments, the agent is a therapeutic or diagnostic agent (e.g., a protein, such as an antibody or a fragment thereof). In some embodiments, the composition comprises particles suspended in a liquid.

In some embodiments, methods of the present disclosure are used to quantify and measure the sizes of any particulate contaminants (e.g., contaminants in a drug product). For example, methods of the present disclosure are useful for analyzing a drug product comprising protein particles (e.g., a suspension of microparticles in a carrier liquid), in which the presence of both intrinsic and extrinsic contaminants may be detected by firstly dissolving the protein microparticles and any inherent proteinaceous insoluble aggregates by using an aqueous destructive treatment buffer.

In some embodiments, the particulate contaminants are non-protein particulate contaminants. Non-limiting examples of non-protein particulate contaminants include a metal, silica (e.g., glass), titania, a metal salt, a metal oxide, a metal nitride, a metal sulfide, a metal alkoxide, a polymer (e.g., rubber), or a combination thereof.

In some embodiments, the particular contaminants (e.g., non-protein particulate contaminants) are greater than about 1 μm in average diameter (e.g., greater than about 5 μm in average diameter, greater than about 6 μm in average diameter, greater than about 7 μm in average diameter, greater than about 8 μm in average diameter, greater than about 10 μm in average diameter, greater than about 15 μm in average diameter, greater than about 20 μm in average diameter, greater than about 25 μm in average diameter, greater than 50 μm in average diameter, greater than about 100 μm in average diameter, greater than about 150 μm in average diameter, greater than about 200 μm in average diameter, greater than about 250 μm in average diameter, etc.).

In some embodiments, an agent in particles (e.g., microparticles) is released into a dissolution media with monitoring of agent concentration. The agent concentration may be monitored at discrete time points or continuously monitored. For example, agent (e.g., protein) concentration may be monitored by UV-Vis spectroscopy, Kjeldahl method, Biuret method, Lowry assay, Bicinchoninic acid (BCA) assay, Bradford assay, or enzyme-linked immunosorbent assay (ELISA), and may be combined with separation methods (e.g., hydrophobic interaction column chromatography, size exclusion chromatography, ion exchange column chromatography, affinity chromatography, centrifugation, gel electrophoresis, molecular sieve chromatography, etc.). For example, a protein species is separated by size-exclusion chromatography, detected by UV-Vis spectroscopy at 280 nm, and the amount of protein is determined based on total peak response and is reported on a relative percentage basis.

In some embodiments, a dissolution media comprises a surfactant. In some embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, sodium laureth sulfate, lecithin, or a combination thereof. In some embodiments, the surfactant includes, but is not limited to: (i) cationic surfactants such as; cetyltrimethylammonium chloride, hexadecyltrimethylammonium chloride, benzalkonium chloride, benzethonium chloride, dioctadecyldimethylammonium bromide; (ii) anionic surfactants such as magnesium stearate, sodium dodecyl sulfate, dioctyl sodium sulfosuccinate, sodium myreth sulfate, perfluorooctanesulfonate, alkyl ether phosphates; (iii) non-ionic surfactants such as alkylphenol ethoxylates (TritonX-100), fatty alcohol ethoxylates (octaethylene glycol monododecyl ether, cocamide diethanolamine, poloxamers, glycerolmonostearate, fatty acid esters of sorbitol (sorbitan monolaurate, Tween 80, Tween 20; and (iv) zwitterionic surfactants such as cocamidopropyl hydroxysultaine, and 3-[(3-Cholamidopropyl)dimethylammonio]-1-propanesulfonate (CHAPS). In other embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, or a combination thereof. In certain embodiments, the surfactant is polysorbate, docusate or lecithin. In some embodiments, the surfactant is polysorbate 20, polysorbate 60, or polysorbate 80. In some embodiments, the surfactant is polysorbate 20. In some embodiments, the dissolution media is 0.5% v/v polysorbate 20 in 1× phosphate buffered saline (PBS) at a pH of about 7.4.

In some embodiments, mixing particles or a composition comprising particles with a dissolution media (e.g., protein dissolution media) comprises diluting the particles or the composition comprising particles using the dissolution media to produce a mixture. In some embodiments, the particles or the composition comprising particles is diluted to an agent (e.g., protein) concentration of from about 1 mg/ml to about 100 mg/ml (e.g., about 1 mg/ml to about 50 mg/ml, about 1 mg/ml to about 40 mg/ml, about 1 mg/ml to about 30 mg/ml, about 10 mg/ml to about 30 mg/ml, etc.) using the dissolution media. In some embodiments, the particles or the composition comprising particles is diluted to an agent concentration of about 20 mg/ml using the dissolution media.

In some embodiments, a dissolution media is mixed with particles or a composition comprising particles using a mixer (e.g., rotators, shakers, rockers, combinations thereof, etc.). Non-limiting example of mixers include nutating mixers and orbital mixers. In some embodiments, a dissolution media is mixed with the particles or the composition using a mixer at a speed of from about 10 revolutions per minute (rpm) to about 200 rpm (e.g., about 10 rpm to about 150 rpm, about 30 rpm to about 150 rpm, about 50 rpm to about 150 rpm, about 60 rpm to about 150 rpm, about 70 rpm to about 150 rpm, about 100 rpm to about 150 rpm, etc.). In some embodiments, the dissolution media is mixed with the particles or the composition using a mixer at about 60 rpm. In other embodiments, the dissolution media is mixed with the particles or the composition using a mixer at about 120 rpm.

In some embodiments, a dissolution media is mixed with particles or a composition comprising particles for a time period of from about 1 minute to about 5 hours (e.g., about 1 minute to about 4 hours, about 1 minute to about 3 hours, about 1 minute to about 2 hours, about 30 minutes to about 100 minutes, etc.). In some embodiments, the dissolution media is mixed with the particles or the composition comprising particles for about 90 minutes.

In some embodiments, adding at least one destructive reagent (e.g., protein destructive reagent) to a mixture comprising soluble agent comprises diluting the mixture comprising soluble agent (e.g., therapeutic or diagnostic agent, proteins) using a first destructive reagent. In some embodiments, a mixture comprising soluble therapeutic or diagnostic agent is diluted to a therapeutic or diagnostic agent concentration of from about 1 mg/ml to about 100 mg/ml (e.g., about 1 mg/ml to about 50 mg/ml, about 5 mg/ml to about 30 mg/ml, about 1 mg/ml to about 40 mg/ml, about 1 mg/ml to about 30 mg/ml, about 1 mg/ml to about 20 mg/ml, etc.) using a first destructive reagent. In some embodiments, the mixture comprising the soluble therapeutic or diagnostic agent is diluted to a therapeutic or diagnostic agent concentration of about 10 mg/ml using the first destructive reagent. In some embodiments, a solution comprising non-protein particulate contaminants and soluble proteins is diluted to a protein concentration of about 10 mg/ml using a first destructive reagent.

Non-limiting examples of destructive reagents include reagents (e.g., solutions) comprising denaturants such as reducing agents, oxidizing agents, and surfactants (e.g., polysorbate, magnesium stearate, guanidine hydrochloride, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, sodium laureth sulfate, lecithin, etc.), chaotropic agents (e.g., urea, etc.), digestion agents (e.g., enzymes, trypsin, lys-c, etc.), acids (e.g., hydrochloric acid, etc.), and combinations thereof. A destructive reagent may be prepared by filtering a solution comprising a denaturant. For example, a solution comprising a denaturant may be filtered through a membrane filter or sieve. In some embodiments, the first destructive reagent comprises sodium dodecyl sulfate. In some embodiments, the first destructive reagent is a 10% sodium dodecyl sulfate (SDS) solution.

In some embodiments, adding at least one destructive reagent to a mixture comprising soluble agent (e.g., soluble therapeutic or diagnostic agent) further comprises mixing the mixture and a first destructive reagent. In some embodiments, a mixture comprising soluble therapeutic or diagnostic agent is mixed with a first destructive reagent for a time period of from about 1 minute to about 5 hours (e.g., about 1 minute to about 4 hours, about 1 minute to about 3 hours, about 1 minute to about 2 hours, about 30 minutes to about 100 minutes, etc.). In some embodiments, the mixture comprising the soluble therapeutic or diagnostic agent is mixed with the first destructive reagent for at least about 90 minutes. In some embodiments, the mixture comprising the soluble therapeutic or diagnostic agent is mixed with the first destructive reagent for about 90 minutes. In some embodiments, the first destructive reagent comprises sodium dodecyl sulfate, urea, guanidine hydrochloride, or a combination thereof. In some embodiments, the first destructive reagent comprises urea.

In some embodiments, the concentration of the first destructive reagent is from about 0.1 M to about 50 M (e.g., about 0.1 M to about 40 M, about 0.1 M to about 30 M, about 0.1 M to about 20 M, about 0.1 M to about 15 M, about 0.1 M to about 10 M, about 0.5 M to about 10 M, about 1 M to about 10 M, about 1.5 M to about 10 M, about 2 M to about 10 M, etc.). In some embodiments, the concentration of the first destructive reagent is from about 2 M to about 8 M.

In some embodiments, adding at least one destructive reagent to a mixture comprising soluble agent (e.g., soluble therapeutic or diagnostic agent) further comprises heating the mixture. In some embodiments, the mixture is heated at from about 40° C. to about 100° C. (e.g., about 40° C. to about 100° C., about 40° C. to about 90° C., about 50° C. to about 90° C., about 60° C. to about 90° C., about 60° C. to about 80° C., etc.). In some embodiments, the temperature of the mixture is heated at about 70° C. In some embodiments, the mixture is heated for a time period of about 1 minute to about 3 hours (e.g., about 1 minute to about 3 hours, about 1 minute to about 3 hours, about 1 minute to about 2 hours, about 1 minute to about 1 hour, about 10 minutes to about 1 hour, etc.). In some embodiments, the mixture is heated for about 30 minutes. In some embodiments, the mixture is heated at about 70° C. for about 30 minutes.

In some embodiments, adding at least one destructive reagent to a mixture comprising soluble agent (e.g., soluble therapeutic or diagnostic agent) further comprises heating the mixture and cooling the mixture. In some embodiments, the mixture is cooled to from about 20° C. to about 25° C. In some embodiments, the adding further comprises heating the mixture at from about 40° C. to about 100° C. after diluting the mixture (e.g., diluting the mixture comprising the soluble therapeutic or diagnostic agent to a therapeutic or diagnostic agent concentration of from about 1 mg/ml to about 50 mg/ml using a first destructive reagent) and cooling the mixture to from about 20° C. to about 25° C.

In some embodiments, a mixture comprising soluble therapeutic or diagnostic agent is mixed with a first destructive reagent using a mixer (e.g., rotators, shakers, rockers, combinations thereof, etc.). Non-limiting example of mixers include nutating mixers and orbital mixers. In some embodiments, a solution comprising particulate contaminants and soluble agent is mixed with a first destructive reagent at a speed of from about 10 revolutions per minute (rpm) to about 200 rpm (e.g., about 10 rpm to about 150 rpm, about 30 rpm to about 150 rpm, about 10 rpm to about 100 rpm, about 10 rpm to about 90 rpm, about 20 rpm to about 80 rpm, about 50 rpm to about 70 rpm, etc.). In some embodiments, the mixture comprising the soluble therapeutic or diagnostic agent is mixed with the first destructive reagent using a mixer (e.g., an orbital mixer or nutating mixer) at about 60 rpm. In some embodiments, the mixture comprising the soluble therapeutic or diagnostic agent is mixed with the first destructive reagent using a mixer (e.g., an orbital mixer or nutating mixer) at about 120 rpm.

Non-limiting examples of membrane filters include silver membranes, aluminum oxide membranes, cellulose acetate membranes, ceramic membranes, glass fiber membranes, mixed cellulose esters membranes, nylon membranes, polyacrylonitrile membranes, polycarbonate membranes, polyethersulfone membranes, polyester membranes, polyethylene membranes, polypropylene membranes, polytetrafluoroethylene membranes, and polyvinylidene fluoride membranes. In some embodiments, the membrane filter is a polyvinylidene fluoride, polytetrafluoroethylene, or polyethersulfone membrane. In some embodiments, the membrane filter is a polyvinylidene fluoride membrane.

Non-limiting examples of sieves include sieve stacks, wire-mesh sieves, electroformed grid sieves, wet washing sieves, microplate sieves, air jet sieves, and sieve shakers. In some embodiments, the sieve may be a sieve known in the art such as those disclosed in USP <786> (USP <786> Particle Size Distribution Estimation by Analytical Sieving, DOI: https_doi_org/10.31003/USPNF_M99584_02_01), incorporated by reference in its entirety.

In some embodiments, the membrane filter or sieve has a pore size of from about 0.1 μm to about 1000 μm (e.g., about 0.1 μm to about 10 μm, about 0.1 μm to about 5 μm, about 0.1 μm to about 1 μm, about 0.1 μm to about 0.5 μm, about 10 μm to about 25 μm, about 25 μm to about 50 μm, about 50 μm to about 100 μm, about 100 μm to about 150 μm, about 150 μm to about 200 μm, about 200 μm to about 250 μm, about 250 μm to about 100 μm etc.). In some embodiments, a membrane filter or sieve has a pore size of about 0.65 μm. In certain embodiments, the membrane filter or sieve has a pore size of about 0.1 μm, about 0.5 μm, about 1 μm, about 2 μm, about 2.5 μm, about 5 μm, about 10 μm, about 15 μm, about 20 μm, about 25 μm, about 30 μm, about 40 μm, about 50 μm, about 60 μm, about 70 μm, about 75 μm, about 80 μm, about 90 μm, about 100 μm, about 125 μm, about 150 μm, about 175 μm, about 200 μm, about 250 μm, about 500 μm, about 750 μm, about 1000 μm, etc.

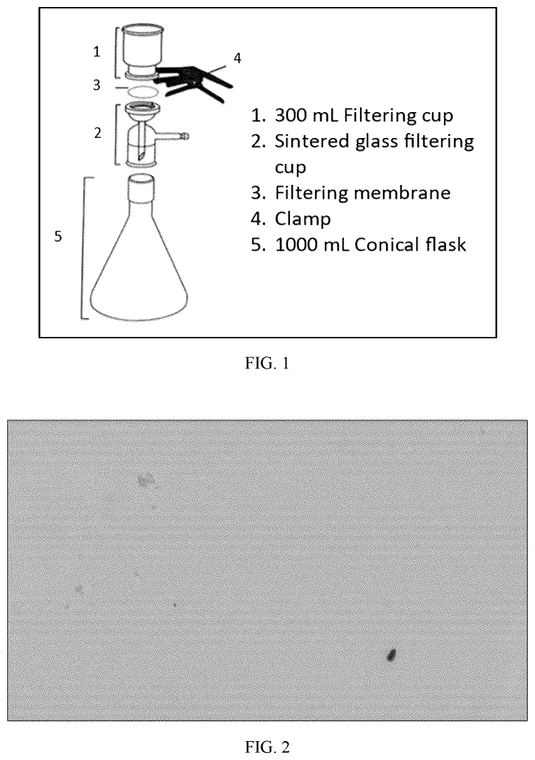

In some embodiments, filtering a mixture comprising soluble agent (e.g., therapeutic or diagnostic agent) through a filtration apparatus comprises applying a vacuum and pulling the at least one destructive reagent through the first membrane filter (e.g., a membrane filter, depth filter, or sieve of the filtration apparatus). For example, a filtration apparatus comprises a filtering cup, filtering membrane, and a conical flask. An example embodiment of a filtration apparatus is illustrated in FIG. 1.

In some embodiments, filtering a mixture comprising soluble agent (e.g., therapeutic or diagnostic agent) through a filtration apparatus further comprises contacting soluble agent with a destructive reagent (e.g., a second destructive agent). Contacting soluble agent with a destructive reagent may be achieved by adding destructive reagent to a filtration apparatus. For example, agents on a membrane filter or sieve of a filtration apparatus are contacted with a destructive reagent (e.g., 4 M urea, 10 mM TCEP) when the destructive reagent is added to the filtration apparatus.

In some embodiments, adding at least one destructive reagent to a mixture comprising the soluble therapeutic or diagnostic agent further comprises contacting a second destructive reagent with the soluble therapeutic or diagnostic agent. In some embodiments, contacting a second destructive reagent with the soluble therapeutic or diagnostic agent comprises adding the second destructive agent to the mixture comprising the soluble therapeutic or diagnostic agent and the first destructive reagent.

In other embodiments, adding at least one destructive reagent to a mixture comprising the soluble therapeutic or diagnostic agent further comprises adding a second destructive reagent to a mixture comprising the soluble therapeutic or diagnostic agent and a first destructive reagent. In some embodiments, the second destructive reagent added to the mixture has a concentration of from about 0.1 mM to about 1000 mM (e.g., about 0.1 mM to about 900 mM, about 0.1 mM to about 800 mM, about 0.1 mM to about 700 mM, about 0.1 mM to about 600 mM, about 0.1 mM to about 500 mM, about 0.1 mM to about 400 mM, about 0.1 mM to about 300 mM, about 0.1 mM to about 200 mM, about 0.1 mM to about 100 mM, about 1 mM to about 100 mM, about 2 mM to about 100 mM, about 3 mM to about 100 mM, about 4 mM to about 100 mM, about 5 mM to about 100 mM, etc.). In some embodiments, the second destructive reagent added to the mixture has a concentration of from about 5 mM to about 100 mM. In some embodiments, the concentration of the second destructive reagent is about 15 mM.

In some embodiments, the destructive reagent (e.g., a second destructive agent) is contacted with the soluble agent (e.g., therapeutic or diagnostic agent) at a temperature of from about 20° C. to about 90° C. (e.g., about 20° C. to about 80° C., about 20° C. to about 70° C., about 20° C. to about 60° C., about 20° C. to about 50° C., about 20° C. to about 40° C., about 20° C. to about 30° C., about 20° C. to about 25° C., etc.). In some embodiments, the second destructive reagent is contacted with the soluble therapeutic or diagnostic agent at a temperature of from about 20° C. to about 25° C.

In some embodiments, a second destructive reagent comprises a reducing agent (e.g., phosphine) such as iodoacetamide (IAM), triphenylphosphine, tributylphosphine, trihydroxymethylphosphine, trihydroxypropylphosphine, triscarboethoxy-phosphine (TCEP), or combinations thereof. In some embodiments, a second destructive reagent comprises TCEP and urea. In other embodiments, a second destructive reagent comprises IAM, TCEP, or a combination thereof. In some embodiments, a second destructive reagent comprises TCEP. In other embodiments, a second destructive reagent is IAM. A destructive reagent may be prepared by filtering a solution comprising a destructive reagent (e.g., denaturant) through a membrane filter (e.g., a polyvinylidene fluoride membrane, polytetrafluoroethylene membrane, polyethersulfone membrane).

In some embodiments, a membrane filter (e.g., a second membrane filter) for preparing a destructive reagent has a pore size of from about 0.1 μm to about 10 μm (e.g., about 0.1 μm to about 5 μm, about 0.1 μm to about 1 μm, about 0.1 μm to about 0.5 μm, etc.). In some embodiments, the membrane filter has a pore size of from about 0.1 μm to about 0.5 μm. In some embodiments, the membrane filter has a pore size of about 0.22 μm.

In some embodiments, a destructive reagent (e.g., a second protein destructive agent) is contacted (e.g., incubated) with soluble agent (e.g., soluble therapeutic or diagnostic agent) for a time period of from about 1 minute to about 3 days (e.g., about 1 minute to about 2 days, about 1 minute to about 24 hours, about 1 minute to about 12 hours, about 1 minute to about 5 hours, about 1 minute to about 4 hours, about 1 minute to about 3 hours, about 1 minute to about 2 hours, about 1 minute to about 1 hour, about 1 minute to about 30 minutes, etc.). In some embodiments, a second destructive reagent is contacted with the soluble therapeutic or diagnostic agent for about 20 minutes.

In some embodiments, methods of the present disclosure further comprise adding at least one enzyme to a mixture comprising a soluble therapeutic or diagnostic agent and at least one destructive reagent to digest the soluble therapeutic or diagnostic agent. In some embodiments, the at least one enzyme is an endoprotease, exoprotease, trypsin, chymotrypsin, pepsin, caspases, papain, elastase, thermolysin, renin, matrix metalloproteases (MMPs), or a combination thereof. In some embodiments, the at least one enzyme is an endoprotease. In some embodiments, the at least one enzyme is lysyl endopeptidase, trypsin, or a combination thereof.

In other embodiments, methods of the present disclosure further comprises adding a first enzyme to a mixture comprising the soluble therapeutic or diagnostic agent and the at least one destructive reagent, decreasing the concentration of the first destructive agent in the mixture, and adding a second enzyme to the mixture. In some embodiments, the first enzyme is an endoprotease. In some embodiments, the first enzyme is lysyl endopeptidase. In some embodiments, the second enzyme is trypsin.

In some embodiments, adding a first enzyme to the mixture comprises contacting the first enzyme with the soluble therapeutic or diagnostic agent for from about 30 minutes to about 2 hours. In some embodiments, the first enzyme is contacted with the soluble therapeutic or diagnostic agent for about 1 hour.

In some embodiments, the concentration of the first destructive agent is decreased to less than about 5.0 M (e.g., less than about 4.0 M, less than about 3.0 M, less than about 2.0 M, less than about 1.0 M, etc.) in the mixture. In some embodiments, the concentration of the first destructive agent is decreased to less than about 1.6 M in the mixture.

In some embodiments, the concentration of the first destructive agent is decreased to from about 0 M to about 5.0 M (e.g., about 0 M to about 4.0 M, about 0 M to about 3.0 M, about 0 M to about 2.0 M, about 0 M to about 1.9 M, about 0 M to about 1.8 M, about 0 M to about 1.7 M, about 0 M to about 1.6 M, etc.) in the mixture. In some embodiments, the concentration of the first destructive agent is decreased to from about 0 M to about 1.6 M.

In some embodiments, adding a second enzyme to the mixture comprises contacting the second enzyme with the soluble therapeutic or diagnostic agent for from about 1 hour to about 5 hours. In some embodiments, the second enzyme is contacted with the soluble therapeutic or diagnostic agent for about 3 hours.

The present disclosure provides, in some embodiments, methods of detecting particulate contaminants in particles or a composition comprising particles, wherein the particles comprise a therapeutic or diagnostic agent, and wherein the particulate contaminants do not comprise the therapeutic or diagnostic agent, the methods comprising:
a) mixing the particles or the composition comprising particles with a dissolution media under conditions in which the particles dissolve to produce a mixture comprising soluble therapeutic or diagnostic agent;
b) adding at least one enzyme to the mixture comprising the soluble therapeutic or diagnostic agent to digest the therapeutic or diagnostic agent;
c) filtering the mixture through a filtration apparatus comprising a membrane filter or a sieve; and
d) inspecting the surface of the membrane filter or the sieve for the presence of particulate contaminants.

In some embodiments, disclosed herein are methods of detecting particulate contaminants in particles or a composition comprising particles, wherein the particles comprise a therapeutic or diagnostic agent, and wherein the particulate contaminants do not comprise the therapeutic or diagnostic agent, the methods comprising:
a) mixing the particles or the composition comprising particles with a dissolution media under conditions in which the particles dissolve to produce a mixture comprising soluble therapeutic or diagnostic agent;
b) adding at least one enzyme to the mixture comprising the soluble therapeutic or diagnostic agent to digest the therapeutic or diagnostic agent;
c) filtering the mixture through a filtration apparatus comprising a first membrane filter; and
d) inspecting the surface of the first membrane filter for the presence of particulate contaminants.

Also disclosed herein, in some embodiments, are methods of detecting particulate contaminants in particles or a composition comprising particles, wherein the particles comprise a therapeutic or diagnostic agent, and wherein the particulate contaminants do not comprise the therapeutic or diagnostic agent, the method comprising:
a) filtering the particles or the composition comprising particles through a filtration apparatus comprising a membrane filter (e.g., a first membrane filter) or a sieve (e.g., 60 μm sieve) to produce a filtrate comprising the particles; and
b) inspecting (e.g., visually inspecting) the surface of the membrane filter or the sieve for the presence of particulate contaminants (e.g., visible particulate contaminants).

In some embodiments, the particulate contaminants are visible particulate contaminants (e.g., fibers, rubber particles), subvisible particulate contaminants, or a combination thereof. In some embodiments, the particulate contaminants are visible particulate contaminants. In some embodiments, the particulate contaminants are subvisible particulate contaminants.

In some embodiments, methods of the present disclosure comprises detecting particulate contaminants in a composition comprising particles, wherein the composition comprises particles suspended in a liquid.

In some embodiments, methods of the present disclosure further comprise adding at least one detergent to the mixture comprising the soluble therapeutic or diagnostic agent and the at least one enzyme. In some embodiments, the at least one detergent is sodium lauryl sulfate, octylphenol ethoxylate, polyoxyethylene (20) sorbitan monolaurate (Tween 20), 3-[(3-cholamidopropyl)dimethylammonio]-1-propanesulfonate (CHAPS), octylphenoxypolyethoxyethanol (Triton X-100), n-octyl-β-D-glucopyranoside, or a combination thereof.

In some embodiments, filtering a mixture comprising soluble agent (e.g., therapeutic or diagnostic agent) through a filtration apparatus further comprises applying vacuum to pull a destructive reagent through a filter. Further reagents (e.g., water) may be added onto the filter while continuing to apply the vacuum to ensure complete removal of residual destructive reagent.

In some embodiments, methods of the present disclosure further comprise drying a membrane filter (e.g., a first membrane filter) or a sieve. In some embodiments, the membrane filter or the sieve is dried before inspecting the surface of the membrane filter. For example, drying a membrane filter or the sieve may comprise adding a liquid-removing reagent to a membrane filter. The liquid may be an aqueous or non-aqueous liquid. In some embodiments, the non-aqueous liquid comprises ethyl oleate, propylene glycol diesters, or a combination thereof. In some embodiments, the liquid-removing reagent is a solvent such as but not limited to simple alcohols such as methanol, ethanol, isopropyl alcohol, octanol, hexanol, decanol, propanol, butanol, and a combination thereof. In some embodiments, the liquid-removing reagent comprises methanol, ethanol, and isopropanol. In some embodiments, the liquid-removing reagent is an alcohol (e.g., reagent alcohol).

In some embodiments, drying a membrane filter (e.g., a first membrane filter) or a sieve further comprises applying a vacuum and pulling a liquid-removing reagent through the membrane filter or the sieve.

In some embodiments, drying a membrane filter (e.g., a first membrane filter) or a sieve further comprises removing the membrane filter or the sieve from the filtration apparatus and drying the membrane filter or the sieve. Suitable methods of drying the membrane filter or the sieve are well known to those skilled in the art but include, but are not limited to, include air-drying, freeze-drying, vacuum drying optionally at elevated humidity, microwave vacuum drying, supercritical processing (such as RES, SEDS, etc.), forced air drying, and the like. In some embodiments, the membrane filter or the sieve is dried for a time period of from about 1 minute to about 5 hours (e.g., about 1 minute to about 4 hours, about 1 minute to about 3 hours, about 1 minute to about 2 hours, about 1 minute to about 1 hour, about 1 minute to about 30 minutes, etc.). In some embodiments, the membrane filter or the sieve is dried for at least about 15 minutes. In some embodiments, the membrane filter or the sieve is dried for about 20 minutes.

In some embodiments, inspecting the surface of a membrane filter (e.g., a first membrane filter) or a sieve for the presence of particulate contaminants comprises using an optical instrument (e.g., a microscope) to detect the particulate contaminants. In some embodiments, an automated microscope is used to detect the particulate contaminants.

In some embodiments, inspecting the surface of a membrane filter (e.g., a first membrane filter) or a sieve for the presence of particulate contaminants comprises using visual inspection and/or manually counting the number of particulate contaminants.

In some embodiments, particulate contaminants (e.g., extrinsic and insoluble intrinsic contaminants) in particles or a composition comprising particles are isolated using methods of the present disclosure and imaged using a microscope to size and count the contaminants as per USP <788>, USP <1788>, USP <790>, and USP <1790>. The particles may be dissolved and treated to remove any protein particles intrinsic to the drug product. The sample may then be filtered through a membrane (e.g., a 0.65 μm membrane) or a sieve to isolate any contaminants larger than 10 μm and imaged using wide-field imaging or any other appropriate imaging type (e.g., using a microscope with polarized light) and an automated stage capable of imaging a large cross-sectional area. In some embodiments, automated counting is performed. The image captured may then be processed and segmented to size and count particles and reported in (e.g., compendial) bin sizes of, for example, 10, 25, 100, 150, 250, and 1000 μm. In some embodiments, manual counting is performed. In some embodiments, manual counting is performed as per USP <788> and USP <790> which utilize a manual approach based on an operator.

In some embodiments, the particles are filtered through a membrane or a sieve (e.g., 50 μm sieve), dissolved with a dissolution media, and imaged to determine the size and count of particles remaining (e.g., using automated or manual counting approaches).

The present disclosure also relates to methods of dissolving particles (e.g., protein particles). In some embodiments, the particles comprise an agent (e.g., a therapeutic or diagnostic agent). In some embodiments, methods of dissolving particles comprise one or more of: a) adding a dissolution media to particles or a composition comprising particles suspended in a liquid; and b) mixing the dissolution media with the particles or composition, thereby dissolving the particles to produce a solution comprising soluble agent (e.g., a therapeutic or diagnostic agent). In some embodiments, the dissolution media comprises non-ionic surfactant (e.g., 0.5% v/v non-ionic surfactant in 1× phosphate buffered saline (PBS) at a pH of about 7.4). In certain embodiments, the composition comprises particles suspended in a liquid.

The present disclosure also relates to methods of preparing a destructive reagent, comprising one or more of: a) mixing a denaturant with a liquid to produce a first solution comprising a denaturant; b) mixing a reductant with a liquid to produce a second solution comprising a reductant; and c) filtering the first and second solutions using a filtration apparatus, thereby obtaining a destructive reagent. In some embodiments, the liquid comprises class 1 ultrapure water. In some embodiments, the filtering comprises vacuum filtering a solution comprising a denaturant followed by vacuum filtering a solution comprising a reductant. In some embodiments, the reductant comprises tris(2-carboxyethyl) phosphine hydrochloride, beta-mercaptoethanol, dithiotheriol, or a combination thereof. In some embodiments, the reductant comprises tris(2-carboxyethyl) phosphine hydrochloride.

In some embodiments, methods of preparing a destructive reagent comprise one or more of: a) mixing a first chemical agent (e.g., a first denaturant) with a second chemical agent (e.g., a second denaturant) to produce a first solution; b) adding a reductant to the first solution to produce a second solution comprising the reductant; and c) filtering the second solution using a filtration apparatus, thereby obtaining a destructive reagent. In some embodiments, mixing a first chemical agent with a second chemical agent comprises using a nutating mixer for about 10 minutes.

In some embodiments, methods of preparing a destructive reagent comprise one or more of: a) mixing a first denaturant with a second denaturant to produce a first solution; b) adding a reductant to the first solution to produce a second solution comprising the reductant; and c) filtering the second solution using a filtration apparatus, thereby obtaining a destructive reagent. In some embodiments, the first denaturant comprises urea. In some embodiments, the second denaturant comprises sodium dodecyl sulfate. In some embodiments, mixing a first denaturant with a second denaturant comprises using a nutating mixer for about 10 minutes. In some embodiments, the reductant comprises tris(2-carboxyethyl) phosphine hydrochloride, beta-mercaptoethanol, dithiotheriol, or a combination thereof. In some embodiments, the reductant comprises tris(2-carboxyethyl) phosphine hydrochloride.

In some embodiments, mixing a denaturant with a liquid or mixing a first denaturant with a second denaturant comprises using a mixer (e.g., rotators, shakers, rockers, combinations thereof, etc.). Non-limiting example of mixers include nutating mixers and orbital mixers. In some embodiments, the mixing is performed at a speed of from about 10 revolutions per minute (rpm) to about 200 rpm (e.g., about 10 rpm to about 150 rpm, about 10 rpm to about 100 rpm, about 50 rpm to about 100 rpm, etc.). In some embodiments, the mixing is performed at about 60 rpm.

In some embodiments, mixing a denaturant with a liquid or mixing a first denaturant with a second denaturant is performed for a time period of from about 1 minute to about 5 hours (e.g., about 1 minute to about 4 hours, about 1 minute to about 3 hours, about 1 minute to about 2 hours, about 1 minute to about 60 minutes, etc.). In some embodiments, the denaturant is mixed with the liquid for about 30 minutes. In some embodiments, a first denaturant is mixed with a second denaturant for about 10 minutes.

In some embodiments, methods of detecting particulate contaminants described herein are used in the manufacture of particles (e.g., protein particles) or a pharmaceutical composition (e.g., a drug product) comprising particles, wherein the particles comprise a therapeutic or diagnostic agent (e.g., a therapeutic biologic or salt thereof). In certain embodiments, the methods described herein further comprise determining whether to release (or reject for non-conformance) a composition for administration to a subject (e.g., a human subject). In some embodiments, determining whether to release a composition for administration to a subject involves qualitative and/or quantitative analysis of the particulate contaminants and comparison of those observations against pre-identified specifications.

Particles of the present disclosure may be produced using various techniques. For example, the generation of particles can be accomplished by producing a droplet of a liquid comprising an active agent dissolved in a solvent. The solvent can then be extracted from the droplets by depositing the droplets into a liquid in which the solvent, but not the active agent, is soluble leaving behind a solid particle. Isolation of the particles occur following evaporation of the liquids.

In some embodiments, a particle comprises an agent or a composition comprises a plurality of particles comprising an agent, wherein the particle or the plurality of particles comprises less than about 25% internal void spaces and the circularity of the particle is from about 0.10 to about 1.00. In certain embodiments, the particle or plurality of particles comprise about 0.1%, about 1%, about 5%, about 10%, about 15%, about 20%, or about 25% internal void spaces on a weight-volume average basis. In some embodiments, the composition comprises particles suspended in a liquid.

In some embodiments, particles of the present disclosure can be formed by creating droplets of a first liquid, e.g., including an agent, and removing the first liquid, e.g., through its dispersal in a second liquid and/or evaporation, to solidify the droplets. In some embodiments, the particles may be stored for extended periods of time without significant loss of activity or the need for refrigeration. These particles may be used to generate stabilized pharmaceutical compositions, pharmaceutical suspension formulations, pharmaceutical powder formulations (e.g., inhalable powders, injectable powders), creams or other topical pastes, nutraceuticals, or cosmetics. The term “pharmaceutical composition” as used herein, denotes a composition in which a therapeutic or diagnostic agent retains, or partially retains, its intended biological activity or functional form, and in which only pharmaceutically acceptable components are included.

Other methods of treatment of therapeutic or diagnostic agents post-dissolution of particles are contemplated herein, including treatment with acids and bases, usage of organic compounds such as alcohols, and metal salts (e.g., mercury, lead, etc.)

It will be readily understood that the aspects and embodiments, as generally described herein, are exemplary. The following more detailed description of various aspects and embodiments are not intended to limit the scope of the present disclosure, but is merely representative of various aspects and embodiments. Moreover, the compositions and methods disclosed herein may be changed by those skilled in the art without departing from the scope of the present disclosure. Unless defined otherwise, all technical and scientific terms used herein have the same meaning as is commonly understood by one of skill in the art to which this disclosure belongs. All publications and patents referred to herein are incorporated by reference.

### Definitions

For purposes of the present disclosure, the following definitions will be used unless expressly stated otherwise:

The terms “a”, “an”, “the” and similar referents used in the context of describing the present disclosure are to be construed to cover both the singular and the plural, unless otherwise indicated herein or clearly contradicted by context. All methods described herein, can be performed in any suitable order unless otherwise indicated herein or otherwise clearly contradicted by context. The use of any and all examples, or exemplary language (e.g., “such as”) provided herein, is intended merely to better illuminate the present disclosure and does not pose a limitation on the scope of the disclosure otherwise claimed. No language in the present specification should be construed as indicating any unclaimed element is essential to the practice of the disclosure.

The term “about” in relation to a given numerical value, such as for temperature and period of time, is meant to include numerical values within 10% of the specified value.

As used herein, an “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, iso-butyl, sec-butyl, tert-butyl, n-pentyl, tert-pentyl, neo-pentyl, iso-pentyl, sec-pentyl, 3-pentyl, sec-iso-pentyl, active-pentyl, hexyl, heptyl, octyl, ethylhexyl, and the like. A C₁₋₈ straight chained or branched alkyl group is also referred to as a “lower alkyl” group. An alkyl group with two open valences is sometimes referred to as an alkylene group, such as methylene, ethylene, propylene and the like. Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, an alkyl, a halogen, a hydroxyl, a carbonyl (such as a carboxyl, and alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamide, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN and the like. In other embodiments, the term “alkyl” can mean “cycloalkyl” which refers to a non-aromatic carbocyclic ring having 3 to 10 carbon ring atoms, which are carbon atoms bound together to form the ring. The ring may be saturated or have one or more carbon-carbon double bonds. Examples of cycloalkyl include, but not limited to cyclopropyl, cyclobutyl, cyclopentyl, cyclopentenyl, cyclohexyl, cyclohexenyl, and cycloheptyl, as well as bridged and caged saturated ring groups such as norbornyl and adamantyl. As described herein, organic solvents include, but are not limited to aliphatic hydrocarbon solvents, aromatic hydrocarbon solvents, alcohols or alkylalcohols, alkylethers, sulfoxides, alkylketones, alkylacetates, trialkylamines, alkylformates, trialkylamines, or a combination thereof. Aliphatic hydrocarbon solvents can be pentane, hexane, heptane, octane, cyclohexane, and the like or a combination thereof. Aromatic hydrocarbon solvents can be benzene, toluene, and the like or a combination thereof. Alcohols or alkylalcohols include, for example, methanol, ethanol, propanol, butanol, pentanol, hexanol, heptanol, octanol, decanol, amylalcohol, or a combination thereof. Alkylethers include methyl, ethyl, propyl, butyl, and the like, e.g., diethylether, diisopropylether or a combination thereof. Sulfoxides include dimethyl sulfoxide (DMSO), decylmethyl sulfoxide, tetradecylmethyl sulfoxide, and the like or a combination thereof. The term “alkylketone” refers to a ketone substituted with an alkyl group, e.g., acetone, ethylmethylketone, and the like or a combination thereof. The term “alkylacetate” refers to an acetate substituted with an alkyl group, e.g., ethylacetate, propylacetate (n-propylacetate, iso-propylacetate), butylacetate (n-butylacetate, iso-butylacetate, sec-butylacetate, tert-butylacetate), amylacetate (n-pentylacetate, tert-pentylacetate, neo-pentylacetate, iso-pentylacetate, sec-pentylacetate, 3-pentylacetate, sec-iso-pentylacetate, active-pentylacetate), 2-ethylhexylacetate, and the like or a combination thereof. The term “alkylformate” refers to a formate substituted with an alkyl group, e.g., methylformate, ethylformate, propylformate, butylformate, and the like or a combination thereof. The term “trialkylamine” refers to an amino group substituted with three alkyl groups, e.g., triethylamine.

As used herein, an “amino acid” or “residue” refers to any naturally or non-naturally occurring amino acid, any amino acid derivative or any amino acid mimic known in the art. Included are the L- as well as the D-forms of the respective amino acids, although the L-forms are usually preferred. In some embodiments, the term relates to any one of the 20 naturally occurring amino acids: glycine (Gly), alanine (Ala), valine (Val), leucine (Leu), isoleucine (Ile), proline (Pro), cysteine (Cys), methionine (Met), serine (Ser), threonine (Thr), glutamine (Gin), asparagine (Asn), glutamic acid (Glu), aspartic acid (Asp), lysine (Lys), histidine (His), arginine (Arg), phenylalanine (Phe), tryptophan (Trp), and tyrosine (Tyr) in their L-form. In certain embodiments, the amino acid side-chain may be a side-chain of Gly, Ala, Val, Leu, Ile, Met, Cys, Ser, Thr, Trp, Phe, Lys, Arg, His, Tyr, Asn, Gln, Asp, Glu, or Pro.

As used herein, except where the context requires otherwise, the term “comprise” and variations of the term, such as “comprising”, “comprises” and “comprised”, are not intended to exclude further additives, components, integers or steps. The terms “including” and “comprising” may be used interchangeably. As used herein, the phrases “selected from the group consisting of”, “chosen from”, and the like, include mixtures of the specified materials. Where a numerical limit or range is stated herein, the endpoints are included. Also, all values and subranges within a numerical limit or range are specifically included as if explicitly written out. References to an element in the singular is not intended to mean “one and only one” unless specifically stated, but rather “one or more.” Unless specifically stated otherwise, terms such as “some” refer to one or more, and singular terms such as “a”, “an” and “the” refer to one or more.

The term “oligopeptide” is used to refer to a peptide with fewer members of amino acids as opposed to a polypeptide or protein. Oligopeptides described herein, are typically comprised of about two to about forty amino acid residues. Oligopeptides include dipeptides (two amino acids), tripeptides (three amino acids), tetrapeptides (four amino acids), pentapeptides (five amino acids), hexapeptides (six amino acids), heptapeptides (seven amino acids), octapeptides (eight amino acids), nonapeptides (nine amino acids), decapeptides (ten amino acids), undecapeptides (eleven amino acids), dodecapeptides (twelve amino acids), icosapeptides (twenty amino acids), tricontapeptides (thirty amino acids), tetracontapeptides (forty amino acids), etc. Oligopeptides may also be classified according to molecular structure: aeruginosas, cyanopeptolins, microcystins, microviridins, microginins, anabaenopeptins and cyclamides, etc. Homo-oligopeptides are oligopeptides comprising the same amino acid. In preferred embodiments, homo-oligopeptides comprise 10 amino acid poly-valine, poly-alanine, and poly-glycine hexamers.

The meaning of the term “peptides” are defined as small proteins of two or more amino acids linked by the carboxyl group of one to the amino group of another. Accordingly, at its basic level, peptide synthesis of whatever type comprises the repeated steps of adding amino acid or peptide molecules to one another or to an existing peptide chain. The term “peptide” generally has from about 2 to about 100 amino acids, whereas a polypeptide or protein has about 100 or more amino acids, up to a full length sequence which may be translated from a gene. Additionally, as used herein, a peptide can be a subsequence or a portion of a polypeptide or protein. In certain embodiments, the peptide consists of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues. In preferred embodiments, the peptide is from between about 30 to about 100 amino acids in length. In some embodiments, the peptide is from between about 40 to about 100 amino acids in length.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the term “prodrug” is intended to encompass therapeutic biologics which, under physiologic conditions, are converted into the therapeutically active biologics of the present disclosure. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present disclosure. In certain embodiments, some or all of the molecules in a composition represented above can be replaced with the corresponding suitable prodrug, e.g., wherein a hydroxyl in the parent molecule is presented as an ester or a carbonate or carboxylic acid present in the parent therapeutic biologic is presented as an ester.

The meaning of the term “protein” is defined as a linear polymer built from about 20 different amino acids. The type and the sequence of amino acids in a protein are specified by the DNA that produces them. In certain embodiments, the sequences can be natural and unnatural. The sequence of amino acids determines the overall structure and function of a protein. In some embodiments, proteins can contain 50 or more residues. In preferred embodiments, proteins can contain greater than about 101 residues in length. A protein's net charge can be determined by two factors: 1) the total count of acidic amino acids vs. basic amino acids; and 2) the specific solvent pH surroundings, which expose positive or negative residues. As used herein, “net positively or net negatively charged proteins” are proteins that, under non-denaturing pH surroundings, have a net positive or net negative electric charge. In general, those skilled in the art will recognize that all proteins may be considered “net negatively charged proteins”, regardless of their amino acid composition, depending on their pH and/or solvent surroundings. For example, different solvents can expose negative or positive side chains depending on the solvent pH. Proteins or peptides are preferably selected from any type of enzyme or antibodies or fragments thereof showing substantially the same activity as the corresponding enzyme or antibody. Proteins or peptides may serve as a structural material (e.g. keratin), as enzymes, as hormones, as transporters (e.g. hemoglobin), as antibodies, or as regulators of gene expression. Proteins or peptides are required for the structure, function, and regulation of cells, tissues, and organs.

The term “substantially” as used herein, refers to a majority of, or mostly, as in at least about 50%, 60%, 70%, 80%, 90%, 95%, 96%, 97%, 98%, 99%, 99.5%, 99.9%, 99.99%, or at least about 99.999% or more.

It is understood that the specific order or hierarchy of steps in the methods or processes disclosed is an illustration of exemplary approaches. Based upon design preferences, it is understood that the specific order or hierarchy of steps in the methods or processes may be rearranged. Some of the steps may be performed simultaneously. The accompanying methods claims present elements of the various steps in a sample order, and are not meant to be limited to a specific hierarchy or order presented. A phrase such as “embodiment” does not imply that such embodiment applies to all configurations of the subject technology. A disclosure relating to an embodiment may apply to all embodiments, or one or more embodiments. A phrase such as an embodiment may refer to one or more embodiments and vice-versa.

### Particles

Unless otherwise defined, all terms of art, notations and other scientific terminology used herein, are intended to have the meanings commonly understood by those of skill in the art to which this disclosure pertains. In some cases, terms with commonly understood meanings are defined herein, for clarity and/or for ready reference, and the inclusion of such definitions herein, should not necessarily be construed to represent a substantial difference over what is generally understood in the art. The techniques and procedures described or referenced herein, are generally well understood and commonly employed using conventional methodology by those skilled in the art. As appropriate, procedures involving the use of commercially available kits and reagents are generally carried out in accordance with manufacturer defined protocols and/or parameters unless otherwise noted.

In some aspects, the disclosure relates to a particle comprising an agent. In some embodiments, the particle comprising the agent comprises less than about 25% internal void spaces and the circularity of the particle is from about 0.10 to about 1.00.

The terms “particle” or “particles” or “microparticle” or “microparticles” are used herein, interchangeably in the broadest sense, refers to a discrete body or bodies. The particles described herein, are circular, spheroidal and of controlled dispersity with a characteristic size from sub-micrometers to tens of micrometers, in contrast to, e.g., a porous monolithic “cake”, which is typically produced during conventional lyophilization. This morphology allows for a flowable powder (as described by low Hausner ratios) without post-processing. In some embodiments, the term “particle” refers to a quantity of an agent or agents which is either in a state of matter that is substantially solid as compared to a liquid droplet or in a gel form. In other embodiments, the particle may include a core and a shell, where the shell may be viewed as an encapsulant. In still other embodiments, the particle does not include a shell, in which case, the particle is made up entirely of a core. The term “proto-particle” refers to a stage of particle formation in which one or more of the components comprising the particle are in an at least a partial state of desiccation. The total liquid content of the proto-particle is less than that of the droplet and greater than that of the formed particle. Similarly, the average concentration of the solutes is higher than that of the drop but typically less than that of the formed particle. The term “encapsulant” refers to a substance that can be dried or gelled around a particle core to form a shell.

As disclosed herein, the agent may be a therapeutic or diagnostic agent. Exemplary therapeutic or diagnostic agents include, but are not limited to nucleic acids, oligonucleotides, antibodies or fragment thereof, amino acids, peptides, proteins, cells, bacteria, gene therapeutics, genome engineering therapeutics, epigenome engineering therapeutics, carbohydrates, chemical drugs, contrast agents, magnetic particles, polymer beads, metal nanoparticles, metal microparticles, quantum dots, antioxidants, antibiotic agents, hormones, nucleoproteins, polysaccharides, glycoproteins, lipoproteins, steroids, analgesics, local anesthetics, anti-inflammatory agents, anti-microbial agents, chemotherapeutic agents, exosomes, outer membrane vesicles, vaccines, viruses, bacteriophages, adjuvants, vitamins, minerals, organelles, or a combination thereof. In preferred embodiments, the therapeutic agent is a therapeutic biologic or a salt thereof. Therapeutic and diagnostic agents may have a molecular weight of about 20 to about 200 kDa, e.g., about 40 to about 150 kDa, or about 50 to about 100 kDa. Table 1 provides a list of therapeutic and diagnostic agents and the typical concentration range for the general class of compound in a pharmaceutical composition.

TABLE 1

| Therapeutic/ | |
|---|---|
| diagnostic agent | Concentration range (mg/mL) |
| proteins | 20-1500 (e.g., 20-600) (or crystalline |
| | density, if higher) |
| peptides | 20-1500 (e.g., 20-600) (or crystalline |
| | density, if higher) |
| chemical drugs | 0.0001-2000 (e.g., 0.0001-1000) (or |
| | crystalline density, if higher) |
| magnetic particles | 0.001-5400 (e.g., 0.001-500) (iron oxide |
| | density) |
| carbohydrates | 0.001-400 |
| nucleic acids | 0.001-100 |

In other embodiments, the particles may include, but are not limited to, agents such as silica, titania, metals or other elements, metal salts, metal oxides, metal nitrides, metal sulfides, metal alkoxides, and/or polymers.

A therapeutic biologic, also known as a biologic medical product, or biopharmaceutical, is any pharmaceutical drug product manufactured in, extracted from, or semisynthesized from biological sources. Therapeutic biologics can include a wide range of products such as vaccines, blood and blood components, allergenics, somatic cells, gene therapy, tissues, and recombinant therapeutic proteins. In some embodiments, the biologics can be composed of sugars, proteins, or nucleic acids or complex combinations of these substances, or may be living entities such as cells and tissues. Biologics can be isolated from a variety of natural sources, e.g., a human, animal, or microorganism, and may be produced by biotechnology methods or other technologies. Gene-based and cellular biologics, for example, are often used to treat a variety of medical conditions for which no other treatments are available. In preferred embodiments of the disclosure, the therapeutic biologic is an antibody.

The terms “antibody” and “immunoglobulin” are used interchangeably in the broadest sense and include monoclonal antibodies, polyclonal antibodies, multivalent antibodies, and multispecific antibodies, regardless of how they are produced (i.e., using immunization, recombinant, synthetic methodologies). Antibodies can be gamma globulin proteins that are found in blood, or other bodily fluids of vertebrates that function in the immune system to bind antigen, hence identifying and/or neutralizing foreign objects. Antibodies can be assigned to different classes or isotypes. There are five classes of immunoglobulins: IgA, IgD, IgE, IgG, and IgM, having heavy chains designated alpha, delta, epsilon, gamma, and mu, respectively. The gamma class is further divided into subclasses based on the differences in sequences and function, e.g., humans express the following subclasses: IgG1, IgG2, IgG3, IgG4, IgA1, and IgA2. In certain embodiments of the disclosure, the IgG antibody is an IgG1 antibody. In preferred embodiments of the disclosure, the IgG1 antibody is a monoclonal IgG1 antibody. The L chain from any vertebrate species can be assigned to one of two clearly distinct types, e.g., kappa and lambda, based on the amino acid sequences of their constant domains.

The recognized immunoglobulin genes include the kappa, lambda, alpha, gamma, delta, epsilon and mu constant region genes, as well as the myriad immunoglobulin variable region genes. In some embodiments, light chains are classified as either kappa or lambda. In other embodiments, heavy chains are classified as gamma, mu, alpha, delta, or epsilon, which in turn define the immunoglobulin classes, IgG, IgM, IgA, IgD and IgE, respectively. In preferred embodiments of the disclosure, the antibody is an IgG antibody.

An exemplary antibody (immunoglobulin) structural unit comprises a tetramer. Each tetramer is composed of two identical pairs of polypeptide chains, each pair having one “light” (about sss25 kD) and one “heavy” chain (about 50-70 kD). The N-terminus of each chain defines a variable region of about 100 to 110 or more amino acids primarily responsible for antigen recognition. The terms “variable light” chain, domain, region and component are used interchangeably, are abbreviated by “VL” or “V_{L}” and refer to the light chain of an antibody or antibody fragment. Similarly, terms “variable heavy” chain, domain, region and component are used interchangeably, are abbreviated by “VH” or “V_{H}” and refer to the heavy chain of an antibody or antibody fragment. Antibodies are generally a heterotetrameric glycoprotein composed of two identical light (L) chains and two identical heavy (H) chains. Each L chain is linked to a H chain by one covalent disulfide bond. The two H chains are linked to each other by one or more disulfide bonds depending on the H chain isotype. Each H and L chain also has regularly spaced intra-chain disulfide bridges. H and L chains define specific Ig domains. In particular, each H chain has at the N-terminus, a variable domain (VH) followed by three constant domains (CH) for each of the alpha and gamma chains and four CH domains for p and c isotypes. Each L chain has at the N-terminus, a variable domain (VL) followed by a constant domain (CL) at its other end. The VL is aligned with the VH and the CL is aligned with the first constant domain of the heavy chain (CHL). The constant domain includes the Fc portion which comprises the carboxy-terminal portions of both H chains held together by disulfides. The effector functions of antibodies such as ADCC are determined by sequences in the Fc region, which is also the part recognized by Fc receptors (FcR) found on certain types of cells.

As disclosed herein, the pairing of a VH and VL together form a “variable region” or “variable domain” including the amino-terminal domains of the heavy or light chain of the antibody. The variable domain of the heavy chain may be referred to as “VH”. The variable domain of the light chain may be referred to as “VL”. The V domain contains an “antigen binding site” which affects antigen binding and defines specificity of a particular antibody for its particular antigen. V regions span about 110 amino acid residues and consist of relatively invariant stretches called framework regions (FRs) (generally about 4) of 15-30 amino acids separated by shorter regions of extreme variability called “hypervariable regions” (generally about 3) that are each generally 9-12 amino acids long. The FRs largely adopt a p-sheet configuration and the hypervariable regions form loops connecting, and in some cases forming part of, the p-sheet structure. In certain embodiments, the “hypervariable region” refers to the regions of an antibody variable domain which are hypervariable in sequence and/or form structurally defined loops. Generally, antibodies comprise six hypervariable regions; three in the VH (H1, H2, H3), and three in the VL (L1, L2, L3). “Framework” or “FR” residues are those variable domain residues other than the hypervariable region residues defined herein.

The terms “full length antibody”, “intact antibody” and “whole antibody” are used herein, interchangeably, to refer to an antibody in its substantially intact form, not as antibody fragments as defined above. The terms particularly refer to an antibody with heavy chains that contain the Fc region. A full length antibody can be a native sequence antibody or an antibody variant. In certain embodiments, an “intact” or “whole” antibody is one which comprises an antigen-binding site as well as a CL and at least heavy chain constant domains, CH1, CH2 and CH3. The constant domains may be native sequence constant domains (e.g. human native sequence constant domains) or amino acid sequence variants thereof.

As disclosed herein, “whole antibody fragments including a variable domain” include Fab, Fab′, F(ab′)₂, and Fv fragments; diabodies; linear antibodies, single-chain antibody molecules; and multispecific antibodies formed from antibody fragments. The “Fab fragment” consists of an entire L chain along with the variable region domain of the H chain (VH), and the first constant domain of one heavy chain (CHI). Each Fab fragment is monovalent with respect to antigen binding, i.e., it has a single antigen-binding site. A “Fab′ fragment” differs from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. A “F(ab′)₂ fragment” roughly corresponds to two disulfide linked Fab fragments having divalent antigen-binding activity and is still capable of cross-linking antigen. An “Fv” is the minimum antibody fragment which contains a complete antigen-recognition and binding site. This fragment consists of a dimer of one heavy and one light chain variable region domain in tight, non-covalent association. In a single-chain Fv (scFv) species, one heavy and one light chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody. “Single-chain Fv” also abbreviated as “sFv” or “scFv” are antibody fragments that comprise the VH and VL antibody domains connected to form a single polypeptide chain. In preferred embodiments, the scFv polypeptide further comprises a polypeptide linker between the VH and VL domains which enables the scFv to form the desired structure for antigen binding. In some embodiments, a “single variable domain” is half of an Fv (comprising only three CDRs specific for an antigen) that has the ability to recognize and bind antigen, although at a lower affinity than the entire binding site.

In some embodiments, “diabodies” refer to antibody fragments with two antigen-binding sites, which fragments comprise a heavy-chain variable domain (VH) connected to a light-chain variable domain (VL) in the same polypeptide chain (VH-VL). The small antibody fragments are prepared by constructing sFv fragments with short linkers (about 5-10 residues) between the VH and VL domains such that inter-chain but not intra-chain pairing of the V domains is achieved, resulting in a bivalent fragment, i.e., fragment having two antigen-binding sites. In other embodiments, diabodies may be bivalent or bispecific. In certain embodiments, bispecific diabodies are heterodimers of two “crossover” sFv fragments in which the VH and VL domains of the two antibodies are present on different polypeptide chains. Triabodies and tetrabodies are also generally known in the art.

“Antigen binding fragments” of antibodies as described herein, comprise only a portion of an intact antibody, generally including an antigen binding site of the intact antibody and thus retaining the ability to bind antigen. Exemplary examples of antibody fragments encompassed by the present definition include but are not limited to: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment which consists of a VH domain; (vii) isolated CDR regions; (viii) F(ab′)₂ fragments, a bivalent fragment including two Fab′ fragments linked by a disulfide bridge at the hinge region; (ix) single chain antibody molecules (e.g. single chain Fv; scFv); (x) “diabodies” with two antigen binding sites, comprising a heavy chain variable domain (VH) connected to a light chain variable domain (VL) in the same polypeptide chain; (xi) “linear antibodies” comprising a pair of tandem Fd, segments (VH-CH1-VH-CH1) which, together with complementary light chain polypeptides, form a pair of antigen binding regions. In some embodiments, an “antigen binding site” generally refers to a molecule that includes at least the hypervariable and framework regions that are required for imparting antigen binding function to a V domain. An antigen binding site may be in the form of an antibody or an antibody fragment, (such as a dAb, Fab, Fd, Fv, F(ab′)₂ or scFv) in a method described herein.

In some embodiments, the term “single-chain Fv” or “scFv” or “single chain” antibody can refer to antibody fragments comprising the VH and VL domains of antibody, wherein these domains are present in a single polypeptide chain. Generally, the Fv polypeptide further comprises a polypeptide linker between the VH and VL domains which enables the sFv to form the desired structure for antigen binding. For a review of sFv, see Pluckthun, THE PHARMACOLOGY OF MONOCLONAL ANTIBODIES, vol. 113, Rosenburg and Moore eds. Springer-Verlag, New York, pp. 269-315 (1994).

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies (mAbs) are highly specific, being directed against a single antigenic site or determinant on the antigen. In addition to their specificity, the monoclonal antibodies are advantageous in that they may be synthesized uncontaminated by other antibodies. Monoclonal antibodies may be prepared by the hybridoma methodology. The monoclonal antibodies may also be isolated from phage antibody libraries using molecular engineering techniques. The monoclonal antibodies of the disclosure may be generated by recombinant DNA methods, and are sometimes referred to as “recombinant antibodies” or “recombinant monoclonal antibodies” as described herein. In some embodiments, a monoclonal antibody is a single species of antibody wherein every antibody molecule recognizes the same epitope because all antibody producing cells are derived from a single B-lymphocyte cell line. The methods for generating monoclonal antibodies (mAbs) generally begin along the same lines as those for preparing polyclonal antibodies. In other embodiments, rodents such as mice and rats are used in generating monoclonal antibodies. In certain embodiments, rabbit, sheep, or frog cells are used in generating monoclonal antibodies. The use of rats is well known and may provide certain advantages. Mice (e.g., BALB/c mice) are routinely used and generally give a high percentage of stable fusions. In still other embodiments of the disclosure, the antibody is a monoclonal antibody. In preferred embodiments of the disclosure, the IgG antibody is monoclonal.

In other embodiments, recombinant antibody fragments may be isolated from phage antibody libraries using techniques well known in the art. See, for example, Clackson et al., 1991, Nature 352:624-628; Marks et al., 1991, J. Mol. Biol. 222:581-597. Recombinant antibody fragments may be derived from large phage antibody libraries generated by recombination in bacteria (Sblattero and Bradbury, 2000, Nature Biotechnology 18:75-80; and as described herein). Polynucleotides encoding the VH and VL components of antibody fragments (i.e., scFv) may be used to generate recombinant full length immunoglobulins using methods known in the art (see, for example, Persic et al., 1997, Gene 187:9-18).

An “isolated antibody” is one that has been identified and separated and/or recovered from a component of its pre-existing environment. Contaminant components are materials that would interfere with therapeutic uses for the antibody, and may include enzymes, hormones, and other proteinaceous or nonproteinaceous solutes.

As used herein, a “human antibody” refers to an antibody that possesses an amino acid sequence that corresponds to that of an antibody produced by a human. Human antibodies can be produced using various techniques known in the art, including phage-display libraries. Human antibodies can be prepared by administering the antigen to a transgenic animal that has been modified to produce such antibodies in response to antigenic challenge, but whose endogenous loci has been disabled. “Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

An “affinity matured” antibody is one with one or more alterations in one or more hypervariable region thereof that result in an improvement in the affinity of the antibody for antigen, compared to a parent antibody that does not possess those alterations. In some embodiments, affinity matured antibodies can have micromolar affinities for the target antigen. In other embodiments, affinity matured antibodies can have nanomolar or even picomolar affinities for the target antigen. Affinity matured antibodies are produced by procedures known in the art.

A “blocking” antibody or an “antagonist” antibody is one that inhibits or reduces biological activity of the antigen it binds. In some embodiments, blocking antibodies or antagonist antibodies substantially or completely inhibit the biological activity of the antigen. An “agonist antibody”, as used herein, is an antibody, which mimics at least one of the functional activities of a polypeptide of interest.

“Binding affinity” generally refers to the strength of the sum total of noncovalent interactions between a single binding site of a molecule, e.g., an antibody, and its binding partner, e.g., an antigen. Unless indicated otherwise, as used herein, “binding affinity” refers to intrinsic binding affinity, which reflects a 1:1 interaction between members of a binding pair, e.g., antibody and antigen. The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. “Epitope” generally refers to that part of an antigen that is bound by the antigen binding site of an antibody. In some embodiments, an epitope may be “linear” in the sense that the hypervariable loops of the antibody CDRs that form the antigen binding site bind to a sequence of amino acids as in a primary protein structure. In other embodiments, the epitope is a “conformational epitope”, i.e. one in which the hypervariable loops of the CDRs bind to residues as they are presented in the tertiary or quaternary protein structure.

In some embodiments of any of the foregoing composition of matter and methods, the therapeutic biologic is an antibody. In other embodiments, the antibody includes but are not limited to 3F8, Abagovomab, Abatacept, Abciximab, Abituzumab, Abrezekimab, Abrilumab, Acritumomab, Actoxumab, Abituzumab, Adalimumab-adbm, Adalimumab-atto, Adalimumab-bwwb, Adecatumumab, Ado-trastuzumab emtansine, Aducanumab, Afasevikumab, Afelimomab, Aflibercept, Afutuzumab, Alacizumab pegol, ALD518, Alefacept, Alemtuzumab, Alirocumab, Altumomab pentetate, Amatuximab, Anatumomab mafenatox, Andecaliximab, Anetumab ravtansine, Anifrolumab, Anrukinzumab, Apolizumab, Aprutumab ixadotin, Arcitumomab, Ascrinvacumab, Aselizumab, Atezolizumab, Atidortoxumab, Atinumab, Atlizumab, Atorolimumab, Avelumab, Azintuxizumab vedotin, Bapineuzumab, Basiliximab, Bavituximab, BCD-100, Bectumomab, Begelomab, Belantamab mafodotin, Belatacept, Belimumab, Bemarituzumab, Benralizumab, Bermekimab, Bersanlimab, Bertilimumab, Besilesomab, Bevacizumab, Bevacizumab-awwb, Bezlotoxumab, Biciromab, Bimagrumab, Bimekizumab, Birtamimab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camidanlumab tesirine, Camrelizumab, Canakinumab, Cantuzumab mertansine, Cantuzumab ravtansine, Caplacizumab, Capromab pendetide, Carlumab, Carotuximab, Catumaxomab, cBR96-doxorubicin immunoconjugate, Cedelizumab, Cemiplimab, Cergutuzumab amunaleukin, Cergutuzumab amunaleukin, Certolizumab pegol, Cetrelimab, Cetuximab, Cibisatamab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, CR6261, Crizanlizumab, Crotedumab, Cusatuzumab, Dacetuzumab, Daclizumab, Dalotuzumab, Dapirolizumab pegol, Daratumumab, Dectrekumab, Demcizumab, Denileukin diftitox, Denintuzumab mafodotin, Denosumab, Depatuxizumab mafodotin, Derlotuximab biotin, Detumomab, Dezamizumab, Dinutuximab, Diridavumab, Domagrozumab, Dorlimomab aritox, Dostarlimab, Drozitumab, DS-8201, Duligotumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epitumomab cituxetan, Epoetin-alfa, Epoetin-alfa-epbx, Epratuzumab, Eptinezumab, Erenumab, Erlizumab, Ertumaxomab, Etanercept, Etanercept-szzs, Etaracizumab, Etigilimab, Etrolizumab, Evinacumab, Evolocumab, Exbivirumab, Factor VIII Fc fusion protein, Factor IX Fc fusion protein, Fanolesomab, Faralimomab, Faricimab, Farletuzumab, Fasinumab, Felvizumab, Fezakinumab, Fibatuzumab, Ficlatuzumab, Figitumumab, Filgrastim, Filgrastim-sndz, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, Ibalizumab, IBI308, Ibritumomab tiuxetan, Icrucumab, Idarucizumab, Ifabotuzumab, Igovomab, Iladatuzumab vedotin, IMAB362, Imalumab, Imaprelimab, Imciromab, Imgatuzumab, Inclacumab, Indatuximab ravtansine, Indusatumab vedotin, Inebilizumab, Infliximab, Infliximab-abda, Infliximab-dyyb, Infliximab-qbtx, Intetumumab, Inolimomab, Inotuzumab ozogamicin, Ipilimumab, Iomab-B, Iratumumab, Isatuximab, Iscalimab, Istiratumab, Itolizumab, Ixekizumab, Keliximab, Labetuzumab, Lacnotuzumab, Ladiratuzumab vedotin, Lambrolizumab, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab amadotin, Lutikizumab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pegfilgrastim-jmdb, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Racotumomab, Radretumab, Rafivirumab, Ralpancizumab, Ramucirumab, Ranevetmab, Ranibizumab, Raxibacumab, Ravagalimab, Ravulizumab, Refanezumab, Regavirumab, Relatlimab, Remtolumab, Reslizumab, Rilonacept, Rilotumumab, Rinucumab, Risankizumab, Rituximab, Rituximab-abbs, Rituximab-pvvr, Rivabazumab pegol, Rivabazumab pegol, Robatumumab, Rmab, Roledumab, Romilkimab, Romiplostim, Romosozumab, Rontalizumab, Rosmantuzumab, Rovalpituzumab tesirine, Rovalpituzumab tesirine, Rovelizumab, Rozanolixizumab, Ruplizumab, Sacituzumab govitecan, Samalizumab, Samrotamab vedotin, Sapelizumab, Sarilumab, Satralizumab (SA237), Satumomab pendetide, Secukinumab, Selicrelumab, Seribantumab, Setoxaximab, Setrusumab, Sevirumab, Sibrotuzumab, SGN-CD19A, SGN-CD33A, SHP647, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Talacotuzumab, Talizumab, Tamtuvetmab, Tanezumab, Taplitumomab paptox, Tarextumab, Tavolimab, Tefibazumab, Telimomab aritox, Telisotuzumab vedotin, Tenatumomab, Teneliximab, Teplizumab, Tepoditamab, Teprotumumab, Tesidolumab, Tetulomab, Tezepelumab, TGN1412, Tibulizumab, Ticilimumab, Tildrakizumab, Tigatuzumab, Timigutuzumab, Timolumab, Tiragotumab, Tislelizumab, Tisotumab vedotin, TNX-650, Tocilizumab, Tomuzotuximab, Toralizumab, Tosatoxumab, Tositumomab, Tovetumab, Tralokinumab, Trastuzumab, Trastuzumab-anns, Trastuzumab-dkst, Trastuzumab emtansine, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Xentuzumab, XMAB-5574, Zalutumumab, Zanolimumab, Zatuximab, Zenocutuzumab, Ziralimumab, Zolbetuximab (IMAB362, Claudiximab), Ziv-aflibercept, or Zolimomab aritox.

In other embodiments of any of the foregoing composition of matter and methods, the antibody is monoclonal. In certain embodiments, the monoclonal antibody includes but are not limited to 3F8, 8H9, Abatacept, Abagovomab, Abciximab, Abituzumab, Adalimumab-adbm, Adalimumab-atto, Adalimumab-bwwb, Abrilumab, Actoxumab, Abituzumab, Abrezekimab, Abrilumab, Actoxumab, Adalimumab, Adecatumumab, Ado-trastuzumab emtansine, Aducanumab, Afasevikumab, Afelimomab, Aflibercept, Afutuzumab, Alacizumab pegol, ALD518, Alefacept, Alemtuzumab, Alirocumab, Altumomab pentetate, Amatuximab, Anatumomab mafenatox, Andecaliximab, Anetumab ravtansine, Anifrolumab, Anrukinzumab (IMA-638) Apolizumab, Arcitumomab, Ascrinvacumab, Aselizumab, Atezolizumab, Atidortoxumab, Atinumab, Atlizumab (tocilizumab), Atorolimumab, Avelumab, Bapineuzumab, Basiliximab, Bevacizumab, Bevacizumab-awwb, BCD-100, Bectumomab, Begelomab, Belatacept, Belimumab, Bemarituzumab, Benralizumab, Bermekimab, Bersanlimab, Bertilimumab, Besilesomab, Bezlotoxumab, Biciromab, Bimagrumab, Bimekizumab, Birtamimab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camrelizumab, Canakinumab, Cantuzumab mertansine, Cantuzumab ravtansine, Caplacizumab, Capromab pendetide, Carlumab, Carotuximab, Catumaxomab, Cedelizumab, Cemiplimab, Certolizumab pegol, Cetrelimab, Cetuximab, Cibisatamab, Cirmtuzumab, Ch.14.18, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, CR6261, Crizanlizumab, Crotedumab, Cusatuzumab, Dacetuzumab, Daclizumab, Dalotuzumab, Dapirolizumab pegol, Daratumuma, Dectrekumab, Demcizumab, Denileukin diftitox, Denintuzumab mafodotin, Denosumab, Derlotuximab biotin, Detumomab, Dezamizumab, Dinutuximab, Diridavumab, Domagrozumab, Dorlimomab aritox, Dostarlimab, Drozitumab, Duligotumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emibetuzumab, Emicizumab, Enavatuzumab, Enfortumab vedotin, Enlimomab pegol, Enoblituzumab, Enokizumab, Enoticumab, Ensituximab, Epitumomab cituxetan, Epoetin-alfa, Epoetin-alfa-epbx, Epratuzumab, Eptinezumab, Erenumab, Erlizumab, Ertumaxomab, Etanercept, Etanercept-szzs, Etaracizumab, Etigilimab, Etrolizumab, Evinacumab, Evolocumab, Exbivirumab, Factor VIII Fc fusion protein, Factor IX Fc fusion protein, Fanolesomab, Faralimomab, Faricimab, Farletuzumab, Fasinumab, FBTA05, Felvizumab, Fezakinumab, Fibatuzumab, Ficlatuzumab, Figitumumab, Filgrastim, Filgrastim-sndz, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, Ibalizumab, IBI308, Ibritumomab tiuxetan, Icrucumab, Idarucizumab, Ifabotuzumab, Igovomab, IMAB362, Imalumab, Imaprelimab, Imciromab, Imgatuzumab, Inclacumab, Indatuximab ravtansine, Indusatumab vedotin, Inebilizumab, Infliximab, Infliximab-abda, Infliximab-dyyb, Infliximab-qbtx, Intetumumab, Inolimomab, Inotuzumab ozogamicin, Ipilimumab, Iratumumab, Isatuximab, Iscalimab, Istiratumab, Itolizumab, Ixekizumab, Keliximab, Labetuzumab, Lacnotuzumab, Lambrolizumab, Lampalizumab, Lanadelumab, Landogrozumab, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lutikizumab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pegfilgrastim-jmdb, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Tetulomab, Racotumomab, Radretumab, Rafivirumab, Ralpancizumab, Ramucirumab, Ranevetmab, Ranibizumab, Raxibacumab, Ravagalimab, Ravulizumab, Refanezumab, Regavirumab, Relatlimab, Remtolumab, Reslizumab, Rilonacept, Rilotumumab, Rinucumab, Risankizumab-rzaa, Rituximab, Rituximab-abbs, Rituximab-pvvr, Robatumumab, Rmab, Roledumab, Romilkimab, Romiplostim, Romosozumab, Rontalizumab, Rosmantuzumab, Rovelizumab, Rozanolixizumab, Ruplizumab, Sacituzumab govitecan, Samalizumab, Sarilumab, Satralizumab (SA237), Satumomab pendetide, Secukinumab, Selicrelumab, Seribantumab, Setoxaximab, Setrusumab, Sevirumab, Sibrotuzumab, SGN-CD19A, SGN-CD33A, SHP647, Sifalimumab, Siltuximab, Simtuzumab, Siplizumab, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Talacotuzumab, Talizumab, Tamtuvetmab, Tanezumab, Taplitumomab paptox, Tarextumab, Tavolimab, Tefibazumab, Telimomab aritox, Tenatumomab, Teneliximab, Teplizumab, Tepoditamab, Teprotumumab, Tesidolumab, Tetulomab (lilotomab), Tezepelumab, TGN1412, Tibulizumab, Ticilimumab (tremelimumab), Tildrakizumab, Tigatuzumab, Timigutuzumab, Timolumab, Tiragotumab, Tislelizumab, TNX-650, Tocilizumab (atlizumab), Tomuzotuximab, Toralizumab, Tosatoxumab, Tositumomab, Tovetumab, Tralokinumab, Trastuzumab, Trastuzumab-anns, Trastuzumab-dkst, Trastuzumab emtansine, TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Xentuzumab, XMAB-5574, Zalutumumab, Zanolimumab, Zatuximab, Zenocutuzumab, Ziralimumab, Zolbetuximab (IMAB362, Claudiximab), Ziv-aflibercept, Zolimomab aritox or the corresponding anti-drug antibody in a sample from a human patient. In preferred embodiments, the monoclonal antibody is Rituximab, Rituximab-abbs, or Rituximab-pvvr.

In some embodiments, the monoclonal antibody is a biosimilar. In other embodiments, the biosimilar includes but are not limited to Adalimumab-adbm, Adalimumab-atto, Adalimumab-bwwb, Bevacizumab-awwb, Epoetin alfa-epbx, Etanercept-szzs, Infliximab-abda, Infliximab-dyyb, Infliximab-qbtx, Filgrastim-sndz, Pegfilgrastim-jmdb, Pegfilgrastim-bmez, Risankizumab-rzaa, Rituximab-abbs, Rituximab-pvvr, Trastuzumab-anns, or Trastuzumab-dkst. In certain embodiments, the active biosimilar substance is Adalimumab, Bevacizumab, Enoxaparin sodium, Epoetin alfa, Epoetin zeta, Etanercept, Filgrastim, Follitropin alfa, Infliximab, Insulin glargine, Insulin lispro, Pegfilgrastim, Risankizumab, Rituximab, Rituximab-abbs, Rituximab-pvvr, Somatropin, Teriparatide, or Trastuzumab. In preferred embodiments, the biosimilar is Rituximab, Rituximab-abbs, or Rituximab-pvvr.

In other embodiments, the targeting moiety is an antibody from an intact polyclonal antibody, an intact monoclonal antibody, an antibody fragment, a single chain Fv (scFv) mutant, a multispecific antibody, a bispecific antibody, a chimeric antibody, a humanized antibody, a human antibody, a fusion protein comprising an antigenic determinant portion of an antibody, or other modified immunoglobulin molecules comprising antigen recognition sites.

In some embodiments, the therapeutic biologic is an immunotherapy. In other embodiments, the immunotherapy is an anti-CD20 antibody. In certain embodiments, the anti-CD20 antibody is rituximab. In certain other embodiments, the therapeutic biologic is an anti-CD20 antibody. As described herein, any antibody capable of binding the CD20 antigen may be used in the methods of the instant disclosure. Antibodies which bind the CD20 antigen include, for example: C2B8 (rituximab; RITUXAN.R™.) (U.S. Pat. No. 5,736,137, expressly incorporated herein by reference); the yttrium-[90]-labeled 2138 murine antibody designated Y2B8 (U.S. Pat. No. 5,736,137, expressly incorporated herein by reference); murine IgG2a 131 optionally labeled with 131 1 to generate the 131 1-B1 antibody (BEXXAR™.R™.) (U.S. Pat. No. 5,595,721, expressly incorporated herein by reference); murine monoclonal antibody 1F5 (Press et al. Blood 69 (2): 584-591 (1987)); chimeric 2H7 antibody (U.S. Pat. No. 5,677,180 expressly incorporated herein by reference); and monoclonal antibodies L27, G28-2, 93-1 133, B—Cl or NU—B2 available from the International Leukocyte Typing Workshop (Valentine et al., In: Leukocyte TypingIII (McMichael, Ed., p. 440, Oxford University Press (1987)).

In certain embodiments of the disclosure, the anti-CD20 antibody is rituximab. Rituximab is a genetically engineered chimeric murine/human monoclonal antibody. Rituximab is an IgG, kappa immunoglobulin containing murine light and heavy chain variable region sequences and human constant region sequences. Rituximab has a binding affinity for the CD20 antigen of approximately 8.0 nM and is commercially available, e.g., from Genentech (South San Francisco, Calif.).

In some embodiments, the therapeutic biologic is an immunotherapeutic. In other embodiments, the immunotherapeutic is a PD-1 inhibitor such as a PD-1 antibody, a PD-L1 inhibitor such as a PD-L1 antibody, a CTLA-4 inhibitor such as a CTLA-4 antibody, a CSF-1R inhibitor, an IDO inhibitor, an A1 adenosine inhibitor, an A2A adenosine inhibitor, an A2B adenosine inhibitor, an A3A adenosine inhibitor, an arginase inhibitor, or an HDAC inhibitor. In still other embodiments, the immunotherapeutic is a PD-1 inhibitor (e.g., nivolumab, pembrolizumab, pidilizumab, BMS 936559, and MPDL3280A). In some embodiments, the immunotherapy is a PD-L1 inhibitor (e.g., atezolizumab and MEDI4736). In some embodiments, the immunotherapeutic is a CTLA-4 inhibitor (e.g., ipilimumab). In certain other embodiments, the immunotherapeutic is a CSF-1R inhibitor (e.g., pexidartinib and AZD6495). In certain embodiments, the immunotherapeutic is an IDO inhibitor (e.g., norharmane, rosmarinic acid, and alpha-methyl-tryptophan). In some embodiments, the immunotherapeutic is an A1 adenosine inhibitor (e.g., 8-cyclopentyl-1,3-dimethylxanthine, 8-cyclopentyl-1,3-dipropylxanthine, 8-phenyl-1,3-dipropylxanthine, bamifylline, BG-9719, BG-9928, FK-453, FK-838, rolofylline, or N-0861). In other embodiments, the immunotherapeutic is an A2A adenosine inhibitor (e.g., ATL-4444, istradefylline, MSX-3, preladenant, SCH-58261, SCH-412,348, SCH-442,416, ST-1535, VER-6623, VER-6947, VER-7835, viadenant, or ZM-241,385). In still other embodiments, the immunotherapeutic is an A2B adenosine inhibitor (e.g., ATL-801, CVT-6883, MRS-1706, MRS-1754, OSIP-339,391, PSB-603, PSB-0788, or PSB-1115). In certain other embodiments, the immunotherapeutic is an A3A adenosine inhibitor (e.g., KF-26777, MRS-545, MRS-1191, MRS-1220, MRS-1334, MRS-1523, MRS-3777, MRE-3005-F20, MRE-3008-F20, PSB-11, OT-7999, VUF-5574, and SSR161421). In certain embodiments, the immunotherapeutic is an arginase inhibitor (e.g., an arginase antibody, (2s)-(+)-amino-5-iodoacetamidopentanoic acid, NG-hydroxy-L-arginine, (2S)-(+)-amino-6-iodoacetamidohexanoic acid, or (R)-2-amino-6-borono-2-(2-(piperidin-1-yl)ethyl)hexanoic acid. In some embodiments, the immunotherapeutic is an HDAC inhibitor (e.g., valproic acid, SAHA, or romidepsin). In other embodiments, the immunotherapeutic is a toll-like receptor activator. In still other embodiments, the immunotherapy is a RIG-I-like receptor activator. In certain other embodiments, the immunotherapeutic is a stimulator of interferon genes (STING) pathway activator. In certain embodiments, the immunotherapeutic is an Interleukin-1 receptor agonist, e.g., an IL-R1 antagonist. In some embodiments, the immunotherapeutic is a PTEN inhibitor, e.g., a bisperoxovanadium compound. In other embodiments, the immunotherapeutic is a tumor necrosis factor receptor (TNFR), e.g., TNFR-1 or TNFR-2 inhibitor. In certain embodiments, the immunotherapeutic is a Lymphocyte-activation gene 3 (LAG-3) inhibitor, e.g., GSK2831781.

In other embodiments, the therapeutic biologic is ledipasvir/sofosbuvir, insulin glargine, lenalidomide, pneumococcal 13-valent conjugate vaccine, fluticasone/salmeterol, elvitegravir/cobicistat/emtricitabine/tenofovir alafenamide, emtricitabine, rilpivirine and tenofovir alafenamide, emtricitabine/tenofovir alafenamide, grazoprevir/elbasvir, coagulation factor VIIa recombinant, epoetin alfa, Aflibercept or etanercept.

In some embodiments, the therapeutic or diagnostic agent is Abatacept, AbobotulinumtoxinA, Agalsidase beta, Albiglutide, Aldesleukin, Alglucosidase alfa, Alteplase (cathflo activase), Anakinra, Asfotase alfa, Asparaginase, Asparaginase Erwinia chrysanthemi, Becaplermin, Belatacept, Collagenase, Collagenase Clostridium histolyticum, Darbepoetin alfa, Denileukin diftitox, Dornase alfa, Dulaglutide, Ecallantide, Elosulfase alfa, Etanercept-szzs, Filgrastim, Filgrastim-sndz, Galsulfase, Glucarpidase, Idursulfase, IncobotulinumtoxinA, Interferon alfa-2b, Interferon alfa-n3, Interferon beta-1a, Interferon beta-1b, Interferon gamma-1b, Laronidase, Methoxy polyethylene glycol-epoetin beta, Metreleptin, Ocriplasmin, OnabotulinumtoxinA, Oprelvekin, Palifermin, Parathyroid hormone, Pegaspargase, Pegfilgrastim, Peginterferon alfa-2a, Peginterferon alfa-2a co-packaged with ribavirin, Peginterferon alfa-2b, Peginterferon beta-1a, Pegloticase, Rasburicase, Reteplase, Rilonacept, RimabotulinumtoxinB, Romiplostim, Sargramostim, Sebelipase alfa, Tbo-filgrastim, Tenecteplase, or Ziv-aflibercept.

In other embodiments, the diagnostic agent is tuberculin purified protein derivative, hyrotropin alpha, secretin, soluble transferrin receptor, troponin, B-type natriuretic peptide, iobenguane I 123, florbetapir F 18, perflutren, gadoterate meglumine, florbetaben F 18, flutemetamol F 18, gadoterate meglumine, isosulfan blue, regadenoson, technetium Tc 99m tilmanocept, florbetaben F 18, perflutren, regadenoson, or flutemetamol F 18.

The therapeutic or diagnostic agent in the particles may have an activity per unit of about 0.5 to about 1.0, about 0.75 to about 1.0 activity per unit, or about 0.9 to about 1.0 activity per unit. Activity is measured relative to the same therapeutic or diagnostic agent prior to particle formation. In certain embodiments, the therapeutic agent has an activity per unit of about 0.5 to about 1.0. In preferred embodiments, the therapeutic biologic has an activity per unit of about 0.5 to about 1.0. The term “activity” refers to the ratio of a functional or structural aspect of an agent, e.g., a therapeutic or diagnostic agent, at two points in time. The denominator of the ratio corresponds to a measure of the functional or structural aspect of the agent in the feed solution, immediately in advance of droplet formation. The numerator of the ratio corresponds to the same measure of a functional or structural aspect of the agent at a later point in time, e.g., immediately after particle formation.

In certain embodiments, the particles include a loading of therapeutic or diagnostic agents from about 1 to about 100 wt %, e.g., from about 50 to about 100 wt %, from about 75 to about 100 wt %, from about 90 to about 100 wt %, from about 95 to about 100 wt %, from about 99 to about 100 wt %, or from about 99.9 to about 100 wt %. At these loadings the therapeutic or diagnostic agents retain from about 0.5 to about 1.0 activity during particle formation, e.g., from about 0.75 to about 1.0 activity, from about 0.9 to about 1.0 activity, from about 0.95 to about 1.0 activity, from about 0.99 to about 1.0 activity, or from about 0.999 to about 1.0 activity. This includes the activity retained through primary desiccation (i.e., desiccation utilizing a second liquid) and, in some cases, secondary desiccation.

In some embodiments, the particles have less than about 25% internal void spaces, e.g., less than about 24, 20, 15, 10, 9, 8, 7, 6, 5, 4, 3, 2, 1, 0.5, or 0.1% internal void spaces. In certain embodiments, the particle may include less than 10% internal void spaces, less than 5% internal void spaces, less than 1% internal void spaces, less than 0.1% internal void spaces, or less than 0.01% internal void spaces. In preferred embodiments, the particle is substantially free from any internal void spaces. Suitable methods for determining internal void space can be accomplished by using Focused Ion Beam Scanning Electron Microscopy (FIB-SEM). For example, internal void space can be calculated using the following formula: internal void space=Aᵥ/Aₚ, where Aᵥ is the total area of void spaces and Aₚ is the total area of the particle.

In other embodiments, the particles may exhibit a porosity from about 0 to about 50%, e.g., from about 0 to about 10%, from about 0 to about 5%, from about 0 to about 1%, from about 0 to about 0.5%, from about 0 to about 0.1%, or from about 0 to about 0.01%. Exemplary pore size measurements include scanning electron microscopy (SEM), transmission electron microscopy (TEM), and confocal laser scanning microscopy analysis. The specific surface area of porous micro- and nanospheres may also be investigated by nitrogen adsorption/desorption analysis and a Brunauer-Emmett-Teller adsorption model. In certain embodiments where the pore sizes are sufficiently large, mercury-intrusion porosimetry may be employed.

The particles according to the disclosure are circular. Circularity can serve as an indicator of the shape of the particle. The particles described herein, can have a characteristic circularity, e.g., have a relative shape, that is substantially circular. This characteristic describes and defines the form of a particle on the basis of its circularity. The circularity is 1.0 when the particle has a completely circular structure. Particles as described herein, can have a circularity of about 0.8, 0.9, 0.95, 0.96, 0.97, 0.98, or 0.99; greater than about 0.80, greater than about 0.90, or greater than about 0.95. In some embodiments, the circularity of the particle is greater than about 0.88. In other embodiments, the circularity of the particle is greater than about 0.90. In certain embodiments, the circularity of the particle is greater than about 0.93. In preferred embodiments, the circularity of the particle is greater than about 0.97. The diameter and the circularity of the particles can be determined by the image processing of an image observed under an electron microscope or the like or a flow-type particle image analyzer. The circularity can also be determined by subjecting particles to circularity measurement and averaging the resulting values. For example, circularity (circ) can be calculated using the following formula:

$$circ = 4 * π * \frac{Area}{{Perimeter}^{2}} . \qquad Eq . 1$$

The term “perimeter”, as used herein, refers to the boundary of a closed plane figure or the sum of all borders of a two-dimensional image. As used herein, the term “area”, refers to the crossectional area of a two-dimensional image of a particle. The circularity of a particle can also be described as the ratio of the smallest diameter of the particle to its largest diameter. For a perfect circle, the ratio is 1. The percentage circularity can be calculated by multiplying the circularity by 100. The circularity can be calculated, for example, by measuring the aspect ratio using any software adapted to deal with images, for example, images obtained by microscopy, in particular, scanning electron microscopy (SEM) or transmission electron microscopy (TEM). In some embodiments, the circularity of the particles is at least about 10%, e.g., at least about 20, 30, 40, 50, 55, 60, 65, 70, 75, 80, 85, 90, 91, 92, 93, 94, 95, 96, 97, 98, 99, or about 100%. In other embodiments, the circularity of the particles is at least about 88%. In certain embodiments, the circularity of the particles is at least about 90%. In still other embodiments, the circularity of the particles is at least about 93%. In preferred embodiments, the circularity of the particles is at least about 97%.

In other embodiments, the circularity of the particle is from about 0.10 to about 1.00, e.g., from about 0.20, 0.30, 0.40, 0.50, 0.60, 0.70, 0.75, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99 to about 1.00. In certain embodiments, the circularity of the particle is from about 0.88 to about 1.00. In still other embodiments, the circularity of the particle is from about 0.90 to about 1.00. In certain other embodiments, the circularity of the particle is from about 0.93 to about 1.00. In preferred embodiments, the circularity of the particle is from about 0.97 to about 1.00. In some embodiments, methods of measuring particle circularity include image analysis of scanning electron micrographs of the particles in which the average roundness is calculated on the basis of the cross-sectional shapes of the particles projected onto the plane of the image. Such roundness factors can be extended to identify the corresponding circularity.

The particles according to the disclosure are spherical. The coefficient of sphericity of a particle is the ratio of the smallest diameter of the particle to its largest diameter. For a perfect sphere, the ratio is 1. The sphericity coefficient can be calculated by measuring the aspect ratio using any software adapted to deal with images, for example, images obtained by microscopy, in particular, scanning electron microscopy (SEM) or transmission electron microscopy (TEM). In some embodiments, the sphericity of the particles are at least about 50%, e.g., at least about 55, 60, 65, 70, 75, 80, 85, 90, 91, 92, 93, 94, 95, 96, 97, 98, 99, or about 100%. In other embodiments, the sphericity of the particle is from about 0.10 to about 1.00, e.g., from about 0.20, 0.30, 0.40, 0.50, 0.60, 0.70, 0.75, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99 to about 1.00. In preferred embodiments, the sphericity of the particle is about 1.00.

In certain embodiments, the sphericity of the particles may range from about 0.10 to about 1.00, e.g., at least about 0.20, about 0.40, about 0.60, or about 0.80 to about 1.00. In some embodiments, methods of measuring particle sphericity include image analysis of scanning electron micrographs of the particles in which the average roundness is calculated on the basis of the cross-sectional shapes of the particles projected onto the plane of the image. Sphericity (ψ) is a measure of the roundness of an object. Sphericity is the ratio of the surface area of a sphere (which has the same volume as the particle being compared) to the surface of the particle being tested. Sphericity can be calculated according to the following formula:

$$Ψ = \frac{{{π}^{\frac{1}{3}} ( 6 {V}_{p} )}^{\frac{2}{3}}}{{A}_{p}} , \qquad Eq . 2$$

where Vp is the volume of the sphere and Ap is the surface area of the sphere. The term “surface area” as used herein, refers to the external surface of a particle.

In other embodiments, the sphericity (minor axis/major axis) can be determined by using an image analyzer, or an electron microscopic photograph taken with a scanning electron microscope (SEM). For example, the average sphericity can be calculated as the average of the sphericity values calculated for randomly selected particles in the electron microscopic photograph by determining their minor axis and major axis based on visual observation.

In some embodiments of the disclosure, the drying operation may be controlled to provide particles having particular characteristics, such as particles having a substantially smooth surface. “Surface roughness”, as used herein, means a particle having numerous wrinkles or creases, e.g., being ridged or wrinkled. The term “pit”, as used herein, refers to an indentation or crevice in the particle, either an indentation or crevice in the two-dimensional image or an indentation or crevice in an object. The term “spike”, as used herein, refers to a projection pointing outward from the centroid of a particle, a projection pointing outward from the centroid of a two-dimensional image or a sharp projection pointing outward from an object.

In preferred embodiments of the disclosure, the particles as described herein, have a surface morphology that is smooth rather than ridged or wrinkled. The surface roughness of the particles may be decreased by controlling the formulation and/or process to form the particles as described herein. In certain embodiments, the drying conditions can be selected to control the particle morphology in order to enhance the smoothness of the particle's surface. In particular, the drying conditions can be selected to provide particles having a substantially smooth surface. In certain preferred embodiments, the particles have a substantially smooth surface. A person of ordinary skill in the field of this disclosure can readily assess the surface morphology of the disclosed particles using routine and standard techniques.

In other embodiments, the particle has a diameter between about 0.1 to about 1000 μm, e.g., about 0.1 to about 900, 800, 700, 600, 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, or about 0.2 μm. In certain embodiments, the particle has a diameter between about 1 to about 100 μm, e.g., about 2, 3, 4, 5, 6, 7, 8, 9, 10, 11, 12, 13, 14, 15, 16, 17, 18, 19, 20, 25, 30, 35, 40, 45, or 50 to about 100 μm. In still other embodiments, the particle has a diameter between about 4 to about 100 μm. In certain other embodiments, the particle has a diameter between about 10 to about 100 μm. In preferred embodiments, the particle has a diameter between about 20 to about 50 μm. In certain preferred embodiments, the particle is intentionally controlled in its diameter. In some embodiments, the particles have diameters from about 0.1 to about 1000 μm, e.g., about 1 to about 400 μm, about 1 to about 200 μm, about 1 to about 100 μm, about 1 to about 50 μm, about 1 to about 25 μm, about 1 to about 10 μm, about 10 to about 100 μm, about 50 to about 100 μm, about 50 to about 75 μm, or about 75 to about 100 μm. In other embodiments, the particles have diameters from about 1 to about 100 μm, e.g., from about 4 to about 100 μm, from about 10 to about 100 μm, or from about 20 to about 50 μm.

In certain embodiments, the particle has a diameter between about 0.1 to about 100 μm. In certain other embodiments, the particle has a diameter between about 0.5 to about 50 μm. In still other embodiments, the particle has a diameter between about 20 to about 50 μm. In certain preferred embodiments, the particle has a diameter between about 1 to about 40 μm. In preferred embodiments, the particle has a diameter between about 2 to about 15 μm.

In some embodiments, the particle has a surfactant content of less than about 10% by mass, e.g., less than about 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001% by mass. In other embodiments, the particle has a surfactant content of less than about 5% by mass. In certain embodiments, the particle has a surfactant content of less than about 3% by mass. In still other embodiments, the particle has a surfactant content of less than about 0.1% by mass. In certain other embodiments, the particle has a surfactant content of less than about 0.01% by mass. In some embodiments, the particle has a surfactant content of less than about 0.001% by mass. In preferred embodiments, the particle has a surfactant content of less than about 1% by mass. In certain preferred embodiments, the particle is substantially free from any surfactant content.

In other embodiments, the surfactant content of the particles is from 0 to 10 wt %, e.g., from 0 to 5 wt %, from 0 to 3 wt %, from 0 to 2 wt %, from 0 to 1 wt %, from 0 to 0.5 wt %, from 0 to 0.2 wt %, from 0 to 0.1 wt %, from 0 to 0.01 wt %, or from 0 to 0.001 wt %. Exemplary methods of measuring the surfactant content include reconstitution of the particles in an appropriate medium, e.g., deionized water, and subsequent analysis of the reconstituted solution through liquid chromatography. The chromatographic technique may include the use of a charged aerosol detector (CAD) or an evaporative light scattering detector (ELSD).

In some embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, sodium laureth sulfate, lecithin, or a combination thereof. In some embodiments, the surfactant includes, but is not limited to: (i) cationic surfactants such as; cetyltrimethylammonium chloride, hexadecyltrimethylammonium chloride, benzalkonium chloride, benzethonium chloride, dioctadecyldimethylammonium bromide; (ii) anionic surfactants such as magnesium stearate, sodium dodecyl sulfate, dioctyl sodium sulfosuccinate, sodium myreth sulfate, perfluorooctanesulfonate, alkyl ether phosphates; (iii) non-ionic surfactants such as alkylphenol ethoxylates (TritonX-100), fatty alcohol ethoxylates (octaethylene glycol monododecyl ether, cocamide diethanolamine, poloxamers, glycerolmonostearate, fatty acid esters of sorbitol (sorbitan monolaurate, Tween 80, Tween 20; and (iv) zwitterionic surfactants such as cocamidopropyl hydroxysultaine, and 3-[(3-Cholamidopropyl)dimethylammonio]-1-propanesulfonate (CHAPS). In other embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, or a combination thereof. In certain embodiments, the surfactant is polysorbate, docusate or lecithin. In preferred embodiments, the surfactant is polysorbate 20, polysorbate 60, or polysorbate 80. In certain preferred embodiments, the surfactant is polysorbate 20 or polysorbate 80. In certain other embodiments, the fatty acid ester of sorbitol is a sorbitan ester, e.g., span 20, span 40, span 60, or span 80. In still other embodiments, the surfactant is an ionic surfactant.

In other embodiments, the particles exhibit a skeletal density from about 1.00 to about 6.00 g/cm³, e.g., from about 1.00 to about 5.00 g/cm³, from about 1.00 to about 3.00 g/cm³, from about 1.00 to about 2.00 g/cm³, from about 1.00 to about 1.50 g/cm³, from about 1.30 to about 1.50 g/cm³, from about 1.32 to about 1.50 g/cm³, or from about 1.10 to about 1.40 g/cm³. In some embodiments, the particles exhibit a skeletal density from about 0.10 to about 5.00 g/cm³, e.g., from about 0.10 to about 2.50 g/cm³, from about 0.10 to about 1.40 g/cm³, from about 0.50 to about 1.40 g/cm³, or from about 1.00 to about 1.40 g/cm³. In certain embodiments, the particle has a skeletal density of about 0.09 to about 1.60 g/cm³. In still other embodiments, the particle has a skeletal density of about 1.30 to about 1.58 g/cm³. In preferred embodiments, the particle has a skeletal density of about 1.32 to about 1.50 g/cm³. Exemplary methods of skeletal density measurements include gas displacement pycnometry.

In certain embodiments, the particles have a skeletal density of about 1000 mg/mL to about 1500 mg/mL, about 1050 mg/mL to about 1500 mg/mL, about 1100 mg/mL to about 1500 mg/mL, about 1150 mg/mL to about 1500 mg/mL, about 1200 mg/mL to about 1500 mg/mL, about 1250 mg/mL to about 1500 mg/mL, about 1300 mg/mL to about 1500 mg/mL, about 1310 mg/mL to about 1500 mg/mL, about 1320 mg/mL to about 1500 mg/mL, about 1330 mg/mL to about 1500 mg/mL, about 1340 mg/mL to about 1500 mg/mL, about 1350 mg/mL to about 1500 mg/mL, about 1360 mg/mL to about 1500 mg/mL, about 1370 mg/mL to about 1500 mg/mL, about 1380 mg/mL to about 1500 mg/mL, about 1390 mg/mL to about 1500 mg/mL, about 1400 mg/mL to about 1500 mg/mL, about 1410 mg/mL to about 1500 mg/mL, about 1420 mg/mL to about 1500 mg/mL, about 1430 mg/mL to about 1500 mg/mL, about 1440 mg/mL to about 1500 mg/mL, about 1450 mg/mL to about 1500 mg/mL, about 1460 mg/mL to about 1500 mg/mL, about 1470 mg/mL to about 1500 mg/mL, about 1480 mg/mL to about 1500 mg/mL, or about 1490 mg/mL to about 1500 mg/mL.

In some embodiments, the particles can be characterized by a glass transition temperature of about 0° C. to about 250° C., e.g., of about 34° C. to about 200° C., of about 50° C. to about 200° C., of about 60° C. to about 200° C., of about 40 to about 160° C., of about 50 to about 110° C., of about 60 to about 100° C., or of about 75 to about 80° C. The term “glass transition” as used herein, refers to a thermodynamic transition of an amorphous material characterized by step changes in specific heat capacity and modulus. At temperatures above the glass transition temperature, molecular mobility is increased as are the rates of physical and chemical changes. Exemplary analytical methods for the determination of the glass transition temperature include differential scanning calorimetry and dynamic mobility analysis. In other embodiments, the particle has a glass transition temperature of about 40 to about 160° C. In still other embodiments, the particle has a glass transition temperature of about 50 to about 110° C. In certain embodiments, the particle has a glass transition temperature of about 60 to about 100° C. In preferred embodiments, the particle has a glass transition temperature of about 75 to about 80° C.

In certain embodiments, the particle has a glass transition temperature that is higher than about 160° C. In certain other embodiments, the particle has a glass transition temperature that is higher than about 90° C. In certain preferred embodiments, the particle has a glass transition temperature that is higher than about 50° C.

In some embodiments, the particle further comprises a carbohydrate, a pH adjusting agent, a salt, a chelator, a mineral, a polymer, a surfactant, a protein stabilizer, an emulsifier, an antiseptic, an amino acid, an antioxidant, a protein, an organic solvent, a paraben, a bactericide, a fungicide, a vitamin, a preservative, a nutrient media, an oligopeptide, a biologic excipient, a chemical excipient, or a combination thereof. In certain embodiments, the particle further comprises a carbohydrate, a pH adjusting agent, a salt, a surfactant, a protein stabilizer, an emulsifier, an amino acid, or a combination thereof.

In other embodiments, the carbohydrate may be from the families of monosaccharides, disaccharides, oligosaccharides, or polysaccharides. In some embodiments, the carbohydrate is dextran, trehalose, sucrose, agarose, mannitol, lactose, sorbitol, maltose, starch, alginates, xanthan, galactomanin, agar, agarose, or a combination thereof. In certain embodiments, the carbohydrate is dextran, trehalose, sucrose, agarose, mannitol, lactose, sorbitol, maltose, hydroxypropyl beta-cyclodextrin, or a combination thereof. In preferred embodiments, the carbohydrate is trehalose, cyclodextrins, hydroxypropyl beta-cyclodextrin, or a combination thereof. Cyclodextrins are available in three different forms α, β, and γ based on the number of number of glucose monomers. The number of glucose monomers in α, β, and γcyclodextrin can be 6, 7, or 8, respectively.

In some embodiments, the pH adjusting agent is acetate, citrate, glutamate, glycinate, histidine, lactate, maleate, phosphate, succinate, tartrate, bicarbonate, aluminum hydroxide, phosphoric acid, hydrochloric acid, DL-lactic/glycolic acids, phosphorylethanolamine, tromethamine, imidazole, glyclyglycine, monosodium glutamate, sodium hydroxide, potassium hydroxide, or a combination thereof. In other embodiments, the pH adjusting agent is citrate, histidine, phosphate, succinate, sodium hydroxide, potassium hydroxide, or a combination thereof. In certain embodiments, the pH adjusting agent is hydrochloric acid or citric acid.

In other embodiments, the salt is sodium chloride, calcium chloride, potassium chloride, sodium hydroxide, stannous chloride, magnesium sulfate, sodium glucoheptonate, sodium pertechnetate, guanidine hydrochloride, potassium hydroxide, or a combination thereof. In preferred embodiments, the salt is sodium chloride.

In some embodiments, the chelator is disodium edetate, ethylenediaminetetraacetic acid, pentetic acid, or a combination thereof. In other embodiments, the mineral is calcium, zinc, titanium dioxide, or a combination thereof. In certain embodiments, the polymer is propyleneglycol, glucose star polymer, silicone polymer, polydimethylsiloxane, polyethylene glycol, carboxymethylcellulose, poly(glycolic acid), poly(lactic-co-glycolic acid), polylactic acid, polycaprolactone (PCL), polyvinylpyrrolidone (PVP), ficoll, dextran, or a combination thereof.

In other embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, sodium laureth sulfate, lecithin, or a combination thereof. In some embodiments, the surfactant includes, but is not limited to: (i) cationic surfactants such as; cetyltrimethylammonium chloride, hexadecyltrimethylammonium chloride, benzalkonium chloride, benzethonium chloride, dioctadecyldimethylammonium bromide; (ii) anionic surfactants such as magnesium stearate, sodium dodecyl sulfate, dioctyl sodium sulfosuccinate, sodium myreth sulfate, perfluorooctanesulfonate, alkyl ether phosphates; (iii) non-ionic surfactants such as alkylphenol ethoxylates (TritonX-100), fatty alcohol ethoxylates (octaethylene glycol monododecyl ether, cocamide diethanolamine, poloxamers, glycerolmonostearate, fatty acid esters of sorbitol (sorbitan monolaurate, Tween 80, Tween 20; and (iv) zwitterionic surfactants such as cocamidopropyl hydroxysultaine, and 3-[(3-Cholamidopropyl)dimethylammonio]-1-propanesulfonate (CHAPS). In certain embodiments, the surfactant is polysorbate, docusate or lecithin. In preferred embodiments, the surfactant is polysorbate 20, polysorbate 60, or polysorbate 80. In certain preferred embodiments, the surfactant is polysorbate 20 or polysorbate 80. In certain other embodiments, the fatty acid ester of sorbitol is a sorbitan ester, e.g., span 20, span 40, span 60, or span 80.

In some embodiments, the protein stabilizer is acetyltryptophanate, caprylate, N-acetyltryptophan, trehalose, PEG 200, PEG 300, PEG 3350, PEG 8000, PEG 10000, PEG 20000, polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, or a combination thereof. In certain embodiments, the protein stabilizer is trehalose, PEG 200, PEG 300, PEG 3350, PEG 8000, PEG 10000, PEG 20000, polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, cyclodextrin, saccharides, hydroxypropyl beta-cyclodextrin, or a combination thereof. In preferred embodiments, the protein stabilizer is trehalose, cyclodextrin, hydroxypropyl beta-cyclodextrin, or a combination thereof. The stabilizers, used synonymously with the term “stabilizing agent”, as described herein, can be a salt, a carbohydrate, saccharides or amino acids, preferably a carbohydrate or saccharide admitted by the authorities as a suitable additive or excipient in pharmaceutical compositions. The term “excipient” refers to an additive to a preparation or formulation, which may be useful in achieving a desired modification to the characteristics of the preparation or formulation. Such modifications include, but are not limited to, physical stability, chemical stability, and therapeutic efficacy. Exemplary excipients include, but are not limited to a carbohydrate, a pH adjusting agent, a salt, a chelator, a mineral, a polymer, a surfactant, an amino acid, an oligopeptide, a biologic excipient, a chemical excipient, an antiseptic, an antioxidant, a paraben, a bactericide, a fungicide, a vitamin, a preservative, an analgesic, and/or nutrient media.

Examples of emulsifiers suitable for use in the particles include, but are not limited to, lipophilic agents having an HLB of less than 7, such as mixed fatty acid monoglycerides; mixed fatty acid diglycerides; mixtures of fatty acid mono- and diglycerides; lipophilic polyglycerol esters; glycerol esters including glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan ester including sorbitan monostearate, sorbitan sesquioleate; fatty acids and their soaps including stearic acid, palmitic acid, and oleic acid; and mixtures thereof glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan ester including sorbitan monostearate, sorbitan sesquioleate; fatty acids and their soaps including stearic acid, palmitic acid, and oleic acid; or a combination thereof. In some embodiments, the emulsifier is polysorbate 80, polysorbate 60, polysorbate 20, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof. In preferred embodiments, the fatty acid ester of sorbitol is a sorbitan ester, e.g., span 20, span 40, span 60, or span 80. In certain preferred embodiments, the emulsifier is polysorbate 80, sorbitan monooleate, or a combination thereof.

In other embodiments, the antiseptic is phenol, m-cresol, benzyl alcohol, 2-phenyloxyethanol, chlorobutanol, neomycin, benzethonium chloride, gluteraldehyde, beta-propiolactone, or a combination thereof.

In certain embodiments, the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, L-arginine, histidine, glycine, glutamine, proline, or various salts thereof (arginine hydrochloride, arginine glutamate, etc.) or a combination thereof. In preferred embodiments, the amino acid is L-arginine, histidine, proline, or a combination thereof.

In some embodiments, the antioxidant is glutathione, ascorbic acid, cysteine, N-acetyl-L-tryptophanate, tocopherol, histidine, methionine, or a combination thereof. In other embodiments, the protein is protamine, protamine sulfate, gelatin, or a combination thereof. In certain embodiments, the organic solvent is dimethyl sulfoxide, N-methyl-2-pyrrolidone, or a combination thereof. In still other embodiments, the preservative is methyl hydroxybenzoate, thimerosal, parabens, formaldehyde, castor oil, or a combination thereof. The paraben can be a parahydroxybenzoate. In some embodiments, the bactericide is benzalkonium chloride (cationic surfactants), hypochlorites, peroxides, alcohols, phenolic compounds (e.g. carbolic acid), benzyl benzoate, or a combination thereof. In preferred embodiments, the bactericide is benzyl benzoate.

In other embodiments, the fungicide is acibenzolar, 2-phenylphenol, anilazine, carvone, natamycin, potassium azide, or a combination thereof. In preferred embodiments, the fungicide is benzyl benzoate. In certain embodiments, the vitamin is thiamine, riboflavin, niacin, pantothenic acid, biotin, vitamin B6, vitamin B12, folate, niacin, ascorbic acid, calciferols, retinols, quinones, or a combination thereof. In still other embodiments, the preservative is sodium nitrate, sulfur dioxide, potassium sorbate, sodium sorbate, sodium benzoate, benzoic acid, methyl hydroxybenzoate, thimerosal, parabens, formaldehyde, castor oil, or a combination thereof. In preferred embodiments, the preservative is methyl hydroxybenzoate, thimerosal, parabens, formaldehyde, castor oil, or a combination thereof.

A number of nutrient media, preferably serum free, alone or in combination, may be used in the present disclosure, including commercially available media or other media well known in the art. Examples of such media (all without serum or having had the serum removed) include ADC-1, LPM (Bovine Serum Albumin-free), F10 (HAM), F12 (HAM), DCCM1, DCCM2, RPMI 1640, BGJ Medium (Fitton-Jackson Modification), Basal Medium Eagle (BME—with the addition of Earle's salt base), Dulbecco's Modified Eagle Medium (DMEM-without serum), Glasgow Modification Eagle Medium (GMEM), Leibovitz L-15 Medium, McCoy's 5 A Medium, Medium M199 (M199E− with Earle's salt base), Medium M199 (M199H− with Hank's salt base), Minimum Essential Medium Eagle (MEM-E− with Earle's salt base), Minimum Essential Medium Eagle (MEM-H− with Hank's salt base) and Minimum Essential Medium Eagle (MEM-NAA− with non-essential amino acids), among numerous others. In addition, serum-containing nutrient media may also be used in compositions according to the present disclosure, but the use of serum-containing media is less preferred because of the possibility that the serum may be contaminated with microbial agents and because the patient may develop immunological reactions to certain antigenic components contained in the serum.

In some embodiments, the oligopeptide is trileucine. In other embodiments, the biologic excipient are nucleic acids, oligonucleotides, antibodies or fragment thereof, amino acids, polyamino acids, peptides, proteins, cells, bacteria, gene therapeutics, genome engineering therapeutics, epigenome engineering therapeutics, hormones, nucleoproteins, glycoproteins, lipoproteins, exosomes, outer membrane vesicles, vaccines, viruses, bacteriophages, organelles, nutrient media, or a combination thereof. In certain embodiments, the chemical excipient are chemical drugs, contrast agents, dyes, magnetic particles, polymer beads, metal nanoparticles, metal microparticles, quantum dots, antioxidants, antibiotic agents, steroids, analgesics, local anesthetics, anti-inflammatory agents, parabens, anti-microbial agents, chemotherapeutic agents, vitamins, minerals, bactericides, antiseptics, or a combination thereof.

In other embodiments, the particle has less than 20% aggregation or less than 20% fragmentation of the therapeutic biologic, e.g., less than about 19, 18, 17, 16, 15, 14, 13, 12, 10, 9, 8, 7, 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1%. In some embodiments, the particle has less than 10% aggregation or less than 10% fragmentation of the therapeutic biologic, e.g., less than about 9, 8, 7, 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1%. In certain embodiments, the particle has about 3% to about 1% aggregation of the therapeutic biologic. In certain other embodiments, the particle has about 1% to about 0.5% aggregation of the therapeutic biologic. In preferred embodiments, the particle is substantially free from any aggregation of the therapeutic biologic. In still other embodiments, the particle has less than about 1% fragmentation of the therapeutic biologic. In certain preferred embodiments, the particle is substantially free from any fragmentation of the therapeutic biologic. Suitable methods for measuring aggregation and fragmentation of a biologic can be accomplished by using size-exclusion chromatography (SEC).

In some embodiments, the process of particle formation provides less than a 50% change in charge variants in the population of a diagnostic or therapeutic agent, e.g., an antibody or an antibody fragment, (e.g., less than 40, 30, 20, 10, 8, 5, 4, 3, or 1%) as compared to the therapeutic or diagnostic agent prior to particle formation. Charge variants may be acidic, basic, or neutral, and the variation may be caused post-translation modifications at terminal amino acids, such as asparagine deamidation or lysine glycation. For example, charge variants include the loss of a positive charge by the loss of a C-terminal lysine residue, covalent bonding of the amine portions of two lysine residues by reducing sugars, or the conversion of an N-terminal amine to a neutral amide by the cyclization of N-terminal glutamines. Negative charges on proteins, e.g., antibodies, can appear by the conversion of asparagine residues to aspartic acid and/or isoaspartic residues via a deamidation reaction. Exemplary methods of measuring charge variants include cation exchange chromatography (CIEX), where the variants are quantified by dividing the area under the peak corresponding to the variant, e.g., acidic, basic, or neutral population by the cumulative area contained beneath all peaks in the sample spectrum. Changes in charge variant population percentage between two samples, e.g., Sample A and Sample B, are computed as the numerical difference in the respective population variant percentages, i.e., by subtracting the specific variant, e.g., acidic, percentage of Sample B from the specific variant, e.g., acidic, percentage of Sample A, or vice versa. In certain embodiments, the analysis may be extended similarly for all variants within a population.

In certain embodiments, the particle has less than about 50% change in charge variants of the therapeutic biologic, e.g., less than about 45, 40, 35, 30, 25, 20, 15, 10, 9, 8, 7, 6, 5, 4, 3, 2, 1, 0.5, 0.1%, compared to the starting biologic prior to particle formation. In preferred embodiments, the particle is substantially free from any change in charge variants of the therapeutic biologic compared to the starting biologic prior to particle formation. Suitable methods for measuring a change in charge variants of a biologic can be accomplished by using cation exchange chromatography (CIEX).

In other embodiments, the residual moisture or solvent content of the dry component is less than about 7% by weight, e.g., less than about 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% by weight. In some embodiments, the particle has less than about 7% residual moisture by weight. In still other embodiments, the particle has less than about 5% residual moisture by weight. In certain embodiments, the particle has less than about 3% residual moisture by weight. In preferred embodiments, the particle has less than about 1% residual moisture by weight.

In some embodiments, the particle has about 1% to about 7% residual moisture by weight. In still other embodiments, the particle has about 1% to about 5% residual moisture by weight. In certain embodiments, the particle has about 1% to about 3% residual moisture by weight. In preferred embodiments, the particle is substantially free from any residual moisture by weight.

In some embodiments, the particle has greater than about 60% therapeutic biologic by weight, e.g., greater than about 65, 70, 75, 80, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% therapeutic biologic by weight. In other embodiments, the particle has greater than about 90% therapeutic biologic by weight. In certain embodiments, the particle has greater than about 95% therapeutic biologic by weight. In still other embodiments, the particle has greater than about 98% therapeutic biologic by weight. In preferred embodiments, the particle has greater than about 98% therapeutic biologic by weight. In certain preferred embodiments, the particle has greater than about 99% therapeutic biologic by weight.

The particles comprising at least one therapeutic biologic described herein, can be prepared in a number of ways, as well as any methods of forming the particles disclosed in, for example, PCT/US2021/027755, PCT/US2020/015957, PCT/US2017/063150, PCT/US2018/043774, PCT/US2019/033875, and U.S. 62/799,696, each of which is hereby incorporated by reference in its entirety. In some embodiments, methods of detecting particulate contaminants in particles or compositions comprising particles as described herein can be applied at one or more step(s) in methods of forming particles. In some aspects, methods of detecting particulate contaminants in particles as described herein are applied upon formation of the particles and prior to the particles being suspended in a liquid to form a composition (e.g., a suspension composition). In other aspects, methods of detecting particulate contaminants as described herein are applied after the particles have been combined with one or more liquids or excipients to form a composition (e.g., a suspension composition).

As used herein, the term “dispersity index” (DI) is a parameter characterizing the degree of non-uniformity of a size distribution of particles. The term “polydispersity index” (PDI) is a parameter characterizing the width of the particle size distribution within a given sample. The numerical value of PDI ranges from 0.0 (for a perfectly uniform sample with respect to the particle size) to 1.0 and greater (for a highly polydisperse sample with multiple particle size populations). As the value decreases, the particles have more narrowly distributed particle sizes, and greater homogeneity of the plurality of particles. Particle diameter may be collected using microscopy (FlowCAM, SEM) as well as laser diffraction.

In some embodiments, the polydispersity index (PDI) calculation used for Dynamic Light Scattering (DLS) measurement is: Polydispersity index from DLS=(the square of standard deviation)/(the square of mean diameter). In other embodiments, the PDI calculation can be: the statistical characteristics of particles namely number-average diameter (Dn), weight-average diameter (Dw), and polydispersity index (PDI), wherein the calculation can be accomplished using the following equations, where di represents the diameters of the microspheres, and n is the number of particles:

$${D}_{n} = \frac{\sum {d}_{i}}{n} \qquad ( 1 )$$

$${D}_{w} = \frac{\sum {( {d}_{i} )}^{4}}{\sum {( {d}_{i} )}^{3}} \qquad ( 2 )$$

$$PDI = \frac{{D}_{w}}{{D}_{n}} \qquad ( 3 )$$

In still other embodiments, polydispersity can be represented through coefficient of variation, which is calculated as: Coefficient of variation (CV=(Standard deviation×100)/mean).

In certain embodiments, the particles may include one or more agents, e.g., therapeutic biologic. In other embodiments, the particles can have diameters from about 0.1 to about 1000 μm, e.g., about 0.1 to about 90 μm, about 90 to about 230 μm, or about 0.1 to about 1 μm. In still other embodiments, the particles can have a size dispersity from about 0 to about 0.9, e.g., from about 0 to about 0.7, from about 0 to about 0.5, or from about 0 to about 0.2. Methods of measuring the particle size and distribution include imaging flow cytometry and image analysis of scanning electron micrographs of the particles in which an average spherical radius or diameter can be calculated on the basis of the cross-sectional areas of the particles projected onto the plane of the image. In certain other embodiments of the disclosure, the particle may have a diameter between about 0.1 to about 1000 μm, a skeletal density of about 1.00 to about 6.00 g/cm3, and a glass transition temperature of about 0 to about 250° C.

While each of the elements of the present disclosure is described herein, as containing multiple embodiments, it should be understood that, unless indicated otherwise, each of the embodiments of a given element of the present disclosure is capable of being used with each of the embodiments of the other elements of the present disclosure and each such use is intended to form a distinct embodiment of the present disclosure.

It will be understood by one of ordinary skill in the relevant arts that other suitable modifications and adaptations to the compositions and methods described herein are readily apparent from the description of the disclosure contained herein, in view of information known to the ordinarily skilled artisan, and may be made without departing from the scope of the disclosure or any embodiment thereof.

### Pharmaceutical Compositions

In certain embodiments, the disclosure relates to a composition comprising a plurality of particles comprising any one of the aforementioned agents suspended in a liquid (e.g., low viscosity liquid). In certain preferred embodiments, the disclosure relates to a pharmaceutical composition comprising a plurality of particles comprising any one of the aforementioned therapeutic biologics suspended in a low viscosity pharmaceutically acceptable liquid. In certain other embodiments, the disclosure relates to a composition comprising a plurality of dried particles. In some aspects, the composition comprising dried particles can be suspended in a liquid (e.g., a carrier) to form a further composition (e.g., a pharmaceutical composition), or mixed with a liquid (e.g., aqueous media) shortly before administration to a subject.

In preferred embodiments according to the disclosure as described herein, the composition comprising a plurality of particles has improved stability of the therapeutic biologic compared to an aqueous composition comprising the therapeutic biologic in monomeric form.

In other aspects, the disclosure relates to composition comprising a plurality of particles comprising an agent suspended in a liquid, wherein the particles comprise less than about 25% internal void spaces and the circularity of the particles are from about 0.10 to about 1.00. As disclosed herein, the agent may be a therapeutic or diagnostic agent. In certain embodiments, the therapeutic agent has an activity per unit of about 0.5 to about 1.0. In preferred embodiments, the therapeutic biologic has an activity per unit of about 0.5 to about 1.0.

In some embodiments, the disclosure provides a composition containing a plurality of particles that include an agent, e.g., a therapeutic or diagnostic agent, where the storage stability of the agent in the particles is improved with respect to the storage stability of the agent in the first liquid. In other embodiments, storage conditions are defined by time (e.g., more than about 2 years, more than about 1 year, more than about 6 months, more than about 3 months, more than about 1 month, or more than about 1 week) and temperature (e.g., about −80° C. to about 100° C., about −80° C. to about 60° C., about −20° C. to about 60° C., about 4 to about 60° C.), among potentially other variables. In still other embodiments, the storage time is about 3 days, about 7 days, about 30 days, about 90 days, about 180 days, about 1 year, or about 2 years. In certain other embodiments, this temperature is about −80° C., about −40° C., about −20° C., about 4° C., about 25° C., about 40° C., or about 40 to about 60° C. In certain embodiments, the storage stability of the therapeutic or diagnostic agent in the particles is improved with respect to the storage stability of a first liquid of the therapeutic or diagnostic agent.

In other embodiments, the particles have less than about 25% internal void spaces, e.g., less than about 24, 20, 15, 10, 9, 8, 7, 6, 5, 4, 3, 2, 1, 0.5, or 0.1% internal void spaces. In certain embodiments, the particles may include less than 10% internal void spaces, less than 5% internal void spaces, less than 1% internal void spaces, less than 0.1% internal void spaces, or less than 0.01% internal void spaces. In preferred embodiments, the particles are substantially free from any internal void spaces. In other embodiments, the particles may exhibit a porosity from about 0 to about 50%, e.g., from about 0 to about 10%, from about 0 to about 5%, from about 0 to about 1%, from about 0 to about 0.5%, from about 0 to about 0.1%, or from about 0 to about 0.01%. Exemplary pore size measurements include scanning electron microscopy (SEM), transmission electron microscopy (TEM), and confocal laser scanning microscopy analysis. A gallium focused ion beam (FIB) was used to cut one of the particles in half to reveal a cross-section of the particle interior. The specific surface area of porous micro- and nanospheres may also be investigated by nitrogen adsorption/desorption analysis and a Brunauer-Emmett-Teller adsorption model. In certain embodiments where the pore sizes are sufficiently large, mercury-intrusion porosimetry may be employed.

In some embodiments, the circularity of the particles are at least about 10%, e.g., at least about 20, 30, 40, 50, 55, 60, 65, 70, 75, 80, 85, 90, 91, 92, 93, 94, 95, 96, 97, 98, 99, or about 100%. In other embodiments, the circularity of the particles are at least about 88%. In certain embodiments, the circularity of the particles are at least about 90%. In still other embodiments, the circularity of the particles are at least about 93%. In preferred embodiments, the circularity of the particles are at least about 97%.

In other embodiments, the circularity of the particles are from about 0.10 to about 1.00, e.g., from about 0.20, 0.30, 0.40, 0.50, 0.60, 0.70, 0.75, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99 to about 1.00. In certain embodiments, the circularity of the particles are from about 0.88 to about 1.00. In still other embodiments, the circularity of the particles are from about 0.90 to about 1.00. In certain other embodiments, the circularity of the particles are from about 0.93 to about 1.00. In preferred embodiments, the circularity of the particles are rom about 0.97 to about 1.00.

In certain embodiments, the circularity of the particles may range from at least about 0.10 to about 1.00, e.g., at least about 0.88, about 0.90, about 0.93, or about 0.97 to about 1.00.

In some embodiments, the sphericity of the particles are at least about 50%, e.g., at least about 55, 60, 65, 70, 75, 80, 85, 90, 91, 92, 93, 94, 95, 96, 97, 98, 99, or about 100%. In other embodiments, the sphericity of the particles are from about 0.10 to about 1.00, e.g., from about 0.20, 0.30, 0.40, 0.50, 0.60, 0.70, 0.75, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99 to about 1.00. In preferred embodiments, the sphericity of the particles are about 1.00.

In certain embodiments, the sphericity of the particles may range from about 0.10 to about 1.00, e.g., at least about 0.20, about 0.40, about 0.60, or about 0.80 to about 1.00.

In preferred embodiments, the particles have a substantially smooth surface.

In some embodiments, the particles have a diameter between about 0.1 to about 1000 μm, e.g., about 0.1 to about 900, 800, 700, 600, 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, or about 0.2 μm. In certain embodiments, the particles have a diameter between about 1 to about 100 μm, e.g., about 2, 3, 4, 5, 6, 7, 8, 9, 10, 11, 12, 13, 14, 15, 16, 17, 18, 19, 20, 25, 30, 35, 40, 45, or 50 to about 100 μm. In still other embodiments, the particles have a diameter between about 4 to about 100 μm. In certain other embodiments, the particles have a diameter between about 10 to about 100 μm. In preferred embodiments, the particles have a diameter between about 20 to about 50 μm. In certain preferred embodiments, the particles are intentionally controlled in its diameter. In some embodiments, the particles have diameters from about 0.1 to about 1000 μm, e.g., about 1 to about 400 μm, about 1 to about 200 μm, about 1 to about 100 μm, about 1 to about 50 μm, about 1 to about 25 μm, about 1 to about 10 μm, about 10 to about 100 μm, about 50 to about 100 μm, about 50 to about 75 μm, or about 75 to about 100 μm. In other embodiments, the particles have diameters from about 1 to about 100 μm, e.g., from about 4 to about 100 μm, from about 10 to about 100 μm, or from about 20 to about 50 μm.

In certain embodiments, the particles have a diameter between about 0.1 to about 100 μm. In certain other embodiments, the particles have a diameter between about 0.5 to about 50 μm. In still other embodiments, the particles have a diameter between about 20 to about 50 μm. In certain preferred embodiments, the particles have a diameter between about 1 to about 40 μm. In preferred embodiments, the particles have a diameter between about 2 to about 15 μm.

In some embodiments, the particles have a surfactant content of less than about 10% by mass, e.g., less than about 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001% by mass. In other embodiments, the particles have a surfactant content of less than about 5% by mass. In certain embodiments, the particles have a surfactant content of less than about 3% by mass. In still other embodiments, the particles have a surfactant content of less than about 0.1% by mass. In certain other embodiments, the particles have a surfactant content of less than about 0.01% by mass. In some embodiments, the particles have a surfactant content of less than about 0.001% by mass. In preferred embodiments, the particles have a surfactant content of less than about 1% by mass. In certain preferred embodiments, the particles are substantially free from any surfactant content.

In other embodiments, the surfactant content of the particles are from 0 to 10 wt %, e.g., from 0 to 5 wt %, from 0 to 3 wt %, from 0 to 2 wt %, from 0 to 1 wt %, from 0 to 0.5 wt %, from 0 to 0.2 wt %, from 0 to 0.1 wt %, from 0 to 0.01 wt %, or from 0 to 0.001 wt %.

In some embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, or a combination thereof. In certain embodiments, the surfactant is polysorbate, docusate or lecithin. In preferred embodiments, the surfactant is polysorbate 20, polysorbate 60, or polysorbate 80. In certain preferred embodiments, the surfactant is polysorbate 20 or polysorbate 80. In certain other embodiments, the fatty acid ester of sorbitol is a sorbitan ester, e.g., span 20, span 40, span 60, or span 80. In still other embodiments, the surfactant is an ionic surfactant.

In other embodiments, the particles exhibit a skeletal density from about 1.00 to about 6.00 g/cm³, e.g., from about 1.00 to about 5.00 g/cm³, from about 1.00 to about 3.00 g/cm³, from about 1.00 to about 2.00 g/cm³, from about 1.00 to about 1.50 g/cm³, from about 1.30 to about 1.50 g/cm³, from about 1.32 to about 1.50 g/cm³, or from about 1.10 to about 1.40 g/cm³. In some embodiments, the particles exhibit a skeletal density from about 0.10 to about 5.00 g/cm³, e.g., from about 0.10 to about 2.50 g/cm³, from about 0.10 to about 1.40 g/cm³, from about 0.50 to about 1.40 g/cm³, or from about 1.00 to about 1.40 g/cm³. In certain embodiments, the particles have a skeletal density of about 0.09 to about 1.60 g/cm³. In still other embodiments, the particles have a skeletal density of about 1.30 to about 1.58 g/cm³. In preferred embodiments, the particles have a skeletal density of about 1.32 to about 1.50 g/cm3.

In certain embodiments, the particles have a skeletal density of about 1000 mg/mL to about 1500 mg/mL, about 1050 mg/mL to about 1500 mg/mL, about 1100 mg/mL to about 1500 mg/mL, about 1150 mg/mL to about 1500 mg/mL, about 1200 mg/mL to about 1500 mg/mL, about 1250 mg/mL to about 1500 mg/mL, about 1300 mg/mL to about 1500 mg/mL, about 1310 mg/mL to about 1500 mg/mL, about 1320 mg/mL to about 1500 mg/mL, about 1330 mg/mL to about 1500 mg/mL, about 1340 mg/mL to about 1500 mg/mL, about 1350 mg/mL to about 1500 mg/mL, about 1360 mg/mL to about 1500 mg/mL, about 1370 mg/mL to about 1500 mg/mL, about 1380 mg/mL to about 1500 mg/mL, about 1390 mg/mL to about 1500 mg/mL, about 1400 mg/mL to about 1500 mg/mL, about 1410 mg/mL to about 1500 mg/mL, about 1420 mg/mL to about 1500 mg/mL, about 1430 mg/mL to about 1500 mg/mL, about 1440 mg/mL to about 1500 mg/mL, about 1450 mg/mL to about 1500 mg/mL, about 1460 mg/mL to about 1500 mg/mL, about 1470 mg/mL to about 1500 mg/mL, about 1480 mg/mL to about 1500 mg/mL, or about 1490 mg/mL to about 1500 mg/mL.

In other embodiments, the particles can be characterized by a glass transition temperature of about 0° C. to about 250° C., e.g., of about 34° C. to about 200° C., of about 50° C. to about 200° C., of about 60° C. to about 200° C., of about 40 to about 160° C., of about 50 to about 110° C., of about 60 to about 100° C., or of about 75 to about 80° C. In other embodiments, the particles have a glass transition temperature of about 40 to about 160° C. In still other embodiments, the particles have a glass transition temperature of about 50 to about 110° C. In certain embodiments, the particles have a glass transition temperature of about 60 to about 100° C. In preferred embodiments, the particles have a glass transition temperature of about 75 to about 80° C. In still other embodiments, the particles are heated to about ±30° C., e.g., to about ±20, ±10, ±5, ±1° C., of the glass transition temperature of the particles during drying.

In certain embodiments, the particles have a glass transition temperature that is higher than about 160° C. In certain other embodiments, the particles have a glass transition temperature that is higher than about 90° C. In certain preferred embodiments, the particles have a glass transition temperature that is higher than about 50° C.

In some embodiments, the particles further comprise a carbohydrate, a pH adjusting agent, a salt, a chelator, a mineral, a polymer, a surfactant, a protein stabilizer, an emulsifier, an antiseptic, an amino acid, an antioxidant, a protein, an organic solvent, a paraben, a bactericide, a fungicide, a vitamin, a preservative, a nutrient media, an oligopeptide, a biologic excipient, a chemical excipient, or a combination thereof. In certain embodiments, the particle further comprises a carbohydrate, a pH adjusting agent, a salt, a surfactant, a protein stabilizer, an emulsifier, an amino acid, or a combination thereof.

In certain embodiments, the liquid is non-aqueous or aqueous. In other embodiments, the liquid is non-aqueous. In still other embodiments, the liquid is aqueous.

In other embodiments, the non-aqueous liquid is an organic solvent or an ionic liquid. In some embodiments, the organic solvent is benzyl benzoate, coconut oil, cottonseed oil, fish oil, grape seed oil, hazelnut oil, hydrogenated vegetable oils, olive oil, palm seed oil, peanut oil, peppermint oil, safflower oil, sesame oil, soybean oil, sunflower oil, walnut oil, acetone, ethyl acetate, ethyl lactate, dimethylacetamide, dimethyl isosorbide, dimethyl sulfoxide, glycofurol, diglyme, methyl tert-butyl ether, N-methyl pyrrolidone, perfluorodecalin, polyethylene glycol, 2-pyrrolidone, tetrahydrofurfuryl alcohol, trigylcerides, triglycerides of the fractionated plant fatty acids C8 and C10, propylene glycol diesters (e.g., propylene glycol diesters of saturated plant fatty acids C8 and C10), ethyl oleate, ethyl caprate, dibutyl adipate, fatty acid esters, hexanoic acid, octanoic acid, triacetin, diethyl glycol monoether, gamma-butyrolactone, eugenol, clove bud oil, citral, limonene, polyoxyl 40 hydrogenated castor oil, polyoxyl 35 castor oil, simple alcohols such as ethanol, octanol, hexanol, decanol, propanol, and butanol, gamma-butyrolactone, tocopherol, octa-fluoropropane, (perfluorohexyl) octane, n-acetyltryptophan, ethyl laurate, methyl caprylate, methyl caprate, methyl myristate, methyl oleate, methyl linoleate, dimethyl adipate, dibutyl suberate, diethyl sebacate, ethyl macadamiate, trimethylolpropane triisosterate, isopropyl laurate, isopropyl myristate, diethyl succinate, polysorbate esters, ethanol amine, propanoic acid, citral, anisole, anethol, benzaldehyde, linalool, caprolactone, phenol, thioglycerol, dimethylacetamide, diethylene glycol monoethyl ether, propylene carbonate, solketal, isosorbide dimethyl ether, ethyl formate, and ethyl hexyl acetate, or a combination thereof. In preferred embodiments, the organic solvent is ethyl oleate, ethyl laureate, ethyl macadamiate, ethyl caprate, diethyl succinate, diethylene glycol monoethyl ether, propylene carbonate, or a combination thereof. In certain preferred embodiments, the organic solvent is ethyl oleate. Exemplary ionic liquids of the disclosure contain (i) cations such as pyridinium, pyridazinium, pyrimidinium, pyrazinium, imidazolium, pyrazolium, thiazolium, oxazolium, triazolium, ammonium, sulfonium; and (ii) anions such as halides, sulfates, sulfonates, carbonates, phosphates, bicarbonates, nitrates, acetates, PF₆-, BF₄-, triflate, nonaflate, bis(triflyl)amide, trifluoroacetate, heptafluorobutanoate, haloaluminate, or a combination thereof. In certain embodiments, the ionic liquid comprises pyridinium, pyridazinium, pyrimidinium, pyrazinium, imidazolium, pyrazolium, thiazolium, oxazolium, triazolium, ammonium, sulfonium, halides, sulfates, sulfonates, carbonates, phosphates, bicarbonates, nitrates, acetates, PF₆-, BF₄-, triflate, nonaflate, bis(trifyl)amide, trifluoroacetate, heptafluorobutanoate, haloaluminate, or a combination thereof.

In certain embodiments, the organic solvent is acetonitrile, chlorobenzene, chloroform, cyclohexane, cumene, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, methylisobutylketone, N-methylpyrrolidone, nitromethane, pyridine, sulfolane, tetrahydrofuran, tetralin, toluene, 1,1,2-trichloroethene, xylene, acetic acid, acetone, anisole, 1-butanol, 2-butanol, butylacetate, tert-butylmethyl ether, dimethyl sulfoxide, ethanol, ethylacetate, ethyl ether, ethyl formate, formic acid, heptane, isobutylacetate, isopropylacetate, methylacetate, 3-methyl-1-butanol, methylethyl ketone, 2-methyl-1-propanol, pentane, 1-pentanol, 1-propanol, 2-propanol, propylacetate, triethylamine, 1,1-diethoxypropane, 1,1-dimethoxymethane, 2,2-dimethoxypropane, isooctane, isopropyl ether, methylisopropyl ketone, methyltetrahydrofuran, petroleum ether, trichloroacetic acid, trifluoroacetic acid, decanol, 2-ethylhexylacetate, amylacetate, or a combination thereof.

In some embodiments, the aqueous liquid is water, 0.9% saline, lactated Ringer's solution, buffers, dextrose 5%, or a combination thereof. In preferred embodiments, the aqueous liquid is water. Exemplary buffers of the disclosure may include acetate buffer, histidine buffer, succinate buffer, HEPES buffer, tris buffer, carbonate buffer, citrate buffer, phosphate buffer, phosphate-buffered saline, glycine buffer, barbital buffer, cacodylate buffer, ammonium formate buffer, urea solution, or a combination thereof.

The phrase “pharmaceutically acceptable” is employed herein, to refer to those therapeutic biologics, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The term “pharmaceutically acceptable” can refer to particles and compositions comprising a plurality of particles that do not produce an adverse, allergic, or other untoward reaction when administered to a mammal, such as a human, as appropriate. The preparation of a pharmaceutical composition comprising an antibody or additional active ingredient will be known to those of skill in the art in light of the present disclosure. Moreover, for mammal (e.g., human) administration, it will be understood that preparations should meet sterility, pyrogenicity, general safety, and purity standards as required by FDA Office of Biological Standards.

The phrase “pharmaceutically acceptable liquid” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters. In certain preferred embodiments, the plurality of particles is suspended in a pharmaceutically acceptable liquid. In preferred embodiments, the liquid is a pharmaceutically acceptable liquid.

A pharmaceutical composition (formulation) as disclosed herein, can be administered to a subject by any of a number of routes of administration including, for example, parenterally (including intramuscularly, intravenously, subcutaneously or intrathecally as, for example, a sterile solution or suspension); intraperitoneally; or subcutaneously. In certain embodiments, a composition may be simply suspended in a non-aqueous liquid carrier. Details of appropriate routes of administration and compositions suitable for same can be found in, for example, U.S. Pat. Nos. 6,110,973; 5,763,493; 5,731,000; 5,541,231; 5,427,798; 5,358,970 and 4,172,896, as well as in patents cited therein. The term “suspension formulation” refers to a liquid formulation including solid particles disposed within a carrier liquid in which they are not soluble on an appropriate timescale. The particles may settle over time, i.e., the physical stability of the suspension is not indefinite, but may be re-suspended using a form of agitation or excitation.

A “therapeutic amount” refers to an amount of a therapeutic or diagnostic agent required to produce the desired effect. As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

In certain embodiments, the liquid further comprises a carbohydrate, a pH adjusting agent, a salt, a chelator, a mineral, a polymer, a surfactant, a protein stabilizer, an emulsifier, an antiseptic, an amino acid, an antioxidant, a protein, an organic solvent, a paraben, a bactericide, a fungicide, a vitamin, a preservative, a nutrient media, analgesic, or a combination thereof. In preferred embodiments, the liquid further comprises a carbohydrate, a pH adjusting agent, a salt, a surfactant, a protein stabilizer, an emulsifier, an amino acid, or a combination thereof. In certain preferred embodiments, the aqueous liquid further comprises a carbohydrate, a pH adjusting agent, a salt, a surfactant, a protein stabilizer, an emulsifier, an amino acid, or a combination thereof.

In other embodiments, the carbohydrate may be from the families of monosaccharides, disaccharides, oligosaccharides, or polysaccharides. In some embodiments, the carbohydrate is dextran, trehalose, sucrose, agarose, mannitol, lactose, sorbitol, maltose, starch, alginates, xanthan, galactomanin, agar, agarose, or a combination thereof. In certain embodiments, the carbohydrate is dextran, trehalose, sucrose, agarose, mannitol, lactose, sorbitol, maltose, hydroxypropyl beta-cyclodextrin, or a combination thereof. In preferred embodiments, the carbohydrate is trehalose, cyclodextrins, hydroxypropyl beta-cyclodextrin, or a combination thereof. Cyclodextrins are available in three different forms α, β, and γ based on the number of number of glucose monomers. The number of glucose monomers in α, β, and γcyclodextrin can be 6, 7, or 8, respectively.

In some embodiments, the pH adjusting agent is acetate, citrate, glutamate, glycinate, histidine, lactate, maleate, phosphate, succinate, tartrate, bicarbonate, aluminum hydroxide, phosphoric acid, hydrochloric acid, DL-lactic/glycolic acids, phosphorylethanolamine, tromethamine, imidazole, glyclyglycine, monosodium glutamate, sodium hydroxide, potassium hydroxide, or a combination thereof. In other embodiments, the pH adjusting agent is citrate, histidine, phosphate, succinate, sodium hydroxide, potassium hydroxide, or a combination thereof. In certain embodiments, the pH adjusting agent is hydrochloric acid or citric acid.

In other embodiments, the salt is sodium chloride, calcium chloride, potassium chloride, sodium hydroxide, stannous chloride, magnesium sulfate, sodium glucoheptonate, sodium pertechnetate, guanidine hydrochloride, potassium hydroxide, or a combination thereof. In preferred embodiments, the salt is sodium chloride.

In some embodiments, the chelator is disodium edetate, ethylenediaminetetraacetic acid, pentetic acid, or a combination thereof. In other embodiments, the mineral is calcium, zinc, titanium dioxide, or a combination thereof. In certain embodiments, the polymer is propyleneglycol, glucose star polymer, silicone polymer, polydimethylsiloxane, polyethylene glycol, carboxymethylcellulose, poly(glycolic acid), poly(lactic-co-glycolic acid), polylactic acid, polycaprolactone (PCL), polyvinylpyrrolidone (PVP), ficoll, dextran, or a combination thereof.

In other embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, sodium laureth sulfate, lecithin, or a combination thereof. In some embodiments, the surfactant includes, but is not limited to: (i) cationic surfactants such as; cetyltrimethylammonium chloride, hexadecyltrimethylammonium chloride, benzalkonium chloride, benzethonium chloride, dioctadecyldimethylammonium bromide; (ii) anionic surfactants such as magnesium stearate, sodium dodecyl sulfate, dioctyl sodium sulfosuccinate, sodium myreth sulfate, perfluorooctanesulfonate, alkyl ether phosphates; (iii) non-ionic surfactants such as alkylphenol ethoxylates (TritonX-100), fatty alcohol ethoxylates (octaethylene glycol monododecyl ether, cocamide diethanolamine, poloxamers, glycerolmonostearate, fatty acid esters of sorbitol (sorbitan monolaurate, Tween 80, Tween 20; and (iv) zwitterionic surfactants such as cocamidopropyl hydroxysultaine, and 3-[(3-Cholamidopropyl)dimethylammonio]-1-propanesulfonate (CHAPS). In certain embodiments, the surfactant is polysorbate, docusate or lecithin. In preferred embodiments, the surfactant is polysorbate 20 or polysorbate 80.

In some embodiments, the protein stabilizer is acetyltryptophanate, caprylate, N-acetyltryptophan, trehalose, PEG 200, PEG 300, PEG 3350, PEG 8000, PEG 10000, PEG 20000, polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, or a combination thereof. In certain embodiments, the protein stabilizer is trehalose, PEG 200, PEG 300, PEG 3350, PEG 8000, PEG 10000, PEG 20000, polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, cyclodextrin, saccharides, or a combination thereof. In preferred embodiments, the protein stabilizer is trehalose, PEG 200, PEG 300, PEG 3350, PEG 8000, PEG 10000, PEG 20000, cyclodextrin, hydroxypropyl beta-cyclodextrin, or a combination thereof. The stabilizers, used synonymously with the term “stabilizing agent”, as described herein, can be a salt, a carbohydrate, saccharides or amino acids, preferably a carbohydrate or saccharide admitted by the authorities as a suitable additive or excipient in pharmaceutical compositions. The term “stabilizer” refers to an excipient or a mixture of excipients which stabilizes the physical and/or chemical properties of agents, e.g., therapeutic or diagnostic agents. In some embodiments, stabilizers prevent, e.g., degradation of the therapeutic or diagnostic agents during droplet formation, desiccation, and/or storage of the particulate matter. Exemplary stabilizers include, but are not limited to, sugars, salts, hydrophobic salts, detergents, reducing agents, cyclodextrins, polyols, carboxylic acids, and amino acids. A “stable” formulation as described herein, refers to a formulation in which the therapeutic or diagnostic agent retains an acceptable portion of its essential physical, chemical, or biological properties over an acceptable period of time. In the case of proteins and peptides, e.g., exemplary methods of assessing stability are reviewed in (i) Peptide and Protein Drug Delivery, 247-301, Vincent Lee Ed., Marcel Dekker, Inc., New York, NY, 1991, and (ii) Jones, A., Adv. Drug Delivery Rev. 10:29-90 (1993). In certain embodiments, chemical stability of a protein is assessed by measuring the size distribution of the sample at several stages. These include, e.g., before particle formation (assessment of the feed solution), immediately after particle formation, and again after a period of storage, where storage takes place either within or in the absence of a suspension formulation carrier medium. In certain other embodiments, the size distribution is assessed by size exclusion chromatography (SEC-HPLC).

Examples of emulsifiers suitable for use in the liquid include, but are not limited to, lipophilic agents having an HLB of less than 7, such as mixed fatty acid monoglycerides; mixed fatty acid diglycerides; mixtures of fatty acid mono- and diglycerides; lipophilic polyglycerol esters; glycerol esters including glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan ester including sorbitan monostearate, sorbitan sesquioleate; fatty acids and their soaps including stearic acid, palmitic acid, and oleic acid; and mixtures thereof glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan ester including sorbitan monostearate, sorbitan sesquioleate; fatty acids and their soaps including stearic acid, palmitic acid, and oleic acid; or a combination thereof. In some embodiments, the emulsifier is polysorbate 80, polysorbate 20, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof. In preferred embodiments, the emulsifier is polysorbate 80, sorbitan monooleate, or a combination thereof.

In other embodiments, the antiseptic is phenol, m-cresol, benzyl alcohol, 2-phenyloxyethanol, chlorobutanol, neomycin, benzethonium chloride, gluteraldehyde, beta-propiolactone, or a combination thereof.

In certain embodiments, the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, L-arginine, histidine, glycine, glutamine, proline, or a combination thereof. In preferred embodiments, the amino acid is L-arginine, histidine, proline, or a combination thereof.

In some embodiments, the antioxidant is glutathione, ascorbic acid, cysteine, N-acetyl-L-tryptophanate, tocopherol, histidine, methionineor tocopherol, or a combination thereof. In other embodiments, the protein is protamine, protamine sulfate, gelatin, or a combination thereof. In certain embodiments, the organic solvent is dimethyl sulfoxide, N-methyl-2-pyrrolidone, or a combination thereof. In still other embodiments, the preservative is methyl hydroxybenzoate, thimerosal, parabens, formaldehyde, castor oil, or a combination thereof. In certain other embodiments, the preservative is sodium nitrate, sulfur dioxide, potassium sorbate, sodium sorbate, sodium benzoate, benzoic acid, methyl hydroxybenzoate, thimerosal, parabens, formaldehyde, castor oil, or a combination thereof. The paraben can be a parahydroxybenzoate. In some embodiments, the bactericide is benzalkonium chloride (cationic surfactants), hypochlorites, peroxides, alcohols, phenolic compounds (e.g. carbolic acid), or a combination thereof.

In other embodiments, the fungicide is acibenzolar, 2-phenylphenol, anilazine, carvone, natamycin, potassium azide, or a combination thereof. In certain embodiments, the vitamin is thiamine, riboflavin, niacin, pantothenic acid, biotin, vitamin B6, vitamin B12, folate, niacin, ascorbic acid, calciferols, retinols, quinones, or a combination thereof.

A number of nutrient media, preferably serum free, alone or in combination, may be used in the present disclosure, including commercially available media or other media well known in the art. Examples of such media (all without serum or having had the serum removed) include ADC-1, LPM (Bovine Serum Albumin-free), F10 (HAM), F12 (HAM), DCCM1, DCCM2, RPMI 1640, BGJ Medium (Fitton-Jackson Modification), Basal Medium Eagle (BME—with the addition of Earle's salt base), Dulbecco's Modified Eagle Medium (DMEM-without serum), Glasgow Modification Eagle Medium (GMEM), Leibovitz L-15 Medium, McCoy's 5 A Medium, Medium M199 (M199E− with Earle's salt base), Medium M199 (M199H− with Hank's salt base), Minimum Essential Medium Eagle (MEM-E− with Earle's salt base), Minimum Essential Medium Eagle (MEM-H− with Hank's salt base) and Minimum Essential Medium Eagle (MEM-NAA− with non-essential amino acids), among numerous others. In addition, serum-containing nutrient media may also be used in compositions according to the present disclosure, but the use of serum-containing media is less preferred because of the possibility that the serum may be contaminated with microbial agents and because the patient may develop immunological reactions to certain antigenic components contained in the serum.

In some embodiments, the analgesic is paracetamol, histamine receptor antagonist (e.g., an H1 or an H2 blocker), NSAIDs, COX-2 inhibitors, Celecoxib, Rofecoxib, Valdecoxib, Parecoxib, Lumiracoxib, Etoricoxib, Firocoxib, acetaminophen, opiates, Dextropropoxyphene, Codeine, Tramadol, Anileridine, Pethidine, Hydrocodone, Morphine, Oxycodone, Methadone, Diacetylmorphine, Hydromorphone, Oxymorphone, Levorphanol, Buprenorphine, Fentanyl, Sufentanyl, Etorphine, Carfentanil, dihydromorphine, dihydrocodeine, Thebaine, Papaverine, diproqualone, Flupirtine, Tricyclic antidepressants, Acetaminophen or lidocaine, or a combination thereof. In certain embodiments, the analgesic is acetaminophen or lidocaine.

In certain embodiments, the liquid further comprises at least one pharmaceutically acceptable additive, diluent, excipient, carrier, or a combination thereof. In certain other embodiments, the liquid further comprises a second agent. In other embodiments, the liquid further comprises a second diagnostic or therapeutic agent.

In some embodiments, the particles have less than 20% aggregation or less than 20% fragmentation of the therapeutic biologic, e.g., less than about 19, 18, 17, 16, 15, 14, 13, 12, 10, 9, 8, 7, 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1%. In other embodiments, the particles have less than 10% aggregation or less than 10% fragmentation of the therapeutic biologic, e.g., less than about 9, 8, 7, 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1%. In certain embodiments, the particles have about 3% to about 1% aggregation of the therapeutic biologic. In certain other embodiments, the particles have about 1% to about 0.5% aggregation of the therapeutic biologic. In preferred embodiments, the particles are substantially free from any aggregation of the therapeutic biologic. In still other embodiments, the particles have less than about 1% fragmentation of the therapeutic biologic. In certain preferred embodiments, the particles are substantially free from any fragmentation of the therapeutic biologic.

In certain embodiments, the methods described herein, may further include suspending the particles in a pharmaceutically acceptable medium, e.g., reconstitution of the dried particles. In some embodiments, the dissolution or reconstitution of the particles provides less than about 10% increase in aggregates of the diagnostic or therapeutic agent, e.g., a protein, (e.g., less than about 8%, less than about 5%, less than about 4%, less than about 3%, less than about 1%, less than about 0.5%, or less than about 0.1%) as compared to the therapeutic or diagnostic agent in the first liquid prior to processing. Exemplary methods of measuring aggregates include size exclusion high-performance liquid chromatography (SEC-HPLC), where the aggregate population is quantified by dividing the area under the peak corresponding to the aggregate population by the cumulative area contained beneath all peaks in the sample spectrum. Changes in aggregate percentage between two samples, e.g., Sample A and Sample B, are computed as the numerical difference in the respective aggregate percentages, i.e., by subtracting the aggregate percentage of Sample B from the aggregate percentage of Sample A, or vice versa. In certain other embodiments, the dissolution or reconstitution of the particles provides less than about 10% increase in fragments of the diagnostic or therapeutic agent, e.g., a protein, (e.g., less than about 8%, less than about 5%, less than about 4%, less than about 3%, less than about 1%, less than about 0.5%, or less than about 0.1%) as compared to the therapeutic or diagnostic agent in the first liquid prior to processing. Exemplary methods of measuring fragments include size exclusion high-performance liquid chromatography (SEC-HPLC), where the fragment population is quantified by dividing the area under the peak corresponding to the fragment population by the cumulative area contained beneath all peaks in the sample spectrum. Changes in fragment percentage between two samples, e.g., Sample A and Sample B, are computed as the numerical difference in the respective fragment percentages, i.e., by subtracting the fragment percentage of Sample B from the fragment percentage of Sample A, or vice versa.

In other embodiments, the process of particle formation provides less than a 50% change in charge variants in the population of a diagnostic or therapeutic agent, e.g., an antibody or an antibody fragment, (e.g., less than 40, 30, 20, 10, 8, 5, 4, 3, or 1%) as compared to the therapeutic or diagnostic agent prior to particle formation. In certain embodiments, the particles have less than about 50% change in charge variants of the therapeutic biologic, e.g., less than about 45, 40, 35, 30, 25, 20, 15, 10, 9, 8, 7, 6, 5, 4, 3, 2, 1, 0.5, 0.1%, compared to the starting biologic prior to particle formation. In preferred embodiments, the particles are substantially free from any change in charge variants of the therapeutic biologic compared to the starting biologic prior to particle formation.

In some embodiments, the residual moisture or solvent content of the dry component is less than about 7% by weight, e.g., less than about 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% by weight. In other embodiments, the particles have less than about 7% residual moisture by weight. In still other embodiments, the particles have less than about 5% residual moisture by weight. In certain embodiments, the particles have less than about 3% residual moisture by weight. In preferred embodiments, the particles have than about 1% residual moisture by weight.

In other embodiments, the particles have about 1% to about 7% residual moisture by weight. In still other embodiments, the particles have about 1% to about 5% residual moisture by weight. In certain embodiments, the particles have about 1% to about 3% residual moisture by weight. In preferred embodiments, the particles are substantially free from any residual moisture by weight.

In some embodiments, the particles have greater than about 60% therapeutic biologic by weight, e.g., greater than about 65, 70, 75, 80, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% therapeutic biologic by weight. In other embodiments, the particles have greater than about 90% therapeutic biologic by weight. In certain embodiments, the particles have greater than about 95% therapeutic biologic by weight. In still other embodiments, the particles have greater than about 98% therapeutic biologic by weight. In preferred embodiments, the particles have greater than about 98% therapeutic biologic by weight. In certain preferred embodiments, the particles have greater than about 99% therapeutic biologic by weight.

The concentration of the therapeutic biologic in the composition is typically of about 20 mg/mL to about 650 mg/mL, e.g., about 20, 30, 50, 75, 100, 125, 150, 175, 200, 225, 250, 275, 300, 325, 350, 375, 400, 425, 450, 475, 500, 525, 550, 575, 600, 625 mg/mL to about 650 mg/mL. The therapeutic biologic in the composition may have about 0.5 to about 1.0 activity per unit, about 0.75 to about 1.0 activity per unit, or about 0.9 to about 1.0 activity per unit. Activity is measured relative to the same therapeutic biologic prior to particle formation. In preferred embodiments, the therapeutic biologic has an activity per unit of about 0.5 to about 1.0.

In some embodiments, the compositions described herein, use a concentration of the therapeutic biologic in the composition of about 20 mg/mL to about 650 mg/mL, e.g., about 20, 30, 50, 75, 100, 125, 150, 175, 200, 225, 250, 275, 300, 325, 350, 375, 400, 425, 450, 475, 500, 525, 550, 575, 600, 625 mg/mL to about 650 mg/mL; about 20 mg/mL to about 625 mg/mL, e.g., about 20, 30, 50, 75, 100, 125, 150, 175, 200, 225, 250, 275, 300, 325, 350, 375, 400, 425, 450, 475, 500, 525, 550, 575, 600 mg/mL to about 625 mg/mL; about 20 mg/mL to about 600 mg/mL, e.g., about 20, 30, 50, 75, 100, 125, 150, 175, 200, 225, 250, 275, 300, 325, 350, 375, 400, 425, 450, 475, 500, 525, 550, 575 mg/mL to about 600 mg/mL; about 20 mg/mL to about 575 mg/mL, e.g., about 20, 30, 50, 75, 100, 125, 150, 175, 200, 225, 250, 275, 300, 325, 350, 375, 400, 425, 450, 475, 500, 525, 550 mg/mL to about 575 mg/mL; about 20 mg/mL to about 550 mg/mL, e.g., about 20, 30, 50, 75, 100, 125, 150, 175, 200, 225, 250, 275, 300, 325, 350, 375, 400, 425, 450, 475, 500, 525 mg/mL to about 550 mg/mL; about 20 mg/mL to about 525 mg/mL, e.g., about 20, 30, 50, 75, 100, 125, 150, 175, 200, 225, 250, 275, 300, 325, 350, 375, 400, 425, 450, 475, 500 mg/mL to about 525 mg/mL; about 20 mg/mL to about 500 mg/mL, e.g., about 20, 30, 50, 75, 100, 125, 150, 175, 200, 225, 250, 275, 300, 325, 350, 375, 400, 425, 450, 475 mg/mL to about 500 mg/mL; about 20 mg/mL to about 475 mg/mL, e.g., about 20, 30, 50, 75, 100, 125, 150, 175, 200, 225, 250, 275, 300, 325, 350, 375, 400, 425, 450 mg/mL to about 475 mg/mL; about 20 mg/mL to about 450 mg/mL, e.g., about 20, 30, 50, 75, 100, 125, 150, 175, 200, 225, 250, 275, 300, 325, 350, 375, 400, 425 mg/mL to about 450 mg/mL; about 20 mg/mL to about 425 mg/mL, e.g., about 20, 30, 50, 75, 100, 125, 150, 175, 200, 225, 250, 275, 300, 325, 350, 375, 400 mg/mL to about 425 mg/mL; about 20 mg/mL to about 400 mg/mL, e.g., about 20, 30, 50, 75, 100, 125, 150, 175, 200, 225, 250, 275, 300, 325, 350, 375 mg/mL to about 400 mg/mL; about 20 mg/mL to about 375 mg/mL, e.g., about 20, 30, 50, 75, 100, 125, 150, 175, 200, 225, 250, 275, 300, 325, 350 mg/mL to about 375 mg/mL; about 20 mg/mL to about 350 mg/mL, e.g., about 20, 30, 50, 75, 100, 125, 150, 175, 200, 225, 250, 275, 300, 325 mg/mL to about 350 mg/mL; about 20 mg/mL to about 325 mg/mL, e.g., about 20, 30, 50, 75, 100, 125, 150, 175, 200, 225, 250, 275, 300 mg/mL to about 325 mg/mL; or about 20 mg/mL to about 300 mg/mL, e.g., about 20, 30, 50, 75, 100, 125, 150, 175, 200, 225, 250, 275 mg/mL to about 300 mg/mL. In other embodiments, the concentration of the therapeutic biologic in the composition is about 30 mg/mL to about 500 mg/mL. In certain embodiments, the concentration of the therapeutic biologic in the composition is about 100 mg/mL to about 500 mg/mL. In still other embodiments, the concentration of the therapeutic biologic in the composition is about 200 mg/mL to about 400 mg/mL. In preferred embodiments, the concentration of the therapeutic biologic in the composition is about 300 mg/mL to about 400 mg/mL. In certain preferred embodiments, the concentration of the therapeutic biologic in the composition is about 350 mg/mL to about 400 mg/mL.

In other embodiments, the composition has a viscosity of less than about 200 mPa·s, less than about 150 mPa·s, less than about 125 mPa·s, less than about 100 mPa·s, less than about 75 mPa·s, less than about 75 mPa·s, less than about 70 mPa·s, less than about 65 mPa·s, less than about 60 mPa·s, less than about 55 mPa·s, less than about 50 mPa·s, less than about 45 mPa·s, less than about 40 mPa·s, less than about 35 mPa·s, less than about 30 mPa·s, less than about 25 mPa·s, less than about 20 mPa·s, less than about 19 mPa·s, less than about 18 mPa·s, less than about 17 mPa·s, less than about 16 mPa·s, less than about 15 mPa·s, less than about 14 mPa·s, less than about 13 mPa·s, less than about 12 mPa·s, less than about 11 mPa·s, less than about 10 mPa·s, less than about 9.5 mPa·s, less than about 9 mPa·s, less than about 8.5 mPa·s, less than about 8 mPa·s, less than about 7.5 mPa·s, less than about 7 mPa·s, less than about 6.5 mPa·s, less than about 6 mPa·s, less than about 5.5 mPa·s, less than about 5 mPa·s, less than about 4.5 mPa·s, less than about 4 mPa·s, less than about 3.5 mPa·s, less than about 3 mPa·s, less than about 2.5 mPa·s, less than about 2 mPa·s, less than about 1.5 mPa·s, less than about 1 mPa·s, less than about 0.5 mPa·s, less than about 0.1 mPa·s, less than about 0.05 mPa·s, or less than about 0.01 mPa·s (one millipascal-second). In other embodiments, the composition has a viscosity of about 0.01 mPa·s to about 10,000 mPa·s, e.g., from about 0.01 mPa·s to about 1,000 mPa·s, from about 0.01 mPa·s to about 100 mPa·s, from about 0.01 mPa·s to about 50 mPa·s, from about 0.01 mPa·s to about 25 mPa·s, from about 0.01 mPa·s to about 10 mPa·s, from about 0.01 mPa·s to about 5 mPa·s, or from about 0.01 mPa·s to about 1 mPa·s. In certain embodiments, the viscosity of the composition can range from about 0.27 mPa·s to about 200 mPa·s, e.g., about 0.27 mPa·s to about 50 mPa·s, about 1 mPa·s to about 30 mPa·s, or about 20 mPa·s to about 50 mPa·s. In still other embodiments, the viscosity of the composition ranges from about 0.27 mPa·s to about 200 mPa·s, e.g., about 0.27 mPa·s to about 100 mPa·s, about 0.27 mPa·s to about 50 mPa·s, about 0.27 mPa·s to about 30 mPa·s, about 1 mPa·s to about 20 mPa·s, or about 1 mPa·s to about 15 mPa·s. The term “viscosity” is used to describe the property of a fluid acting to resist shearing flow. For the purposes of the present disclosure, viscosity can be determined using a rheometer, e.g., AR-G2 Rheometer (TA Instruments, USA), fitted with a cone and plate (2°/40 mm) at 25° C. at a specified shear rate. In certain embodiments, the viscosity is measured at a shear rate in the Newtonian regime. The term “Newtonian regime” means a range of shear rates which are linearly proportional or nearly linearly proportional to the local strain rate at every point. In some embodiments, the viscosity is measured at a shear rate of about 100 s⁻¹ or greater, e.g., at about 1000 s⁻¹ or greater than about 1000 s⁻¹. The composition may include from about 5 to about 90% particles by volume, e.g., e.g., about 20 to about 90%, about 40 to about 80%, about 50 to about 60%, or about 70 to about 90%. The composition may have a concentration of the therapeutic biologic from about 0.0001 to about 1000 mg/mL, e.g., from about 100 to about 900, about 150 to about 800, or about 200 to about 700 mg/mL. Methods of controlling viscosity include temperature regulation and viscosity modifying additives. Mixtures of liquids may also be used to control viscosity. The units “mPa·s” and “cP” are used herein, interchangeably in the broadest sense.

In some embodiments, the composition has a viscosity of less than about 50 mPa·s. In other embodiments, the composition has a viscosity of less than about 30 mPa·s. In still other embodiments, the composition has a viscosity of less than about 20 mPa·s. In certain other embodiments, the composition has a viscosity of less than about 10 mPa·s. In certain embodiments, the composition has a viscosity of less than about 5 mPa·s. In preferred embodiments, the composition has a viscosity of less than about 3 mPa·s. In certain preferred embodiments, the composition has a viscosity of less than about 2.5 mPa·s.

In other embodiments of the composition described herein, the plurality of particles has a polydispersity index from about 0.002 to about 1.000, e.g., from about 0.003, 0.004, 0.005, 0.006, 0.007, 0.008, 0.009, 0.010, 0.020, 0.030, 0.040, 0.050, 0.060, 0.070, 0.080, 0.090, 0.100, 0.200, 0.300, 0.400, 0.500, 0.600, 0.700, 0.800, 0.900 to about 1.000. In certain embodiments, the plurality of particles has a polydispersity index from about 0.002 to about 0.900.

In certain embodiments of the disclosure described herein, high concentrations of the therapeutic biologic in the particles and high concentrations of particles in the liquid are possible. In some embodiments, the latter may be achieved by mixing particles of various sizes.

In preferred embodiments according to the disclosure as described herein, the composition comprising a plurality of particles has improved stability of the therapeutic biologic compared to an aqueous composition comprising the therapeutic biologic in monomeric form.

In other embodiments, the particles of the disclosure can be suspended in an aqueous liquid carrier, non-aqueous liquid carrier, e.g., an organic liquid, an ionic liquid carrier, a gel carrier, or a combination thereof to form a composition (e.g., a suspension composition). The medium for the composition may further include, e.g., a carbohydrate, a pH adjusting agent, a salt, a chelator, a mineral, a polymer, a surfactant, an amino acid, an oligopeptide, a biologic excipient, a chemical excipient, an antiseptic, an antioxidant, a paraben, a bactericide, a fungicide, a vitamin, a preservative, an analgesic, and/or nutrient media. In some embodiments, each of the other components can be, independently, at about 0.0001 to about 99% (w/v) of the medium, e.g., at about 0.0001 to about 90% (w/v), at about 0.0001 to about 50% (w/v), at about 0.0001 to about 10% (w/v), at about 0.0001 to about 1% (w/v), or at about 0.0001 to about 0.1% (w/v). In certain embodiments, the disclosure provides a plurality of particles described herein, suspended in a liquid. The liquid may be an organic solvent, ionic liquid, an aqueous liquid, or a combination thereof. The liquid may further include a second diagnostic or therapeutic agent.

In some embodiments, particulate contaminants (e.g., insoluble particulate matter) with characteristic sizes greater than or equal to about 100 μm persist upon dissolution in an aqueous liquid. In some embodiments of the disclosure described herein, the composition is substantially free of particulate contaminants. In some embodiments, the aqueous liquid is water, aqueous buffer or a physiologically relevant aqueous liquid. In other embodiments, particulate contaminants which is visible to the naked eye under prescribed lighting conditions persist upon reconstitution of the particles of the disclosure into a liquid pharmaceutical composition. Particulate contaminants may be present in quantities from about 0 to about 1 per about 1 mL, e.g., from about 0 to about 0.01 per about 1 mL, from about 0 to about 0.001 per about 1 mL, or about 0 to about 0.0001 per about 1 mL. Example methods of measuring particulate contaminants include analysis of the therapeutic or diagnostic agent by visual inspection against and black and white background for 5 seconds under illumination between about 2000 and about 3750 lux in accordance with USP <790> after reconstitution and dilution of the therapeutic or diagnostic agent to a standard concentration, e.g., about 100 mg/mL or about 1 mg/mL. In some embodiments, fewer than 65 samples in 10,000 (0.65%) are rejected on the basis of USP <790>. Alternate inspection strategies are light-obscuration, automated optical imaging systems, or X-ray imaging in accordance with USP <1790>.

In other embodiments, particulate contaminants with characteristic sizes from about 1 μm to about 100 μm persist upon dissolution in an aqueous liquid. In some embodiments, particulate contaminants are present in quantities from about 0 to 100,000,000 per about 1 mL, e.g., from about 0 to about 10,000,000 per about 1 mL, from about 0 to about 1,000,000 per about 1 mL, from about 0 to about 500,000 per about 1 mL, from about 0 to about 100,000 per about 1 mL, from about 0 to about 50,000 per about 1 mL, from about 0 to about 10,000 per about 1 mL, from about 0 to about 6,000 per about 1 mL, from about 0 to about 1,000 per about 1 mL, from about 0 to about 600 per about 1 mL, from about 0 to about 250 per about 1 mL, from about 0 to about 100 per about 1 mL, from about 0 to about 60 per about 1 mL, or from about 0 to about 10 per about 1 mL. In other embodiments, particulate contaminants with characteristic size greater than or equal to 10 μm is from about 0 to about 6,000 per about 1 mL, e.g., from about 0 to about 1,000 per about 1 mL, from about 0 to about 100 per about 1 mL, from about 0 to about 10 per about 1 mL, from about 0 to about 5 per about 1 mL, from about 0 to about 3 per about 1 mL, or from about 0 to about 1 per about 1 mL. In certain embodiments, particulate contaminants with characteristic size greater than or equal to 25 μm is from about 0 to about 600 per about 1 mL, e.g., from about 0 to about 100 per about 1 mL, from about 0 to about 10 per about 1 mL, from about 0 to about 3 per about 1 mL, from about 0 to about 1 per about 1 mL, from about 0 to about 0.5 per about 1 mL, or from about 0 to about 0.1 per about 1 mL. Example methods of measuring particulate contaminants include analysis of the therapeutic biologic with a Coulter Counter, HIAC Royco, or micro-flow imaging system after reconstitution and dilution of the therapeutic biologic to a standard concentration, e.g., about 100 mg/mL or about 1 mg/mL. In still other embodiments, the composition has a concentration of particulate contaminants of about 0 per about 1 mL to about 100,000,000 per about 1 mL of greater than about 10 μm particulate contaminants upon dissolution in an aqueous liquid. In certain embodiments, the composition has a concentration of particulate contaminants of about 0 per about 1 mL to about 6000 per about 1 mL of greater than about 10 μm particulate contaminants upon dissolution in an aqueous liquid. In some embodiments, the composition has a concentration of particulate contaminants of about 0 per about 1 mL to about 600 per about 1 mL of greater than about 25 μm particulate contaminants upon dissolution in an aqueous liquid. In other embodiments, the composition is substantially free of particulate contaminants upon dissolution in an aqueous liquid. In some embodiments, the aqueous liquid is water, aqueous buffer or a physiologically relevant aqueous liquid.

In some embodiments, particulate contaminants with characteristic sizes from about 100 nm to about 1 μm persist upon dissolution in an aqueous liquid. In some embodiments, particulate contaminants are present in quantities from about 0 to 5×10¹² per about 1 mL, e.g., from about 0 to about 0.5×10¹² per about 1 mL, from about 0 to about 50×10⁹ per about 1 mL, from about 0 to about 10×10⁹ per about 1 mL, from about 0 to about 5×10⁹ per about 1 mL, from about 0 to about 0.5×10⁹ per about 1 mL, from about 0 to about 50×10⁶ per about 1 mL, from about 0 to about 1×10⁶ per about 1 mL, from about 0 to about 500,000 per about 1 mL, from about 0 to about 200,000 per about 1 mL, from about 0 to about 100,000 per about 1 mL, from about 0 to about 10,000 per about 1 mL, from about 0 to about 5000 per about 1 mL, or from about 0 to about 1000 per about 1 mL. Example methods of measuring particulate contaminants quantitatively include analysis of the agent with a NanoSight, micro-flow imaging system, asymmetric field flow fractionation coupled to a multi-angle laser light scattering (AF4 MALS), or Dynamic Light Scattering (DLS) after reconstitution and dilution of the agent to a standard concentration, e.g., about 100 mg/mL, about 1 mg/mL, or about 1 μg/mL. In some embodiments, particulate contaminants are within a range comparable to the starting monomeric therapeutic biologic solution. In other embodiments, the composition is substantially free of particulate contaminants upon dissolution in an aqueous liquid. In some embodiments, the aqueous liquid is water, aqueous buffer or a physiologically relevant aqueous liquid.

In certain embodiments, particles or a composition comprising particles includes particulate contaminants smaller than or equal to 1 μm. In some embodiments, compositions have a concentration of particulate contaminants with a characteristic size greater than or equal to about 100 nm is about 1 to 5×10¹² per about 1 mL in the composition, or have a concentration of particulate contaminants with a characteristic size less than or equal to about 1 μm is about 1 to 5×10¹² per about 1 mL in the composition. In still other embodiments, the particles or composition of particles may include particulate contaminants larger than or equal to about 1 μm in size. In certain other embodiments, the number of particulate contaminants is from about 0 to about 100,000,000 per about 1 mL, e.g., less than about 10,000,000, 1,000,000, 100,000, 10,000, 1000, 100, 10, or about 1 per about 1 mL. For example, the number of particulate contaminants greater than about 10 μm is from about 0 to about 6,000 per about 1 mL, e.g., less than about 5,000, about 4,000, about 3,000, about 2,000, about 1,000, about 500, about 100, about 10, or about 1 per about 1 mL, and/or the number of particulate contaminants greater than about 25 μm is from about 0 to about 600 per about 1 mL, e.g., less than about 500, about 400, about 300, about 200, about 100, about 50, about 10, or about 1 about 1 per about 1 mL.

In some embodiments, the disclosure provides a composition, e.g., a suspension or dried form, containing a plurality of particles that include an agent, e.g., a therapeutic or diagnostic agent. The composition preferably has a concentration of particulate contaminants of between about 0 and about 100,000,000 per about 1 mL in the composition or upon reconstitution. In other embodiments, the concentration of particulate contaminants is between about 0 and about 1,000,000 per about 1 mL in the composition or upon reconstitution. In still other embodiments, the concentration of particulate contaminants is between about 0 and about 10,000 per about 1 mL in the composition or upon reconstitution. In certain other embodiments, the concentration of particulate contaminants with a characteristic size greater than or equal to about 10 μm is between about 0 to about 6,000 per about 1 mL in the composition or upon reconstitution. In certain embodiments, the concentration of particulate contaminants with a characteristic size greater than or equal to about 25 μm is between about 0 to about 600 per about 1 mL in the composition or upon reconstitution.

In other embodiments, after dissolution or reconstitution of particles following storage, particulate contaminants are present in quantities from about 0 to about 100,000,000 per about 1 mL, e.g., from about 0 to about 10,000,000 per about 1 mL, from about 0 to about 1,000,000 per about 1 mL, from about 0 to about 500,000 per about 1 mL, from about 0 to about 100,000 per about 1 mL, from about 0 to about 50,000 per about 1 mL, from about 0 to about 10,000 per about 1 mL, from about 0 to about 6,000 per about 1 mL, from about 0 to about 1,000 per about 1 mL, from about 0 to about 600 per about 1 mL, from about 0 to about 250 per about 1 mL, from about 0 to about 100 per about 1 mL, from about 0 to about 60 per about 1 mL, or from about 0 to about 10 per about 1 mL. In some embodiments, particulate contaminants with characteristic size greater than or equal to about 10 μm is from about 0 to about 6,000 per about 1 mL, e.g., from about 0 to about 1,000 per about 1 mL, from about 0 to about 100 per about 1 mL, from about 0 to about 10 per about 1 mL, from about 0 to about 5 per about 1 mL, from about 0 to about 3 per about 1 mL, or from about 0 to about 1 per about 1 mL. In certain embodiments, particulate contaminants with characteristic size greater than or equal to about 25 μm is from about 0 to about 600 per about 1 mL, e.g., from about 0 to about 100 per about 1 mL, from about 0 to about 10 per about 1 mL, from about 0 to about 3 per about 1 mL, from about 0 to about 1 per about 1 mL, from about 0 to about 0.5 per about 1 mL, or from about 0 to about 0.1 per about 1 mL. In still other embodiments, after dissolution or reconstitution of the particles following storage, the therapeutic or diagnostic agent retains from about 0.5 to about 1.0 activity, e.g., from about 0.75 to about 1.0 activity, from about 0.9 to about 1.0 activity, from about 0.95 to about 1.0 activity, from about 0.99 to about 1.0 activity, or from about 0.999 to about 1.0 activity. In certain other embodiments, dissolution or reconstitution of the particles following storage provides less than about a 10% increase in aggregates of the agent, e.g., a protein, (e.g., less than about 8%, less than about 5%, less than about 4%, less than about 3%, less than about 1%, less than about 0.5%, or less than about 0.1%) as compared to the agent in the first liquid prior to processing. In certain embodiments, the dissolution or reconstitution of the particles after storage provides less than about a 10% increase in fragments of the agent, e.g., a protein, (e.g., less than about 8%, less than about 5%, less than about 4%, less than about 3%, less than about 1%, less than about 0.5%, or less than about 0.1%) as compared to the therapeutic or diagnostic agent in the first liquid prior to processing. In some embodiments, the dissolution or reconstitution of the particles following storage provides less than about a 50% change in charge variants in the population of the agent, e.g., an antibody or an antibody fragment, (e.g., less than about 40, 30, 20, 10, 8, 5, 4, 3, or about 1%) as compared to the therapeutic or diagnostic agent prior to particle formation.

In still other embodiments, after dissolution or reconstitution of the particles following storage, particulate contaminants are present in quantities from about 0 to about 100,000,000 per about 1 mL, e.g., from about 0 to about 10,000,000 per about 1 mL, from about 0 to about 1,000,000 per about 1 mL, from about 0 to about 500,000 per about 1 mL, from about 0 to about 100,000 per about 1 mL, from about 0 to about 50,000 per about 1 mL, from about 0 to about 10,000 per about 1 mL, from about 0 to about 6,000 per about 1 mL, from about 0 to about 1,000 per about 1 mL, from about 0 to about 600 per about 1 mL, from about 0 to about 250 per about 1 mL, from about 0 to about 100 per about 1 mL, from about 0 to about 60 per about 1 mL, or from about 0 to about 10 per about 1 mL. In certain embodiments, particulate contaminants with characteristic size greater than or equal to about 10 μm is from about 0 to about 6,000 per about 1 mL, e.g., from about 0 to about 1,000 per about 1 mL, from about 0 to about 100 per about 1 mL, from about 0 to about 10 per about 1 mL, from about 0 to about 5 per 1 mL, from about 0 to about 3 per about 1 mL, or from about 0 to about 1 per about 1 mL. In certain other embodiments, particulate contaminants with characteristic size greater than or equal to about 25 μm is from about 0 to about 600 per about 1 mL, e.g., from about 0 to about 100 per about 1 mL, from about 0 to about 10 per about 1 mL, from about 0 to about 3 per about 1 mL, from about 0 to about 1 per about 1 mL, from about 0 to about 0.5 per about 1 mL, or from about 0 to about 0.1 per about 1 mL. In some embodiments, dissolution or reconstitution of the particles following storage provides less than about a 10% increase in aggregates of the diagnostic or therapeutic agent, e.g., a protein, (e.g., less than about 8%, less than about 5%, less than about 4%, less than about 3%, less than about 1%, less than about 0.5%, or less than about 0.1%) as compared to the therapeutic or diagnostic agent in the first liquid prior to processing. In other embodiments, the dissolution or reconstitution of the particles after storage provides less than about a 10% increase in fragments of the diagnostic or therapeutic agent, e.g., a protein, (e.g., less than about 8%, less than about 5%, less than about 4%, less than about 3%, less than about 1%, less than about 0.5%, or less than about 0.1%) as compared to the therapeutic or diagnostic agent in the first liquid prior to processing. In certain other embodiments, the dissolution or reconstitution of the particles following storage provides less than about 50% change in charge variants in the population of a diagnostic or therapeutic agent, e.g., an antibody or an antibody fragment, (e.g., less than about 40, about 30, about 20, about 10, about 8, about 5, about 4, about 3, or about 1%) as compared to the therapeutic or diagnostic agent prior to particle formation.

In certain embodiments, the particles of the disclosure can be suspended in an aqueous liquid, an organic liquid, an ionic liquid, a gel, or a combination thereof to form a composition (e.g., suspension formulation). The medium for the composition (e.g., suspension) may further include, e.g., a carbohydrate, a pH adjusting agent, a salt, a chelator, a mineral, a polymer, a surfactant, an amino acid, an oligopeptide, a biologic excipient, a chemical excipient, an antiseptic, an antioxidant, a paraben, a bactericide, a fungicide, a vitamin, a preservative, an analgesic, and/or nutrient media. In some embodiments, each of the other components is, independently, at about 0.0001 to about 99% (w/v) of the medium, e.g., at about 0.0001 to about 90% (w/v), at about 0.0001 to about 50% (w/v), at about 0.0001 to about 10% (w/v), at about 0.0001 to about 1% (w/v), or at about 0.0001 to about 0.1% (w/v).

For compositions such as aqueous suspension formulations, high concentration trehalose solutions can stabilize the particles in the composition (e.g., suspension) and prevent premature dissolution. The sugar acts as a steric stabilizer if adsorbed onto the particle surface but if non-absorbing can also act as a “crowder” molecule. A crowder molecule may function by enhancing depletion repulsions. This stabilizing effect has also been described for other crowding agents in water such as (i) polymers, e.g., PEG 200, PEG 300, PEG 3350, PEG 8000, PEG 10000, PEG 20000, polyoxamers, polyvinylpyrrolidone, polyacrylic acids, poly(vinyl) polymers, polyesters, polyaldehydes, tert-polymers, polyamino acids, and hydroxyethylstarch, etc. (the foregoing may be used alone or in combination); (ii) organic molecules, e.g., N-methyl-2-pyrrolidone (Miller et al. J. Pharm. Sci., 2012, 101, 3763-3778), and (iii) sugars and sugar alcohols such as sorbitol, sucrose, and mannitol, among others. Other “crowding agents” include salts such as ammonium sulfate which can compete for water of hydration, and water soluble organic liquids such as N-methyl pyrrolidone (NMP) which can lower the solvent dielectric constant and produce excluded volume effects. In preferred embodiments, the crowding agent is PEG 3350, Dextran 40k, or Dextran 6k.

In some embodiments, the surfactant in a composition comprising a suspension liquid (either aqueous and non-aqueous) acts as a charge stabilizer. The surfactant adsorbs onto the surface of the particles to control electrostatic interactions between them. The repulsive electrostatic force generated upon the addition of surfactant to the composition (e.g., suspension) is sufficient in some embodiments to prevent significant aggregation of the particles. The surfactant can also prevent attachment to the container. In other embodiments, a polymer can be added to the composition to act as a steric stabilizer.

In certain embodiments, the therapeutic or diagnostic agent has about 0.5 to about 1.0 activity per unit, e.g., about 0.75 to about 1.0 activity per unit, or about 0.9 to about 1.0 activity per unit (e.g., about 0.99 activity per unit).

In certain preferred embodiments, the present disclosure as described herein, concerns a highly concentrated composition comprising a plurality of particles comprising at least one therapeutic biologic suspended in a low viscosity pharmaceutically acceptable liquid carrier, wherein the composition upon dissolution in water, buffers or other physiologically relevant aqueous liquids, e.g., biological fluids in the patients' body, have a substantially similar turbidity compared to a similar aqueous composition comprising monomeric therapeutic biologics. The term “turbidity” means the cloudiness or haziness of a fluid caused by individual particles that remain insoluble after dissolution at the desired concentration in water, buffer or other physiologically relevant aqueous liquids, e.g., biological fluids in the patients' body. As used herein, “physiologically relevant” conditions as may be encountered inside a mammal or human, can apply. The skilled person will be able to determine the set of conditions most appropriate for testing in accordance with the ultimate application of the compositions described herein. In some embodiments, the composition upon dissolution in an aqueous liquid has a substantially similar turbidity compared to an aqueous composition comprising monomeric therapeutic biologics. In preferred embodiments, the composition upon dissolution in an aqueous liquid is substantially free of turbidity. In certain preferred embodiments, the aqueous liquid is water, aqueous buffer or a physiologically relevant aqueous liquid.

In some embodiments, the particles of the disclosure can be reconstituted into a liquid pharmaceutical composition to assess the turbidity or turbidance (USP <855>). Turbidity may be measured in units of FTU (Formazin Turbidity Units). This is achieved by comparing the turbidity of a sample with that of a formazine suspension. Turbidity may also be measured as Nephelometric Turbidity Units (NTU) where 1 NTU=1 FTU. In other embodiments, when 10 mg of particles are dissolved in 1 mL of liquid, turbidity can be between about 0 to about 4000 FTU, about 0 to about 1000 FTU, about 0 to about 500 FTU, about 0 to about 50 FTU, about 0 to about 20 FTU, about 0 to about 10 FTU, about 0 to about 5 FTU, about 0 to about 1 FTU, about 0 to about 0.1 FTU, or about 0 to about 0.01 FTU. In certain embodiments, the composition has a turbidity of between about 0 to about 4000 Formazin Turbidity Units (FTU). In certain other embodiments, the composition upon dissolution in an aqueous liquid has a substantially similar turbidity compared to an aqueous composition comprising the therapeutic biologic in monomeric form. In preferred embodiments, the composition upon dissolution in an aqueous liquid is substantially free of turbidity. In certain preferred embodiments, the aqueous liquid is water, aqueous buffer or a physiologically relevant aqueous liquid.

In other embodiments, the disclosure concerns highly concentrated compositions of low turbidity comprising a carbohydrate, a pH adjusting agent, a salt, a surfactant, a protein stabilizer, an emulsifier, an amino acid, and a plurality of particles comprising a therapeutic biologic, in a non-aqueous liquid carrier. In preferred embodiments, the disclosure concerns highly concentrated compositions of low turbidity comprising trehalose, arginine hydrochloride, sodium succinate, succinic acid, citric acid, sodium citrate, histidine, histidine hydrochloride, sodium chloride, hydroxypropyl beta-cyclodextrin, polysorbate, polysorbate 80 or sorbitan monooleate, and a plurality of particles comprising an antibody, in ethyl oleate. In certain preferred embodiments, the composition upon dissolution in water, aqueous buffer or any physiologically relevant aqueous liquid is substantially free of turbidity.

The composition comprising a plurality of particles comprising at least one therapeutic biologic described herein, can be prepared in a number of ways, as well as any methods of forming the particles disclosed in, for example, PCT/US2021/027755, PCT/US2020/015957, PCT/US2017/063150, PCT/US2018/043774, PCT/US2019/033875, and U.S. 62/799,696, each of which is hereby incorporated by reference in its entirety.

### Droplets

Droplets as described herein, can be formed through any of several techniques that are known in the art. These include rotary atomization, pneumatic atomization, ultrasonic atomization, sonic atomization, vibrating mesh nebulization, jet atomization, microfluidic droplet generation, flow focusing, membrane emulsification, electrospray, or homogenization. The term “droplet” or “droplets” or “drops” refer to a material that has a liquid outer surface. In certain embodiments, the droplets of step a) are formed by electrospray, an ultrasonic atomizer, or a microfluidic device. In preferred embodiments, the droplets of step a) are formed in a microfluidic device. In certain preferred embodiments, the droplets formed in the microfluidic device are regularly spaced in the microfluidic device.

The term “feed solution” refers to a preparation of the therapeutic or diagnostic agents in the first liquid, either as a solution, a slurry, or some other liquid form. In some embodiments, the preparation contains excipients. In other embodiments, the preparation further contains a buffer.

In some embodiments, the first liquid is aqueous, an organic solvent, an ionic liquid, a hydrogel, an ionogel, or a combination thereof. In other embodiments, the first liquid is aqueous. In certain embodiments, the first liquid is water, 0.9% saline, lactated Ringer's solution, buffers, dextrose 5%, or a combination thereof. In certain other embodiments, the buffer is acetate buffer, histidine buffer, succinate buffer, HEPES buffer, tris buffer, carbonate buffer, citrate buffer, phosphate buffer, phosphate-buffered saline, glycine buffer, barbital buffer, cacodylate buffer, ammonium formate buffer, urea solution, or a combination thereof. In preferred embodiments, the first liquid is water.

In other embodiments, the organic liquid is acetone, acetonitrile, acyclic alkanes (e.g., hexanes, heptane, pentane), amyl acetate, butanol, butyl acetate, chlorobenzene, chloroform, cumene, cyclohexane, 1,2-dichloroethene, dichloromethane, diethyl ether, dimethoxyethane, dimethylacetamide, dimethylformamide, dimethyl sulfoxide, 1,4-dioxane, ethanol, 2-ethoxyethanol, ethyl acetate, ethyl nitrate, ethyleneglycol, hydrazine, isopropanol, methanol, methyl acetate, 2-methyl-1-butanol, 2-methyl-1-propanol, methylbutyl ketone, methylcyclohexane, methylethyl ketone, methylpyrrolidone, methyl tert-butyl ether, nitromethane, propanol, propyl acetate, sulfolane, propyleneglycol, tetrahydrofuran, tetralin, toluene, 1,1,2-tricholoroethane, triethylamine, xylene, benzyl benzoate, ethyl lactate, dimethyl isosorbide, dimethyl sulfoxide, glycofurol, diglyme, methyl tert-butyl ether, polyethylene glycol, 2-pyrrolidone, tetrahydrofurfuryl alcohol, trigylcerides, octyl acetate, ethanol, butanol, octanol, decanol, diglyme, tocopherol, octa-fluoropropane, (perfluorohexyl) octane, n-acetyltryptophan, trigylcerides, triglycerides of the fractionated plant fatty acids C8 and C10, propylene glycol diesters (e.g., propylene glycol diesters of saturated plant fatty acids C8 and C10), ethyl laurate, methyl caprylate, methyl caprate, methyl myristate, methyl oleate, methyl linoleate, dimethyl adipate, dibutyl suberate, diethyl sebacate, ethyl macadamiate, trimethylolpropane triisosterate, isopropyl laurate, isopropyl myristate, diethyl succinate, polysorbate esters, ethanol amine, propanoic acid, triacetin, citral, anisole, anethol, benzaldehyde, linalool, caprolactone, phenol, thioglycerol, dimethylacetamide, ethyl formate, ethyl hexyl acetate, eugenol, clove bud oil, diethyl glycol monoether, dimethyl isosorbide, isopropyl acetate, methyl isobutyl ketone, methyl tert-butyl ether, N-methyl pyrrolidone, perfluorodecalin, 2-pyrrolidone, ethyl oleate, ethyl caprate, dibutyl adipate, hexanoic acid, octanoic acid, diethyl glycol monoether, gamma-butyrolactone, polyoxyl 40 hydrogenated castor oil, polyoxyl 35 castor oil, propylene carbonate, octanol, hexanol, sorbitan monooleate, n-acetyltryptophan, solketal, an alkyl acetate, an aryl acetate, an aryl alkyl acetate, tolyl acetate, benzyl acetate, polysorbate 80, phenethyl acetate, phenyl acetate, glycerol, or a combination thereof. In other embodiments, the first liquid is an oil. In certain embodiments, the oil is coconut oil, cottonseed oil, fish oil, grape seed oil, hazelnut oil, hydrogenated vegetable oils, lime oil, olive oil, palm seed oil, peanut oil, peppermint oil, safflower oil, sesame oil, soybean oil, sunflower oil, walnut oil, silicon oil, mineral oil, or a combination thereof. In still other embodiments, the first liquid is an ionic liquid. In certain other embodiments, the ionic liquid contains (i) cations such as pyridinium, pyridazinium, pyrimidinium, pyrazinium, imidazolium, pyrazolium, thiazolium, oxazolium, triazolium, ammonium, sulfonium; and (ii) anions such as halides, sulfates, sulfonates, carbonates, phosphates, bicarbonates, nitrates, acetates, PF₆-, BF₄-, triflate, nonaflate, bis(triflyl)amide, trifluoroacetate, heptafluorobutanoate, haloaluminate, or a combination thereof.

In certain embodiments, the first liquid is a hydrogel, an ionogel, or a combination thereof. Exemplary hydrogels are prepared from polymers such as collagen, chitosan, methylcellulose, dextran, alginate, agarose, poly(methyl methacrylate), poly(amido amine), poly(ethyleneimine), polyethylene oxide, gelatin, hyaluronic acid, or a combination thereof, and may contain water, aqueous solutions, and other polar solvents. Exemplary organogels are prepared form organogelators such as 4-tert-butyl-1-aryl cyclohexanols, L-lysine derivatives, poly(ethylene glycol), polycarbonate, polyesters, polyalkenes, oxalyl amide derivatives containing alkyl ester groups, or low molecular weight compounds such as fatty acids and n-alkanes, and contain a non-polar solvent phase. Ionogels are analogous to organogels with the exception that the solvent phase is an ionic liquid.

In some embodiments, the concentration of the therapeutic agent in the first liquid as described herein, is about 10 mg/mL to about 650 mg/mL, e.g., about 20, 30, 50, 75, 100, 125, 150, 175, 200, 225, 250, 275, 300, 325, 350, 375, 400, 425, 450, 475, 500, 525, 550, 575, 600, 625 mg/mL to about 650 mg/mL; about 20 mg/mL to about 625 mg/mL, e.g., about 20, 30, 50, 75, 100, 125, 150, 175, 200, 225, 250, 275, 300, 325, 350, 375, 400, 425, 450, 475, 500, 525, 550, 575, 600 mg/mL to about 625 mg/mL; about 20 mg/mL to about 600 mg/mL, e.g., about 20, 30, 50, 75, 100, 125, 150, 175, 200, 225, 250, 275, 300, 325, 350, 375, 400, 425, 450, 475, 500, 525, 550, 575 mg/mL to about 600 mg/mL; about 20 mg/mL to about 575 mg/mL, e.g., about 20, 30, 50, 75, 100, 125, 150, 175, 200, 225, 250, 275, 300, 325, 350, 375, 400, 425, 450, 475, 500, 525, 550 mg/mL to about 575 mg/mL; about 20 mg/mL to about 550 mg/mL, e.g., about 20, 30, 50, 75, 100, 125, 150, 175, 200, 225, 250, 275, 300, 325, 350, 375, 400, 425, 450, 475, 500, 525 mg/mL to about 550 mg/mL; about 20 mg/mL to about 525 mg/mL, e.g., about 20, 30, 50, 75, 100, 125, 150, 175, 200, 225, 250, 275, 300, 325, 350, 375, 400, 425, 450, 475, 500 mg/mL to about 525 mg/mL; about 20 mg/mL to about 500 mg/mL, e.g., about 20, 30, 50, 75, 100, 125, 150, 175, 200, 225, 250, 275, 300, 325, 350, 375, 400, 425, 450, 475 mg/mL to about 500 mg/mL; about 20 mg/mL to about 475 mg/mL, e.g., about 20, 30, 50, 75, 100, 125, 150, 175, 200, 225, 250, 275, 300, 325, 350, 375, 400, 425, 450 mg/mL to about 475 mg/mL; about 20 mg/mL to about 450 mg/mL, e.g., about 20, 30, 50, 75, 100, 125, 150, 175, 200, 225, 250, 275, 300, 325, 350, 375, 400, 425 mg/mL to about 450 mg/mL; about 20 mg/mL to about 425 mg/mL, e.g., about 20, 30, 50, 75, 100, 125, 150, 175, 200, 225, 250, 275, 300, 325, 350, 375, 400 mg/mL to about 425 mg/mL; about 20 mg/mL to about 400 mg/mL, e.g., about 20, 30, 50, 75, 100, 125, 150, 175, 200, 225, 250, 275, 300, 325, 350, 375 mg/mL to about 400 mg/mL; about 20 mg/mL to about 375 mg/mL, e.g., about 20, 30, 50, 75, 100, 125, 150, 175, 200, 225, 250, 275, 300, 325, 350 mg/mL to about 375 mg/mL; about 20 mg/mL to about 350 mg/mL, e.g., about 20, 30, 50, 75, 100, 125, 150, 175, 200, 225, 250, 275, 300, 325 mg/mL to about 350 mg/mL; about 20 mg/mL to about 325 mg/mL, e.g., about 20, 30, 50, 75, 100, 125, 150, 175, 200, 225, 250, 275, 300 mg/mL to about 325 mg/mL; or about 20 mg/mL to about 300 mg/mL, e.g., about 20, 30, 50, 75, 100, 125, 150, 175, 200, 225, 250, 275 mg/mL to about 300 mg/mL. In other embodiments, the concentration of the therapeutic agent in the first liquid is about 10 mg/mL to about 500 mg/mL. In certain embodiments, the concentration of the therapeutic agent in the first liquid is about 10 mg/mL to about 100 mg/mL. In preferred embodiments, the concentration of the therapeutic agent in the first liquid is about 20 mg/mL to about 100 mg/mL. In other embodiments of the disclosure, the concentration of the therapeutic or diagnostic agent in the first liquid is from about 0.0001 to about 1000 mg/mL, e.g., about 100 to about 800, about 200 to about 700, about 200 to about 600, or about 300 to about 700 mg/mL. In still other embodiments, the particles have a mass loading of the therapeutic or diagnostic agent from about 1% to about 100%.

In other embodiments, the first liquid has a viscosity of less than about 200 mPa·s, less than about 150 mPa·s, less than about 125 mPa·s, less than about 100 mPa·s, less than about 75 mPa·s, less than about 75 mPa·s, less than about 70 mPa·s, less than about 65 mPa·s, less than about 60 mPa·s, less than about 55 mPa·s, less than about 50 mPa·s, less than about 45 mPa·s, less than about 40 mPa·s, less than about 35 mPa·s, less than about 30 mPa·s, less than about 25 mPa·s, less than about 20 mPa·s, less than about 19 mPa·s, less than about 18 mPa·s, less than about 17 mPa·s, less than about 16 mPa·s, less than about 15 mPa·s, less than about 14 mPa·s, less than about 13 mPa·s, less than about 12 mPa·s, less than about 11 mPa·s, less than about 10 mPa·s, less than about 9.5 mPa·s, less than about 9 mPa·s, less than about 8.5 mPa·s, less than about 8 mPa·s, less than about 7.5 mPa·s, less than about 7 mPa·s, less than about 6.5 mPa·s, less than about 6 mPa·s, less than about 5.5 mPa·s, less than about 5 mPa·s, less than about 4.5 mPa·s, less than about 4 mPa·s, less than about 3.5 mPa·s, less than about 3 mPa·s, less than about 2.5 mPa·s, less than about 2 mPa·s, less than about 1.5 mPa·s, less than about 1 mPa·s, less than about 0.5 mPa·s, less than about 0.1 mPa·s, less than about 0.05 mPa·s, or less than about 0.01 mPa·s (one millipascal-second). In other embodiments, the first liquid has a viscosity of about 0.01 mPa·s to about 10,000 mPa·s, e.g., from about 0.01 mPa·s to about 1,000 mPa·s, from about 0.01 mPa·s to about 100 mPa·s, from about 0.01 mPa·s to about 50 mPa·s, from about 0.01 mPa·s to about 25 mPa·s, from about 0.01 mPa·s to about 10 mPa·s, from about 0.01 mPa·s to about 5 mPa·s, or from about 0.01 mPa·s to about 1 mPa·s. In certain embodiments, the first liquid has a viscosity that can range from about 0.27 mPa·s to about 200 mPa·s, e.g., about 0.27 mPa·s to about 50 mPa·s, about 1 mPa·s to about 30 mPa·s, or about 20 mPa·s to about 50 mPa·s. In still other embodiments, the first liquid has a viscosity that ranges from about 0.27 mPa·s to about 200 mPa·s, e.g., about 0.27 mPa·s to about 100 mPa·s, about 0.27 mPa·s to about 50 mPa·s, about 0.27 mPa·s to about 30 mPa·s, about 1 mPa·s to about 20 mPa·s, or about 1 mPa·s to about 15 mPa·s. Methods of controlling viscosity include temperature regulation and viscosity modifying additives. Mixtures of liquids may also be used to control viscosity.

In some embodiments, the first liquid has a viscosity from about 0.01 to about 10,000 mPa·s. In other embodiments, the first liquid has a viscosity of less than about 100 mPa·s. In still other embodiments, the first liquid has a viscosity of less than about 10 mPa·s. In certain other embodiments, the first liquid has a viscosity of less than about 3 mPa·s. In certain embodiments, the first liquid has a viscosity of less than about 0.9 mPa·s. In preferred embodiments, the first liquid has a viscosity of less than about 0.5 mPa·s.

In certain embodiments, the first liquid further comprises a surfactant.

In some embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, or a combination thereof. In certain embodiments, the surfactant is polysorbate, docusate or lecithin. In preferred embodiments, the surfactant is polysorbate 20, polysorbate 60, or polysorbate 80. In certain preferred embodiments, the surfactant is polysorbate 20 or polysorbate 80. In certain other embodiments, the fatty acid ester of sorbitol is a sorbitan ester, e.g., span 20, span 40, span 60, or span 80.

In other embodiments, the second liquid is aqueous, an organic solvent, an ionic liquid, a hydrogel, ionogel, protein stabilizer, or a combination thereof. In some embodiments, the second liquid is aqueous. In preferred embodiments, the second liquid is an organic solvent.

In some embodiments, the organic solvent is benzyl alcohol, benzyl benzoate, castor oil, coconut oil, corn oil, cottonseed oil, fish oil, grape seed oil, hazelnut oil, hydrogenated palm seed oil, olive oil, peanut oil, peppermint oil, safflower oil, sesame oil, soybean oil, sunflower oil, vegetable oil, walnut oil, polyethylene glycol, glycofurol, acetone, diglyme, dimethylacetamide, dimethyl isosorbide, dimethyl sulfoxide, ethanol, ethyl acetate, butyl acetate, ethyl ether, ethyl lactate, isopropyl acetate, methyl acetate, methyl isobutyl ketone, methyl tert-butyl ether, N-methyl pyrrolidone, perfluorodecalin, 2-pyrrolidone, trigylcerides, tetrahydrofurfuryl alcohol, triglycerides of the fractionated plant fatty acids C8 and C10 (e.g., MIGLYOL® 810 and MIGLOYL® 812N), propylene glycol diesters (e.g., propylene glycol diesters of of saturated plant fatty acids C8 and C10 (e.g., MIGLYOL® 840)), ethyl oleate, ethyl caprate, dibutyl adipate, fatty acid esters, hexanoic acid, octanoic acid, triacetin, diethyl glycol monoether, gamma-butyrolactone, eugenol, clove bud oil, citral, limonene, or a combination thereof. In certain embodiments, the organic solvent is ethyl acetate or butyl acetate.

In still other embodiments, the organic solvent is acetone, acetonitrile, acyclic alkanes (e.g., hexanes, heptane, pentane), amyl acetate, butanol, butyl acetate, chlorobenzene, chloroform, cumene, cyclohexane, 1,2-dichloroethene, dichloromethane, diethyl ether, dimethoxyethane, dimethylacetamide, dimethylformamide, dimethyl sulfoxide, 1,4-dioxane, ethanol, 2-ethoxyethanol, ethyl acetate, ethyl nitrate, ethyleneglycol, hydrazine, isopropanol, methanol, methyl acetate, 2-methyl-1-butanol, 2-methyl-1-propanol, methylbutyl ketone, methylcyclohexane, methylethyl ketone, methylpyrrolidone, methyl tert-butyl ether, nitromethane, propanol, propyl acetate, sulfolane, propyleneglycol, tetrahydrofuran, tetralin, toluene, 1,1,2-tricholoroethane, triethylamine, xylene, benzyl benzoate, ethyl lactate, dimethyl isosorbide, dimethyl sulfoxide, glycofurol, diglyme, methyl tert-butyl ether, polyethylene glycol, 2-pyrrolidone, tetrahydrofurfuryl alcohol, trigylcerides, octyl acetate, ethanol, butanol, octanol, decanol, diglyme, tocopherol, octa-fluoropropane, (perfluorohexyl) octane, n-acetyltryptophan, trigylcerides, triglycerides of the fractionated plant fatty acids C8 and C10, propylene glycol diesters (e.g., propylene glycol diesters of saturated plant fatty acids C8 and C10), ethyl laurate, methyl caprylate, methyl caprate, methyl myristate, methyl oleate, methyl linoleate, dimethyl adipate, dibutyl suberate, diethyl sebacate, ethyl macadamiate, trimethylolpropane triisosterate, isopropyl laurate, isopropyl myristate, diethyl succinate, polysorbate esters, ethanol amine, propanoic acid, triacetin, citral, anisole, anethol, benzaldehyde, linalool, caprolactone, phenol, thioglycerol, dimethylacetamide, ethyl formate, ethyl hexyl acetate, eugenol, clove bud oil, diethyl glycol monoether, dimethyl isosorbide, isopropyl acetate, methyl isobutyl ketone, methyl tert-butyl ether, N-methyl pyrrolidone, perfluorodecalin, 2-pyrrolidone, ethyl oleate, ethyl caprate, dibutyl adipate, hexanoic acid, octanoic acid, diethyl glycol monoether, gamma-butyrolactone, polyoxyl 40 hydrogenated castor oil, polyoxyl 35 castor oil, propylene carbonate, octanol, hexanol, sorbitan monooleate, n-acetyltryptophan, solketal, an alkyl acetate, an aryl acetate, an aryl alkyl acetate, tolyl acetate, benzyl acetate, polysorbate 80, phenethyl acetate, phenyl acetate, glycerol, or a combination thereof.

In certain embodiments, the organic solvent is acetonitrile, chlorobenzene, chloroform, cyclohexane, cumene, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, methylisobutylketone, N-methylpyrrolidone, nitromethane, pyridine, sulfolane, tetrahydrofuran, tetralin, toluene, 1,1,2-trichloroethene, xylene, acetic acid, acetone, anisole, 1-butanol, 2-butanol, butylacetate, tert-butylmethyl ether, dimethyl sulfoxide, ethanol, ethylacetate, ethyl ether, ethyl formate, formic acid, heptane, isobutylacetate, isopropylacetate, methylacetate, 3-methyl-1-butanol, methylethyl ketone, 2-methyl-1-propanol, pentane, 1-pentanol, 1-propanol, 2-propanol, propylacetate, triethylamine, 1,1-diethoxypropane, 1,1-dimethoxymethane, 2,2-dimethoxypropane, isooctane, isopropyl ether, methylisopropyl ketone, methyltetrahydrofuran, petroleum ether, trichloroacetic acid, trifluoroacetic acid, decanol, 2-ethylhexylacetate, amylacetate, or a combination thereof.

In some embodiments, the second liquid is an ionic liquid. In certain embodiments, the second liquid is a protein stabilizer.

In other embodiments, the second liquid has a viscosity of less than about 200 mPa·s, less than about 150 mPa·s, less than about 125 mPa·s, less than about 100 mPa·s, less than about 75 mPa·s, less than about 75 mPa·s, less than about 70 mPa·s, less than about 65 mPa·s, less than about 60 mPa·s, less than about 55 mPa·s, less than about 50 mPa·s, less than about 45 mPa·s, less than about 40 mPa·s, less than about 35 mPa·s, less than about 30 mPa·s, less than about 25 mPa·s, less than about 20 mPa·s, less than about 19 mPa·s, less than about 18 mPa·s, less than about 17 mPa·s, less than about 16 mPa·s, less than about 15 mPa·s, less than about 14 mPa·s, less than about 13 mPa·s, less than about 12 mPa·s, less than about 11 mPa·s, less than about 10 mPa·s, less than about 9.5 mPa·s, less than about 9 mPa·s, less than about 8.5 mPa·s, less than about 8 mPa·s, less than about 7.5 mPa·s, less than about 7 mPa·s, less than about 6.5 mPa·s, less than about 6 mPa·s, less than about 5.5 mPa·s, less than about 5 mPa·s, less than about 4.5 mPa·s, less than about 4 mPa·s, less than about 3.5 mPa·s, less than about 3 mPa·s, less than about 2.5 mPa·s, less than about 2 mPa·s, less than about 1.5 mPa·s, less than about 1 mPa·s, less than about 0.5 mPa·s, less than about 0.1 mPa·s, less than about 0.05 mPa·s, or less than about 0.01 mPa·s (one millipascal-second). In other embodiments, the second liquid has a viscosity of about 0.01 mPa·s to about 10,000 mPa·s, e.g., from about 0.01 mPa·s to about 1,000 mPa·s, from about 0.01 mPa·s to about 100 mPa·s, from about 0.01 mPa·s to about 50 mPa·s, from about 0.01 mPa·s to about 25 mPa·s, from about 0.01 mPa·s to about 10 mPa·s, from about 0.01 mPa·s to about 5 mPa·s, or from about 0.01 mPa·s to about 1 mPa·s. In certain embodiments, the second liquid has a viscosity that can range from about 0.27 mPa·s to about 200 mPa·s, e.g., about 0.27 mPa·s to about 50 mPa·s, about 1 mPa·s to about 30 mPa·s, or about 20 mPa·s to about 50 mPa·s. In still other embodiments, the second liquid has a viscosity that ranges from about 0.27 mPa·s to about 200 mPa·s, e.g., about 0.27 mPa·s to about 100 mPa·s, about 0.27 mPa·s to about 50 mPa·s, about 0.27 mPa·s to about 30 mPa·s, about 1 mPa·s to about 20 mPa·s, or about 1 mPa·s to about 15 mPa·s. Methods of controlling viscosity include temperature regulation and viscosity modifying additives. Mixtures of liquids may also be used to control viscosity.

In certain embodiments, the second liquid further comprises a surfactant. In still other embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, or a combination thereof.

In some embodiments, the second liquid has a viscosity from about 0.01 to about 10,000 mPa·s. In other embodiments, the second liquid has a viscosity of less than about 10 mPa·s. In still other embodiments, the second liquid has a viscosity of less than about 5 mPa·s. In certain other embodiments, the second liquid has a viscosity of less than about 2 mPa·s. In certain embodiments, the second liquid has a viscosity of less than about 0.70 mPa·s. In preferred embodiments, the second liquid has a viscosity of less than about 0.40 mPa·s.

The droplets as described herein, may include a first liquid and one or more agents, e.g., a therapeutic and/or diagnostic agent. In certain embodiments, the therapeutic agent is a therapeutic biologic. In still other embodiments, the therapeutic biologic has an activity per unit of about 0.5 to about 1.0. In certain other embodiments, the concentration of the agent, e.g., a therapeutic or diagnostic agent, in the first liquid can be in the range of about 0.0001 to about 1000 mg/mL, e.g., about 100 to about 900 mg/mL, about 200 to about 800 mg/mL, about 200 to about 700 mg/mL, about 200 to about 600 mg/mL, or about 300 to about 500 mg/mL.

In some embodiments, the first liquid is aqueous or an organic solvent, and the second liquid is an oil, aqueous, or an ionic liquid. In other embodiments, the first liquid and/or the second liquid has a viscosity from about 0.01 mPa·s to about 10,000 mPa·s. In certain embodiments, the second liquid is a mixture of two or more liquids of different polarities, where the mixture includes liquids that have differing solubility with the first liquid. In still other embodiments, the first liquid or second liquid further includes a carbohydrate, a pH adjusting agent, a salt, a chelator, a mineral, a polymer, a surfactant, a protein stabilizer, an emulsifier, an antiseptic, an amino acid, an antioxidant, a protein, an organic solvent, a paraben, a bactericide, a fungicide, a vitamin, a preservative, a nutrient media, or a combination thereof. The term “polarity” or “polarities” refer to the overall solvation capability (solvation power) of the solvent, which in turn depends on the action of all possible, nonspecific and specific, intermolecular interactions between solute ions or molecules and solvent molecules, excluding, however, those interactions leading to definite chemical alterations of the ions of molecules of the solute (Chem. Rev., 1994, 94, 2319-2358). A prediction of solvent polarity may be made from their dielectric constant. Solvents with high dielectric constants are considered more polar and those with low dielectric constants are considered less polar or nonpolar (<˜15).

In other embodiments, each of the other components is, independently, about 0.0001 to about 99% (w/v) of the first liquid, e.g., about 0.0001 to about 90% (w/v), about 0.0001 to about 50% (w/v), about 0.0001 to about 10% (w/v), about 0.0001 to about 1% (w/v), or about 0.0001 to about 0.1% (w/v). In certain embodiments, the amount of additional compound, i.e., excipient, present in the first liquid, second, liquid, or medium, is as shown Table 2.

TABLE 2

| Excipient | Range 1 | Range 2 | Range 3 | Range 4 |
|---|---|---|---|---|
| Carbohydrate | 10-30% | 3-50% | 1-80% | 0.3-99% |
| pH adjusting agent | 0.5-5% | 0.2-40% | 0.05-70% | 0.01-99% |
| Salt | 10-50% | 3-70% | 1-85% | 0.3-99% |
| Chelator | 0.01-1% | 0.003-40% | 0.001-80% | 0.0003-99% |
| Mineral | 10-50% | 3-70% | 1-80% | 0.3-99% |
| Polymer | 10-60% | 3-75% | 1-85% | 0.3-99% |
| Surfactant | .01-1% | 0.003-40% | 0.001-80% | 0.0003-99% |
| Amino acids | 10-25% | 3-50% | 1-85% | 0.3-99% |
| Oligopeptide | 10-25% | 3-50% | 1-85% | 0.3-99% |
| Biologic | 10-70% | 3-70% | 1-85% | 0.3-99% |
| Chemical | 10-50% | 3-70% | 1-85% | 0.3-99% |
| Antiseptic | .5-10% | 0.2-50% | 0.05-70% | 0.02-99% |
| Antioxidant | 0.01-1% | 0.003-40% | 0.001-80% | 0.0003-99% |
| Paraben | 0.01-5% | 0.005-10% | 0.001-50% | 0.001-99% |
| Bactericide | 0.01-5% | 0.005-10% | 0.001-50% | 0.001-99% |
| Fungicide | 0.01-5% | 0.005-10% | 0.001-50% | 0.001-99% |
| Vitamin | 1-50% | 1-70% | 0.1-85% | 0.01-99% |
| Preservative | 10-50% | 3-70% | 1-85% | 0.3-99% |
| Analgesic | 0.01-5% | 0.005-10% | 0.001-50% | 0.001-99% |
| Nutrient media | 10-50% | 3-70% | 1-85% | 0.3-99% |
| Organic liquid | 0.001-2% | 0.0003-1% | 0.0001-10% | 0.00003-99% |

In some embodiments, the cohesive forces (e.g., interfacial tension) on the droplet surface in the second liquid pulls the droplets into a spherical shape which is maintained during the course of drying. In other embodiments, the sphericity of the particles ranges from about 0.1 to about 1, e.g., at least about 0.2, about 0.4, about 0.6, or about 0.8. In certain embodiments, the process can result in uniform particles with high sphericity (about >0.9) and roundness or circularity. Methods of measuring particle sphericity include image analysis of scanning electron micrographs of the particles in which the average roundness is calculated on the basis of the cross-sectional shapes of the particles projected onto the plane of the image. Such roundness or circularity factors can be extended to identify the corresponding sphericity.

In other embodiments, the droplet has a core-shell morphology in the which the first liquid (the droplet “core”) is surrounded by one or more concentric layers of additional liquid (the droplet “shell(s)”), each of which may or may not be defined by a unique set of components and/or a unique concentration of components. Each shell liquid can be an aqueous liquid, an organic liquid, an oil, an ionic liquid, or a combination thereof and include one or more agents, e.g., therapeutic or diagnostic agents. The concentration of the agent, e.g., a therapeutic or diagnostic agent, in a shell liquid can be in the range of about 0.0001 to about 1000 mg/mL, e.g., about 100 to about 900 mg/mL, about 200 to about 800 mg/mL, about 200 to about 700 mg/mL, about 200 to about 600 mg/mL, or about 300 to about 500 mg/mL. The shell liquid can further include, e.g., a carbohydrate, a pH adjusting agent, a salt, a chelator, a mineral, a polymer, a surfactant, an amino acid, an oligopeptide, a biologic excipient, a chemical excipient, an antiseptic, an antioxidant, a paraben, a bactericide, a fungicide, a vitamin, a preservative, an analgesic, and/or nutrient media.

In some embodiments, a surfactant in the first liquid and/or the shell liquid(s) prevents coalescence of the droplets. In other embodiments, an oligopeptide excipient, a protein excipient, and/or the agent(s) themselves, e.g., therapeutic or diagnostic agents, act as surfactants. In other embodiments, one or more of the shell layers is a hydrogel, ionogel, organogel, or some combination thereof.

In certain embodiments the, the droplets are electrically charged. As a fraction of the Rayleigh limit, the droplets may on average be charged from about 0 to about 1, e.g., from about 0.1 to about 1.0, from about 0.2 to about 1.0, from about 0.3 to about 1.0, from about 0.4 to about 1.0, or from about 0.5 to about 1.0. In some embodiments, charging assists in the mitigation of droplet coalescence and/or in the control of various particle properties of interest. These include but are not limited to the morphology, the surface chemistry, and the crystallinity of select components. The term “Rayleigh limit” refers to the specific charge, e.g., in units of Coulombs per kilogram, corresponding to the point at which Coulombic repulsion overcomes the binding forces of surface tension in a drop, leading to Coulomb fission or shedding of charge from the drop through some other mechanism.

### EXAMPLES

### Example 1

Reagents should be prepared for each sample required in addition to enough reagent to run three method blanks at the same replicate number as for suspension samples. Method blanks require the same volume of destructive reagent buffers applied to a sample of carrier liquid of equivalent volume to the suspension sample.

### A) Preparation of Dissolution Media (e.g., 0.5% v/v Polysorbate 20 in 1× Phosphate Buffered Saline (PBS) pH 7.4)

Dissolution media will be specific to each product and described in the associated dissolution protocol and batch release protocol.

Volumes should be scaled according to the number of samples to be run in a single day; this preparation is appropriate for a single sample. The reagent should be prepared fresh on the day of analysis.

1. Add 5 mL of Polysorbate 20 (PS20) directly into 1000 mL PBS bottle using a positive displacement pipette.

2. Mix the PBS container at 60 RPM on the nutating mixer for 10 minutes.

3. Store mixture at room temperature until required in Section C).

B) Preparation of Protein Destructive Reagent (e.g., 10% SDS, 8 M Urea, 10 mM Tris(2-carboxyethyl) phosphine hydrochloride (TCEP))

The reagent should be prepared fresh on the day of analysis to avoid the generation of particles from containers, as well as the degradation of urea which will oxidize.

Volumes should be scaled according to the number of samples to be run in a single day; this preparation is appropriate for nine (9) samples.

1. Add 480 g urea to a 1 L disposable bottle using a funnel and a balance.

2. Add 500 mL of 20% SDS solution to the bottle.

3. Fill bottle to ˜900 mL mark with Water For Injection (WFI) water.

4. Cap bottle and mix for 10 minutes at 60 RPM on a nutating mixer.

5. Place bottle in a hot water bath at 60° C. for 15 minutes or till solution turns clear.

6. If any solid residue is observed at the bottom of bottle, mix at 60 rpm on a nutating mixer until all solids have dissolved.

Freshly prepared urea solutions undergo endothermic dissolution; allow solution to heat longer if urea is not dissolved or if bottle feels cold to the touch.

7. Weigh 2.5 g TCEP using an analytical balance and add it to the bottle.

8. Cap the bottle and mix using a nutating mixer at 60 rpm for 5 minutes.

9. Transfer buffer to a clean 1 L graduated cylinder and add remaining volume to fill to the 1 L mark.

10. Filter with 1 L vacuum disposable filter flask and store at room temperature until use. Do not store at 4° C.

### C) Suspension Sample Dissolution and Membrane Filtration

Sections C) and D) should be performed for method blanks, using the same total volume of reagents for both samples and blanks.

All steps in this section should be performed in a laminar flow hood. While handling samples and containers in the flow hood, ensure good practices by avoiding any handling directly behind open samples to avoid extrinsic contamination.

For suspension samples:

1. Vortex suspension for at least 60 seconds to homogenize sample within vial.

2. De-crimp vial and using a 250 μL positive displacement pipette, transfer 250 μL four times to 125 mL particle free bottle.

3. Leave bottle uncapped.

4. Add 3 mL of dissolution media into vial and close vial with the rubber stopper.

5. Vortex vial for 15 seconds at high speed. Transfer contents of vial to the same particle free bottle from Step 2 using the 250 μL positive displacement pipette.

6. Repeat Steps 1-5 for all suspension samples.

For method blanks:

a. Using a 5 mL disposable syringe, draw 5 mL of PGD into syringe.

b. Fit 0.2 μm Millex PTFE filter onto Luer lock of syringe and eject 3 mL into waste.

c. Filter 1 mL of PGD into a labelled particle free bottle.

d. Repeat previous step 2 more times for replicate analysis.

1. Set up LS17 tubing with the peristaltic pump, ensuring the inlet side of the tubing is submerged in the dissolution buffer bottle.

2. Set up Millipak gold endpoint filter at the end of the tubing, with the tubing running inside the BSC.

3. Set flow rate on pump to 100 mL/min and open vent cap of the filter to prime filter with buffer.

4. Start pump and run the pump till the top of the filter has been filled and fully wetted with buffer. Stop priming when the top of the filter has been filled with buffer with minimal air. Close vent cap of the filter.

5. Setup analytical balance in a BSC and place particle free bottle on balance and tare balance.

6. Set peristaltic pump at 300 mL/min and weigh out 25 g±1 g of dissolution buffer directly into particle free bottle with sample.

7. Repeat the previous step for all samples and blanks.

8. Carefully place all samples without caps on an orbital mixer at 120 rpm for 60 minutes.

9. Repeat Steps 3-5 to prime filter with destructive buffer.

10. Dispense 25 g±1 g of destructive buffer into each bottle using the Millipak gold endpoint filter.

11. Place samples on an orbital mixer at 120 rpm for 90 minutes.

12. Transfer membrane filtration apparatus components to a BSC.

13. Filtration assembly components should be washed again within the BSC using the filterjet with water being pressure pumped through the filter. Ensure to rinse each contact surface making sure water stream is running downwards into a waste container.

14. Wash both sides of the membrane with pressure pumping setup and assemble the filtration apparatus as per FIG. 1 and add a single 0.65 μm PVDF filter membrane to the filtration apparatus (#3 in FIG. 1).

15. Turn on the vacuum and connect the tubing to the filtering cup connector.

16. Pour the entire contents of the dissolved suspension in the 125 mL particle free bottle directly into the filtration apparatus.

17. Wash the tube using the filterjet to remove any particles that may be stuck to the inside of the tube.

Pressure pumping setup may be used to wash vials from the bottom of the vial in a side-to-side motion moving toward the rim of the vial.

18. Apply vacuum to pull any residual water and perform 5× ˜50 mL washes using WFI water.

19. After the water has completely passed through the filter, add 20 mL of reagent alcohol using a 25 mL syringe fitted with the 0.2 μm PTFE filter.

20. Apply vacuum for at least 30 seconds.

21. Remove membrane from filtration apparatus using clean tweezers and place into “PetriSlide for contamination analysis” (CAT/PN: PD1504700) with the lid slightly ajar in the BSC and allow to dry for at least 15 minutes or until dry.

22. After drying, the membrane is moved to the microscope stage and the PetriSlide lid is removed. Collection of micrographs can then proceed as described in the next section. If sample collection cannot occur after membrane drying, the cover of the PetriSlide can be fully closed and stored for future analysis.

### D) Instrument Startup for Zeiss Microscope

Turn on the Zeiss Axio Imager M2 by turning on the two power modules attached to the instrument and pressing the power button on the left side of the instrument.

Select the Zeiss Zen Core imaging software on the desktop to open the main application panel.

If needed go to the maintenance menu to swap the camera to “705 pol”.

### Instrument Workflow Setup for Zeiss Microscope

1. From the main software panel enter job mode and select the job that performs membrane scanning with manual auditing. The current iteration of this method is “Elektrofi Image Capture with Manual Inspection V3”

If prompted to perform an autocalibration in the software, click yes and allow the software to automatically calibrate the stage for xy position and z-focusing.

2. Enter the information in the required fields for populating the final generated report.

3. Click “Exit Loop” when the samples are entered into the form.

Up to 6 samples can be entered into the initial form if samples are to be run using the same measurement frame. Alternatively, the loop can be exited after an individual sample if the frame needs to be reset for each subsequent sample.

4. Ensure the following optical configuration parameters are selected before initiating data collection:
Objective: 10×
Contrast Mode: polarization
Camera: AxioCam 705 Pol

The camera can be changed in the “maintenance” menu that can be found on the home screen of Zen Core.
Exposure Time: 13.000 ms (adjust if needed to achieve adequate image)
Brightness: 55%

The gain may be increased if required for routine analysis; however, increasing the brightness setting above 100% will increase detector noise.

Set tile overlap to 10%, maximal shift to 10%, minimal overlap to 2%, and turn on “Perform Stitching.”

5. Align the scanning field to the edges of the membrane holder. Alignment should be checked in at least 4 positions (i.e., left, right, top, and bottom of the membrane relative to the 2d display direction).

6. Define the shading correction in the post processing section.

7. Define the focal plane field using the Onion Skin Model with 36 points and a margin of 3 frames from the edge.

8. Define the z-distance at each of the support points by using the focusing knob on the microscope after clicking on “verify support points/tile regions”. All 36 points should be assessed for proper focus.

If a region is free of defects, it may be difficult to determine the optimal focus. To achieve a dependable focusing procedure, the F-stop can be closed partially until the aperture edges are visible. Adequate focus is achieved when the aperture edges are also in focus.

9. Apply shading correction under the post processing section. To do this, defocus the image by adjusting the z-position of the stage and click “shading correction.” Ensure that it is set to specific, not global.

If the image appears to have any obvious distortions, repeat Steps 6 through 9.

10. Click run to start image collection.

11. Define the region of interest by aligning the circular region as close to the edge of the membrane holder as possible without touching the edge.

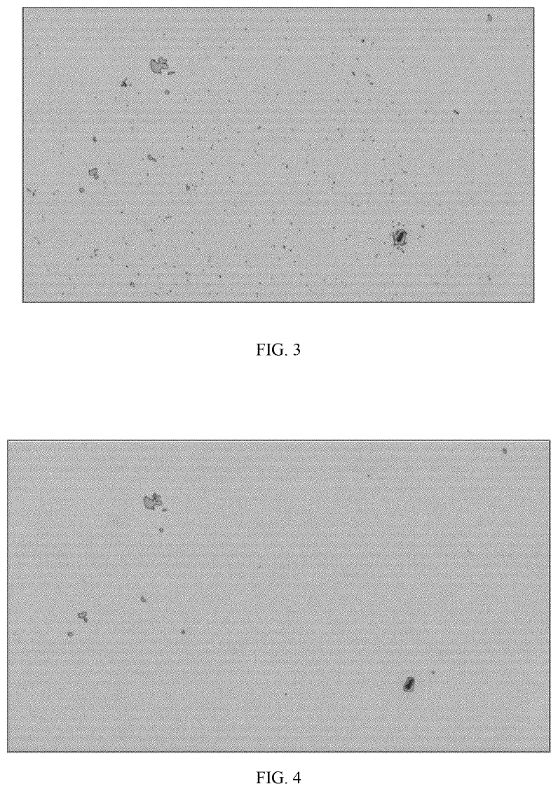

12. Define the thresholding parameter with the current set parameters. Generally, 0-145 is a good starting point; however, this will depend on several factors, including light intensity, exposure time, dryness of the membrane, and any other factors affecting the background light intensity. You should adjust the segmentation until you capture the entirety of all particles seen while not segmenting regions of the background where no particles are visibly distinguishable. See FIGS. 2-4 for example images of good and poor segmentation.

Thresholding parameters were determined based on a combination of blank membranes and real samples that were produced using the pilot manufacturing process.

13. The minimum and maximum equivalent circular diameter can now be defined for analysis. To increase computational speed the size bins considered should be restricted to 10 μm-42000 μm.

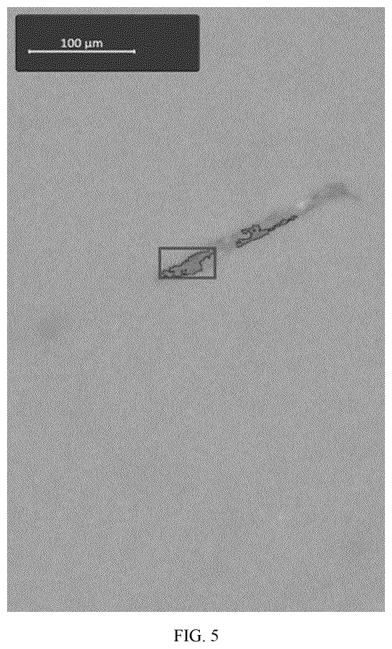

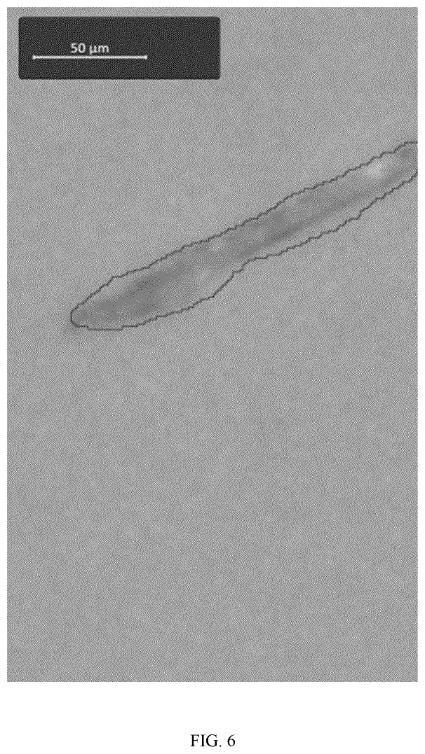

14. Perform a manual inspection of the particles with the particle editing menu for proper segmentation of all particulates >100 μm. The red overlay area indicating the segmentation region should overlap the particle fully and should not allow nearby particles to be combined into a larger one. Manually separate these in the inspection step. Also look for any large particles that were split into multiple smaller regions because of variable brightness or combined with background regions of the membrane due to the particle having a kind of shadow effect which gets segmented with the real particle. Representative images are shown as FIG. 5 and FIG. 6.

Take special care to remove any optical artifacts that may be captured based on the settings that can be obviously identified as not particles. If a more specialized analysis for particle classification is required, then the thresholding parameters would need to be adjusted.

15. Total particulate counts will be displayed by bins in terms of equivalent circular diameter.

16. Save the CZI file associated with the run.

17. Save the PDF associated with the sample which will record sample information, largest particle gallery for largest fiber and largest particle, a table of the particle counts by equivalent circular diameter, the full stitched membrane image and membrane occupancy rate, and the critical collection parameters used during image acquisition.

18. If the results are deemed satisfactory, retain the membrane in the 50 mm petrislide container that was prepared until the sample is deemed suitable for release by the functional area manager.

19. Repeat collection procedures as needed for subsequent samples.

### Microscope Shutdown Procedure

Ensure that all current runs are saved, and samples have been removed from the microscope stage.

If the microscope is needed for further use that day, the brightness can be reduced to zero and the light source will enter standby mode.

If the microscope is no longer needed for further analysis that day, turn off all power modules for the microscope.

Shut down the Zen Core software package.

### Results and Reporting

### Assay Acceptance Criteria

Particulate counts for suspension samples should meet the following criteria as specified by USP <788> and USP <790>:
Particles with ECD>10 μm: <3000 particles
Particles with ECD>25 μm: <300 particles
Particles with ECD>100 μm: 0 particles

Report the number of particles per vial and per method blank for >10 μm, >25 μm, and >100 μm in the appropriate electronic lab notebook (ELN) entry. The ELN should include the PDF report generated by the software which captures the results, collection parameters, and images of any particles >25 μm.

### Example 2

Reagents should be prepared for each sample required in addition to enough reagent to run three destructive treatment blanks and method blanks at the same replicate number as for suspension samples. Destructive treatment blanks require the use of the same volume of destructive reagent buffers without the use of any suspension sample or carrier liquid. Method blanks require the same volume of destructive reagent buffers applied to a sample of carrier liquid of equivalent volume to the suspension sample.

### A) Preparation of Dissolution Media (e.g., 0.5% v/v Polysorbate 20 in 1× Phosphate Buffered Saline (PBS) pH 7.4)

Dissolution media will be specific to each product and described in the associated dissolution protocol and batch release protocol.

Volumes should be scaled according to the number of samples to be run in a single day; this preparation is appropriate for a single sample. The reagent should be prepared fresh on the day of analysis.

1. Add 2.5 mL of Polysorbate 20 (PS20) directly into 500 mL PBS bottle using a positive displacement pipette.

2. Mix the PBS container at 60 RPM on the nutating mixer for 10 minutes.

3. Store mixture at room temperature until required in Section C).

B) Preparation of Protein Destructive Reagent (e.g., 4 M Urea, 10 mM tris(2-carboxyethyl) phosphine hydrochloride (TCEP))

The reagent should be prepared fresh on the day of analysis to avoid the generation of particles from containers, as well as the degradation of urea which will oxidize.

Volumes should be scaled according to the number of samples to be run in a single day; this preparation is appropriate for five (5) samples.

1. Add 24.02 g urea to a 50 mL conical tube using an analytical balance.

2. Fill the 50 mL conical tube to the 50 mL mark with class 1 ultrapure water.

3. Cap the conical tube and shake horizontally using a nutating mixer at 30 rpm for 60 minutes or until solution is fully dissolved.

Freshly prepared urea solutions undergo endothermic dissolution; allow at least 30 minutes for the urea solution to equilibrate to room temperature before use.

4. Add 286.66 mg TCEP to a separate 50 mL conical tube using an analytical balance.

5. Fill the 50 mL conical tube to the 50 mL mark with ultrapure water.

6. Cap the conical tube and shake horizontally using a nutating mixer at 60 rpm for 30 minutes.

7. Combine the urea and TCEP solutions in a 1 L 0.22 μm PES disposable filter flask in the Biosafety Level 2 Cabinet (BSC). First, vacuum filtering the urea solution followed by vacuum filtering the TCEP solution. Immediately close with the cap provided with the disposable vacuum filter.

8. Store at room temperature until use.

### C) Suspension Sample Dissolution and Membrane Filtration

Sections C) and D) should be performed for destructive treatment blanks and method blanks as well, using the same total volume of destructive reagents for both samples and blanks.

1. Vortex suspension for at least 60 seconds to homogenize sample within vial.

2. De-crimp vial and using a 250 μL positive displacement pipette, transfer 250 μL four times to 225 mL conical tube.

3. Add 3 mL of dissolution media into vial and close vial with the rubber stopper.

4. Vortex vial for 15 seconds at high speed. Transfer contents of vial to the same 225 mL conical tube from Step 2 using the 250 μL positive displacement pipette.

5. Dilute the solution to ˜20 mg/mL with dissolution media inside a BSC.

6. Mix the solution for 60 minutes at 60 rpm using a nutating mixer.

7. Dilute the solution to ˜10 mg/mL using 10% sodium dodecyl sulfate (SDS) inside a BSC. For example: a single 1 milliliter sample, 25 mL of dissolution media is combined with 25 mL of 10% SDS (5% SDS final concentration).

8. Mix the solution for 90 minutes at 60 rpm using a nutating mixer.

9. Transfer membrane filtration apparatus components to a BSC.

10. Filtration assembly components should be washed again within the BSC using the filterjet with water being pressure pumped through the filter.

11. Assemble the filtration apparatus as per FIG. 1. The filter for this analysis is a 0.45 μm poly vinylidene fluoride (PVDF).

12. Assemble the filtration apparatus as per FIG. 1 and add a single 0.45 μm PVDF filter membrane to the filtration apparatus (#3 in FIG. 1).

13. Turn on the vacuum and connect the tubing to the filtering cup connector.

14. Pour the entire contents of the dissolved suspension in the 225 mL Falcon tube directly into the filtration apparatus.

15. Wash the tube using the filterjet to remove any particles that may be stuck to the inside of the tube.

Use the pressure pumping setup to wash vials from the bottom of the vial in a side to side motion moving toward the rim of the vial.

16. Turn off vacuum and break the vacuum either using a diverting valve or by removing the tubing to release vacuum pressure.

### D) Destructive Treatment for Proteins

1. After the sample has been filtered and the negative pressure has equilibrated to atmospheric pressure, add 20 mL of protein destructive reagent (4 M urea, 10 mM TCEP) to the filtration apparatus containing the dissolved protein microparticle solution, cover and incubate at room temperature for 20 minutes.

2. Apply vacuum to pull the protein destructive reagent through the filter, then add 20 mL of ultrapure water onto the membrane while continuing to apply the vacuum to ensure complete removal of residual protein destructive reagent.

3. After the water has completely passed through the filter, add 20 mL of filtered reagent alcohol onto the membrane. Completely pull the filtered reagent alcohol through to ensure complete removal of residual ethyl oleate.

4. Apply vacuum for at least 30 seconds.

5. Remove membrane from filtration apparatus using clean tweezers and place into “PetriSlide for contamination analysis” (part number: PD1504700) with the lid slightly ajar in the BSC and allow to dry for at least 15 minutes or until dry.

6. After drying, the membrane is moved to the microscope stage and the PetriSlide lid is removed. Collection of micrographs can then proceed as described in the next section. If sample collection cannot occur after membrane drying, the cover of the PetriSlide can be fully closed and stored for future analysis.

### E) Instrument Startup for Zeiss Microscope

See Section D) of Example 1.

### Example 3

In this Example, a mixture of protein denaturation combined with enzyme digestion is utilized to reduce proteins (e.g., large proteins) into peptides. The suspension is first dissolved in an aqueous buffer such as PBS at concentrations ranging from 1-100 mg/mL, and this can be done in the presence of surfactants to further aid dissolution. The dissolution is performed on a gentle mixer or rocker such as a nutating or axial mixer for a time ranging anywhere from 30 seconds to 5 hours. This dissolved sample may contain a mixture of the protein monomers, protein aggregates, inherent protein sub-visible particles, process contaminants, etc. The dissolved sample is then treated with urea at concentrations ranging from 2-8M in the presence of heat to cause protein unfolding and help with solubility. The sample is then equilibrated to room temperature and a reducing agent is added to break the disulfide bonds in the sample. Reducing agents such as IAM and TCEP are used in dilute concentrations ranging from 5-100 mM. The sample is then digested by Lys-C, which is an endoprotease that cleaves proteins at the C-terminal of lysine residues. Following the digestion, the urea concentration is decreased to less than 1.6M to allow for effective trypsin digestion. The sample is then treated with trypsin for 3 hours and then sampled onto the filter membrane for imaging and downstream analytical measurements.

### Example 4

In this Example, trypsin is utilized to digest proteins into smaller peptides. The suspension sample is first dissolved in an aqueous buffer suited for the protein which is generally facilitated by axial mixing, The sample may then be reduced and treated with chaotropic agents followed by the enzyme, or the enzyme can be added directly post-dissolution of the suspension. This enzyme may be a commonly used enzyme such as trypsin, but additional enzymes and endoproteases may also be added depending on the protein conformation. The treatment of the enzyme may either be done in a static environment or a dynamic mixing environment and heat may be further added to facilitate this process. After digestion is complete, the sample may also be further treated with detergents such as sodium dodecyl sulfate to aid in the solubility and stability of the peptides in the aqueous solution. This sample may then be passed through a filter to ensure only contaminants of interest remain, and the protein is filtered out.

Depending on the protein structure of interest for treatment purposes, sample preparation and “destruction” may vary and either of the procedures highlighted to be non-destructive to extrinsic and intrinsic process contaminants may be used for treatment. Additionally, a combination of these treatments may be performed to further optimize the treatment and may be either done in sequence of one another or performed in a combination of one another as well. An example may be that some proteins may need an initial chaotropic treatment by protein denaturants such as urea or guanidine hydrochloride, followed by the enzyme digestion and detergent treatment prior to the filtration of the sample on the membrane.

Detergents and soaps (surfactants) play a role in their ability to interact with the oil fraction and emulsify the oil to create an oil-in-water emulsion. The presence of oil may complicate filtration and optimal sampling of the sample onto the membrane and these oil-in-water emulsions are able to flow through the filter pore size more efficiently without leaving significant oil residue. This further achieves optimal imaging and minimal carryover on the membrane enabling the imaging and analysis of the complete membrane without further manipulations. The ability of soaps and detergents to interact with oil and protein fraction of the sample, without destroying or affecting the sampling of the extrinsic and intrinsic contaminants of interest, contributes to the treatment success.

### Example 5

Example methods of protein degradation (e.g., hydrolysis, denaturation, fragmentation, etc.) are outlined in Table 3.

TABLE 3

| | | | Method 3 - | | Method 5 - |
|---|---|---|---|---|---|
| | Method 1 - | Method 2 - | SDS + Urea + | Method 4 - Urea, | Trypsin |
| Steps | SDS | SDS + Urea | Heat | protease approach | approach |
| 1 | Dissolve at | Dissolve at | Dissolve at | Dissolve at 100 mg/mL for | Dissolve at |
| | 20 mg/mL for | 20 mg/mL for | 20 mg/mL for | 1 hr | 20 mg/mL for |
| | 1 hr | 1 hr | 1 hr | | 1 hr |
| 2 | Dilute to | Add Urea & | Add Urea & | Add Urea | Trypsin |
| | 10 mg/mL with | SDS | SDS | | digestion for |
| | 10% SDS and | | | | 1.5 h |
| | mix 90 mins | | | | |
| 3 | On membrane | Mix 90 minutes | Heat sample at 70° | Heat samples at 70° | Wash steps |
| | urea treatment | and sample | C. for 30 mins | C. for 30 mins | |
| 4 | Wash steps | Wash steps | Wash steps | Equilibrate to room temp | |
| 5 | | | | Add 15 mM IAM | |
| 6 | | | | Lys-C (Lysyl Endopeptidase) | |
| | | | | digestion for 1 h | |
| 7 | | | | Reduce concentration | |
| | | | | of Urea to 1.6M | |
| 8 | | | | Trypsin digestion for 3 h | |
| 9 | | | | Wash steps | |

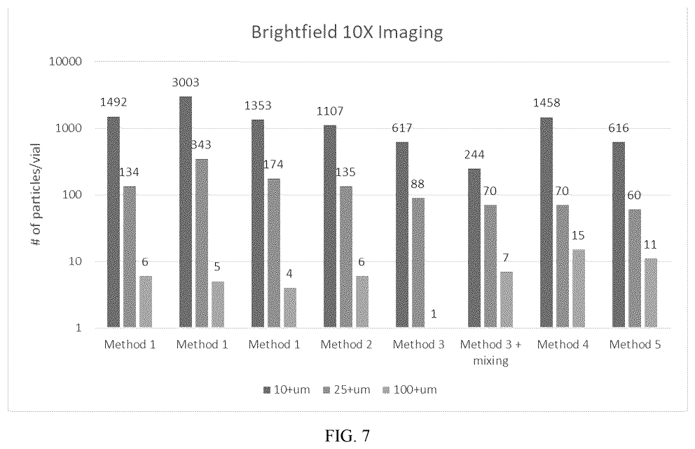

FIG. 7 shows the number of particles of various sizes per vial identified by using a brightfield microscope with a 10× objective lens after degrading the protein, based on the methods outlined in Table 3.

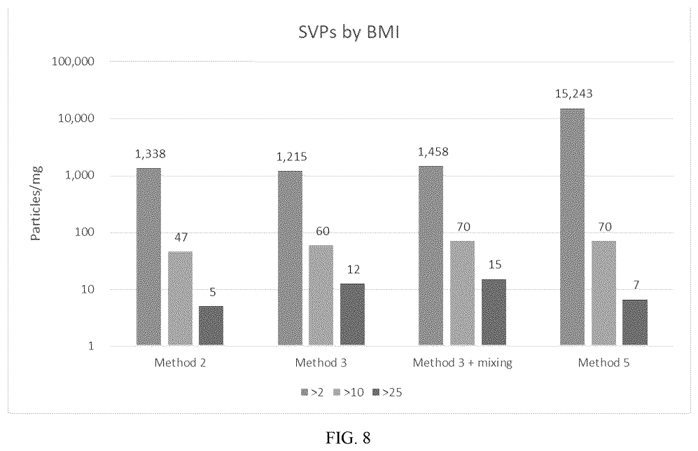

FIG. 8 shows the number of subvisible particles per mg of various sizes (i.e., >2 μm, >10 μm, >25 μm) identified by using Background Membrane Imaging (BMI) after degrading the protein, based on the methods outlined in Table 3.

### Materials and Methods

### Equipment

| | | | CAT/Part |
|---|---|---|---|
| Amount | Item/Description | Vendor | Number (PN) |
| 1 | Automated Microscope Equipped with the following: | Zeiss | AxioImager |
| | 10x objective | | M2M |
| | AxioCam Color 705 | | |
| | AxioCam Pol 705 | | |
| | LED light source configured in the episcopic configuration. | | |
| | Polarization cube | | |
| | Bright field cube | | |
| | Nomarski differential interference contrast cube | | |
| 1 | Positive Displacement Pipette. Microman E Positive | Gilson | FD10005 |
| | Displacement Pipette | | |
| 1 | Analytical balance. XSR series | Mettler | XSR105DU |
| | | Toledo | |
| 1 | Nutating Mixer | Fisherbrand | 88-861-043 (or |
| | | | equivalent) |
| 1 | Orbital shaker | Labnique | MT-201-BX |
| 1 | Filtration Apparatus Synthware ™ includes: | Fisher | F10401L |
| | Clamp | Scientific | |
| | Sintered glass filtering cup | | |
| | Filtering cup-300 mL | | |
| | Conical flask-1000 mL | | |
| 1 | Class II biosafety cabinet. 6' Purifier Logic + class II | Labconco | 303680121 |
| | type B2 biosafety cabinet with base stand | | |
| 1 | Straight Flat Tip Forceps. Designed for handling | Fisherbrand | 16-100-112 |
| | labels or SMD components | | |
| 1 | Pressure vessel accessories. Includes: | EMD | XX67000PK |
| | pressure relief valve | Millipore | |
| | pressure gauge | | |
| | hex nipple | | |
| | ball valve | | |
| | quick-release nipple | | |
| | hose adapter | | |
| 1 | Filterjet dispenser. Accessories for filter holders for | EMD | XX6702500 |
| | sample preparation | Millipore | |
| 1 | ¼ in. NPTM to M Ler locking nickel/chrome plated. | EMD | XX3002570 |
| | Accessories for filter holders for sample preparation | Millipore | |
| 1 | Masterflex Easyload Peristaltic pump | Masterflex | 77200-60 |

### Materials—Reusable and Consumable

| | | | CAT/Part |
|---|---|---|---|
| Amount | Item/Description | Vendor | Number |
| 1 | 0.2 μm pore size Whatman Polycap AS 36 | Cytiva | 6705-3602 (or |
| | Capsule Filter | | equivalent) |
| 1 | 0.8 μm NPTM to M Ler Lock Syringe Filter for | MilliporeSigma | XX3002570 |
| | aqueous filtration | | |
| 1 | 1000 mL Stericup Quick Release-GP Sterile | Millipore | S2GPU11RE |
| | Vacuum Filtration System. 0.22 μm pore size, | | |
| | polyethersulfone membrane, radio-sterilized | | |
| 4 | 125 mL Nalgene HDPE PP Lid | Thermo | 156- |
| | | Scientific | 125W/N/LP |
| 1 pack | 47 mm 0.65 um PVDF membrane filter | Millipore | DVPP04700 |
| 1 | Compressed nitrogen or house supplied at NLT | Approved | N/A |
| | 40 PSI | nitrogen gas | |
| | | supplier | |
| 1 pack | Petrislide Container | Millipore | PD1504700 |
| 1 pack | 0.22 um MILLIPAK GAMMA GOLD | Millipore | MPGL04GK2 |
| 1 | 0.2 um Millex PTFE filter | Millipore | SLFG025NB |
| 2 | 20 mL (24 mL) HENKE-JECT Luer lock syringe | HENKE SASS | 4200-X00V0 |
| | | WOLF | |
| 2 | 5 mL (6 mL) HENKE-JECT Luer Lock syringe | HENKE SASS | 4050-X00V0 |
| | | WILF | |

### Raw Materials, Solutions and Chemicals

| | | | | CAT/Part |
|---|---|---|---|---|
| Amount | | Item/Description | Vendor | Number |
| 480 | grams | Urea 99.3%. Denaturant. | Thermo Fisher | 036429.36 |
| | | | Scientific | |
| ~1 | L | Phosphate Buffered Saline. Gibco ™ | Thermo Fisher | 10010-031 |
| | | | Scientific | |
| 5 | mL | Polysorbate 20. Surfactant. | Emprove | 8.17072.1000 |
| | | | Essential | (or equivalent) |
| 2.5 | grams | Tris (2-carboxyethyl) phosphine | Thermo Fisher | 363830100 (or |
| | | hydrochloride. Reducing agent. | Scientific | equivalent) |
| 1 | L | 20% Sodium Dodecyl Sulfate | Thermo Fisher | 151-21-3 |
| | | | Scientific | |
| ~1 | L | WFI Quality Water | Cytiva | SH31191.03 |

### Example 6

### Demonstration of the Feasibility of Sieving Membrane Microscopy for Visible Particulate Detection

### Summary

The feasibility of employing a sieving membrane microscopy method to monitor visible particles per the requirements of USP <790> was tested. In this experiment, a blinded test set of singly-spiked vials and blanks was prepared by one analyst and the method was carried out by a second analyst. The second analyst correctly identified 86% of the spiked vials, demonstrating that a sieving membrane microscopy can identify visible particles in carrier liquid (e.g., non-aqueous liquid).

### INTRODUCTION

The current destructive membrane microscopy method has a ˜70-80% success rate of identifying visible particles. The method involves multiple contact surfaces where visible particles could be lost. In this experiment, a membrane microscopy method that uses a nylon sieve as the membrane to be imaged was tested. The larger pore size of the nylon sieve, 60 μm as compared to 0.65 μm, enables the microparticle suspension product to be filtered directly onto the sieve without dissolution or destructive steps, therefore reducing the number of contact surfaces. A drawback of this method is that subvisible particles cannot be monitored, as they will flow through the sieve.

### Methods

### Filter IPA (Isopropyl Alcohol)

1. In a fume hood, filter IPA using the peristaltic pump and the 0.22 μm MILLIPAK® Gamma Gold filter. Make sure to prewet the filter. Run the peristaltic pump at 300 mL/min.

### Prepare Blinded Sample Set

2. Obtain 9 glass vials. In 3 of the glass vials use forceps to place a single 500 μm blue lab coat fiber. In an additional 3 vials, use forceps to place a single rubber standard particle. Leave the remaining 3 vials empty. Visually inspect the vials to make sure they have the particles that they are supposed to.

3. Create an Excel file and list out the nine samples. Assign each sample a random 4 digit number. Sort samples in numerical order. Register new entities in Benchling as non-standard samples. For the rubber standards, make sure to include the initial vial number in the description of the entity.

4. Using a 20 ml syringe and PTFE syringe filter, filter carrier liquid into a 25 mL centrifuge tube. Make sure to discard the first 4 mL of carrier liquid.

5. Use a 1 mL positive displacement pipette to fill the vials with 2 mL of carrier liquid.

6. Cap and crimp the vials. Label with the assigned label.

### Membrane Filtration

7. Set up the filtration apparatus with the steel mesh grid support rather than the glass frit.

8. Attach the filtration apparatus to the vacuum regulator. Keep the valve between the vacuum regulator and the filtration apparatus closed.

9. Use forceps to transfer a 60 μm nylon sieve onto the filtration support.

10. Prime the filter by pouring 50 mL of filtered IPA onto the nylon sieve. Check the vacuum pressure on the vacuum regulator. If the vacuum is not on, open the valve between the house vacuum line and the regulator. Open the valve such that the vacuum pressure on the regulator is approximately 3 Hg.

11. Pull vacuum by slowly opening the valve between the regulator and the filtration apparatus. When pulling vacuum, slowly open the valve to allow fluid in the filter cup to flow through the nylon sieve at a steady pace. These instructions apply to all vacuum steps in this method.

12. Stop the vacuum by closing the valve.

13. Vortex the sample vial for at least 10 seconds and then decrimp the vial. Transfer sample from the vial to the nylon sieve with a 250 μL positive displacement pipette. Do not dispose of the tip yet. Do not pull vacuum.

14. Add 3 mL of filtered IPA to the sample vial using a 1 mL pipette. Cap the vial with the rubber stopper. Vortex sample vial for at least 10 seconds.

15. Use the 250 μL positive displacement pipette and the saved tip to transfer 1 mL of IPA from the sample vial to filter cup.

16. Transfer the remaining 2 mL of IPA from the sample vial to the filter cup using the 1 mL pipette.

17. Pull the vacuum. Close the valve.

18. Wash the nylon sieve with 50 mL of filtered IPA. Pull vacuum. Close the valve. Repeat one time.

19. Wash the nylon sieve with 50 mL of WFI (water for injection) water. Pull vacuum. Close the valve. Repeat one time.

20. Keep the vacuum on and remove the upper filtration cup.

21. Turn the vacuum off and carefully transfer the nylon sieve into the sample holder with forceps. Hold the sieve as flat as possible during the transfer.

22. Visually inspect the sieve and record if any particles are visible.

23. Allow the sieve to dry in the holder uncovered in the LFH (Laminar Flow Hood). Once the sieve is dry, place cover on holder.

### Image the Sieve

24. Set the Keyence microscope to 50× magnification.

25. Place the uncovered holder with the sieve on the stage of the Keyence microscope. Bring the sample into focus. Move the stage such that the center of the sieve is in the field of view. There will be a crosshair from the holder that makes this easy to visualize.

26. Select serial recording from the side menu. In “Step 1: Set XY range” select “Center of the FOV”. Check the box to specify the number of images. Select the manual option and select 12×12.

27. Under “Step 2: Start Stitching” select “Normal under “Image Format”. Click the “Start stitching” button with the green circle.

28. When the stitched image is finished, select the “Rec” button with the camera icon at the bottom of the screen to save the data.

### Results

TABLE 4
Visible Particle Detection: Each sample is listed with the identity
in the description column. Analyst 2 recorded if particles were
visible on the sieve by eye and during microscopic imaging.
If a particle was visible, the particle type was noted.

| | | Particle | Particle Visible |
|---|---|---|---|
| | | Visible on | on Sieve by |
| Sample | Description | Sieve by Eye? | Microscopy? |
| NST_Other3656 | FIber2 | fiber | fiber |
| NST_Other3657 | Rubber2 | Rubber and | rubber |
| | | another VP | |
| NST_Other3658 | Blank1 | none | none |
| NST_Other3659 | Fiber1 | none | none |
| NST_Other3660 | Rubber3 | rubber | rubber |
| NST_Other3661 | Blank3 | none | none |
| NST_Other3662 | Fiber3 | fiber | fiber |
| NST_Other3663 | Rubber1 | rubber | rubber |
| NST_Other3664 | Blank2 | none | none |

### Conclusions

All rubber particles and two of three fiber contaminants were found, demonstrating that this is a viable method for sampling VPs (visible particles) in carrier liquid and possibly suspension. The washing step in this method can be optimized to ensure as much carrier liquid as possible is removed from the sieve to make imaging easier, especially when performing automated particle identification. Additionally, the method in this Example may be practiced using ethanol instead of isopropyl alcohol.

### Example 7

### Demonstration of Destructive Membrane Microscopy for Visible Particulate Detection

### Summary

Testing of visible particles (>250 μm) is important for maintaining product compliance and ensuring compliance with USP<790>. Rubber and fiber particles were intentionally spiked into 2R vials and filled with carrier liquid to determine the detection rate of two distinct types of visible particles which have different morphological properties. The counts obtained from the counting of the micrographs were then evaluated against the known spiked amounts to demonstrate an overall detection rate of ˜78% for the two particle types evaluated.

In some embodiments, once a sample has been subjected to the method disclosed in this Example, the sample could not be used by a subject such as a patient.

### Methods

Testing fibers with a full destructive membrane microscopy (DMM) process.
Add five fibers individually into a single vial.
Repeat the previous step three times, generating four total vials.
2 mL of filtered carrier liquid was added to each vial
Analyst 1 tests the first two vials and analyst 2 tests the two remaining vials.

Test of rubber particles with full DMM process
A section of VITON® tubing was cut up (ranging from 300-600 μm) using scissors and added into vials. The four vials used in this study each contained five particles.
2 mL of filtered carrier liquid was added to each vial
Analyst 1 tests the first two vials and analyst 2 tests the two remaining vials.

### Results

TABLE 5
Results from two analysts performing DMM analysis
for vials spiked with >250 μm fibers.

| | | | | | Average |
|---|---|---|---|---|---|
| | A1 | A2 | | | Detection |
| | detected | detected | true | Detection | rate |
| Sample | instances | instances | instances | rate % | (fibers) |
| 1 | 3 | N/A | 5 | 60% | 0.7 |
| 2 | 4 | N/A | 5 | 80% | |
| 3 | N/A | 5 | 5 | 100% | 0.8 |
| 4 | N/A | 3 | 5 | 60% | |
| Overall | N/A | N/A | N/A | 75% | 0.75 |
| detection | | | | | |
| rate | | | | | |

TABLE 6
Results from two analysts performing DMM analysis
for vials spiked with >250 μm rubbers.

| | | | | | Average |
|---|---|---|---|---|---|
| | A1 | A2 | | | Detection |
| | detected | detected | true | Detection | rate |
| Sample | instances | instances | instances | rate % | (rubbers) |
| 1 | 4 | N/A | 5 | 80% | 0.8 |
| 2 | 4 | N/A | 5 | 80% | |
| 3 | N/A | 3 | 5 | 60% | 0.8 |
| 4 | N/A | 5 | 5 | 100% | |
| Overall | N/A | N/A | N/A | 80% | 0.8 |
| detection | | | | | |
| rate | | | | | |

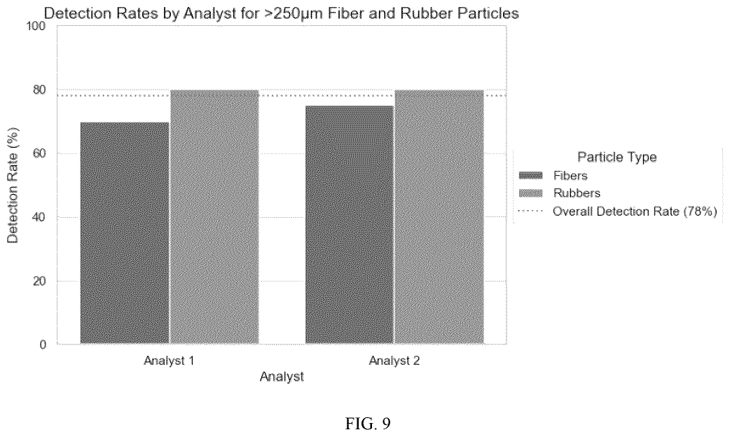

FIG. 9 shows detection rates of >250 μm particles.

### Conclusion

The results of this study demonstrate that the detection rate for visible particles (>250 μm) does not vary depending on the morphological properties of the particles. Elongated fibers exhibited an overall detection rate of 75%. Meanwhile more granular contaminants, in this case rubber particles, were detected at an 80% rate. These findings highlight the effectiveness of the DMM process in identifying various contaminants at comparable rates, despite distinct physical properties that could have otherwise proven to be a challenge. The methodology used aligns with USP compliance requirements, reinforcing the reliability of the approach in evaluating particulate contamination.

### REFERENCES

1) USP <787> Subvisible Particulate Matter in Therapeutic Protein Injections USPNF 2022 Iss. 1
2) USP <788> Particulate Matter in Injections USP41-NF36
3) USP <790> Visible Particulates in Injections USP41-NF36
4) USP <1787> Subvisible Particulate Matter in Therapeutic Protein Injections USPNF 2021 Iss. 1
5) USP <1788> Methods for the Determination of Subvisible Particulate Matter USPNF 2021 Iss. 1
6) USP <1788.2> Membrane Microscope Method for the Determination of Subvisible Particulate Matter USPNF 2021 Iss. 1
7) USP <1790> Visual Inspection of Injections USPNF 2022 Iss. 1
8) PDA Technical Report No. 79 Particulate Matter Control in Difficult to Inspect Parenteral March 2018

The teachings of all patents, published applications and references cited herein are incorporated by reference in their entirety.

While example embodiments have been particularly shown and described, it will be understood by those skilled in the art that various changes in form and details may be made therein without departing from the scope of the embodiments.

1. A method of detecting particulate contaminants in particles or a composition comprising particles, wherein the particles comprise a therapeutic or diagnostic agent, and wherein the particulate contaminants do not comprise the therapeutic or diagnostic agent, the method comprising:
a) mixing the particles or the composition comprising particles with a dissolution media under conditions in which the particles dissolve to produce a mixture comprising soluble therapeutic or diagnostic agent;
b) adding at least one destructive reagent to the mixture comprising the soluble therapeutic or diagnostic agent to degrade the therapeutic or diagnostic agent;
c) filtering the mixture through a filtration apparatus comprising a first membrane filter; and
d) inspecting the surface of the first membrane filter for the presence of particulate contaminants.

2. The method of claim 1, wherein the method comprises detecting particulate contaminants in a composition comprising particles, wherein the composition comprises particles suspended in a liquid.

3. The method of claim 1, wherein the therapeutic or diagnostic agent is a protein.

4. The method of claim 1, wherein the therapeutic or diagnostic agent is an antibody or a fragment thereof.

5. The method of claim 1, wherein the particulate contaminants are greater than about 2 μm in average diameter.

6. (canceled)

7. The method of claim 1, wherein the dissolution media comprises a surfactant.

8.-10. (canceled)

11. The method of claim 1, wherein the dissolution media is 0.5% v/v polysorbate 20 in 1× phosphate buffered saline (PBS) at a pH of about 7.4.

12. The method of claim 1, wherein mixing the particles or the composition with a dissolution media comprises diluting the particles or the composition to a therapeutic or diagnostic agent concentration of from about 1 mg/ml to about 50 mg/ml using the dissolution media.

13.-14. (canceled)

15. The method of claim 1, wherein the dissolution media is mixed with the particles or the composition using a nutating mixer or an orbital mixer.

16. (canceled)

17. The method of claim 1, wherein the dissolution media is mixed with the particles or the composition for about 90 minutes.

18. The method of claim 1, wherein the adding comprises diluting the mixture comprising the soluble therapeutic or diagnostic agent to a therapeutic or diagnostic agent concentration of from about 1 mg/ml to about 50 mg/ml using a first destructive reagent.

19.-20. (canceled)

21. The method of claim 18, wherein the first destructive reagent comprises sodium dodecyl sulfate, urea, or a combination thereof.

22. The method of claim 18, wherein the concentration of the first destructive reagent is from about 2 M to about 8 M.

23.-27. (canceled)

28. The method of claim 1, wherein the first membrane filter has a pore size of from about 0.1 μm to about 1 μm.

29.-32. (canceled)

33. The method of claim 18, wherein the adding further comprises contacting a second destructive reagent with the soluble therapeutic or diagnostic agent.

34.-46. (canceled)

47. The method of claim 1, wherein the method further comprises adding at least one enzyme to the mixture comprising the soluble therapeutic or diagnostic agent and the at least one destructive reagent to digest the therapeutic or diagnostic agent.

48.-57. (canceled)

58. A method of detecting particulate contaminants in particles or a composition comprising particles, wherein the particles comprise a therapeutic or diagnostic agent, and wherein the particulate contaminants do not comprise the therapeutic or diagnostic agent, the method comprising:
a) mixing the particles or the composition comprising particles with a dissolution media under conditions in which the particles dissolve to produce a mixture comprising soluble therapeutic or diagnostic agent;
b) adding at least one enzyme to the mixture comprising the soluble therapeutic or diagnostic agent to digest the therapeutic or diagnostic agent;
c) filtering the mixture through a filtration apparatus comprising a first membrane filter; and
d) inspecting the surface of the first membrane filter for the presence of particulate contaminants.

59.-74. (canceled)

75. A method of dissolving particles comprising a therapeutic or diagnostic agent, comprising:
a) adding a dissolution media to particles or a composition comprising particles suspended in a liquid;
b) mixing the dissolution media with the particles or the composition, thereby dissolving the particles to produce a solution comprising soluble therapeutic or diagnostic agent,
wherein the dissolution media comprises non-ionic surfactant.

76.-84. (canceled)

85. A method of preparing a destructive reagent, comprising
a) mixing a denaturant with a liquid to produce a first solution comprising a denaturant, wherein the denaturant comprises urea;
b) mixing a reductant with a liquid to produce a second solution comprising the reductant, wherein the reductant comprises tris(2-carboxyethyl) phosphine hydrochloride, beta-mercaptoethanol, dithiotheriol, or a combination thereof;
c) filtering the first and second solutions using a filtration apparatus, thereby obtaining a destructive reagent.

86.-99. (canceled)

100. A method of preparing a destructive reagent, comprising
a) mixing a first denaturant with a second denaturant to produce a first solution;
b) adding a reductant to the first solution to produce a second solution comprising the reductant, wherein the reductant comprises tris(2-carboxyethyl) phosphine hydrochloride, beta-mercaptoethanol, dithiotheriol, or a combination thereof;
c) filtering the second solution using a filtration apparatus, thereby obtaining a destructive reagent.

101.-107. (canceled)
